SEQUENCE LISTING (175 sequences)

SEQ ID NO: 1 - protein, 206 aa, synthetic construct
EGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 2 - protein, 155 aa, synthetic construct
TKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 3 - protein, 155 aa, synthetic construct
TKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 4 - protein, 208 aa, synthetic construct
STINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 5 - protein, 113 aa, synthetic construct
PIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 6 - protein, 156 aa, synthetic construct
NQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 7 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 8 - protein, 156 aa, synthetic construct
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 9 - protein, 176 aa, synthetic construct
FTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 10 - protein, 156 aa, synthetic construct
NQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 11 - protein, 200 aa, synthetic construct
GHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 12 - protein, 236 aa, synthetic construct
YEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 13 - protein, 137 aa, synthetic construct
GMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 14 - protein, 153 aa, synthetic construct
KYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 15 - protein, 163 aa, synthetic construct
AVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 174 aa, synthetic construct
GANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 17 - protein, 207 aa, synthetic construct
TDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 18 - protein, 127 aa, synthetic construct
ITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 19 - protein, 234 aa, synthetic construct
GSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 20 - protein, 161 aa, synthetic construct
SGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 21 - protein, 156 aa, synthetic construct
GDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 22 - protein, 156 aa, synthetic construct
ILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 23 - protein, 155 aa, synthetic construct
TKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 24 - protein, 208 aa, synthetic construct
DDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 25 - protein, 113 aa, synthetic construct
PIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 26 - protein, 113 aa, synthetic construct
PIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 27 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 28 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 29 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 30 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 31 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 32 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 33 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 34 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 35 - protein, 156 aa, synthetic construct
TKKVGIVDTTFARVDMASAAILTLKMESPNIKIIRKTVPGIKDLPVACKKLLEEEGCDIVMALGMPGKAKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 215 aa, synthetic construct
SDTDINNQLKARLKVIPVIAIDNAEDIIPLGKVLAENGLPAAEITFRSSAAVKAIMLLRSAQPEMLIGAGTILNGVQALAAKEAGATDFVVSPGFNPNTVRACQIIGIDIVPGVNNPSTVEAQALEMGLTTLKFFPAEASGGISMVKSLVGPYGDIRLMPTGGITPSDNIDNYLAIPQVLACGGTWMVDKKLVTRNGEWDEIARLTREIVEQVNP

SEQ ID NO: 37 - protein, 119 aa, synthetic construct
PIFTLNTNIKATDDVPSDFLSLTSRLVGLILSKEPGSYVAVHINTDQQLSFGGSTNPAAFGTLMSIGGIEPSDKNREDHSAVLFDHLNAMLGIPKNRMYIHFVNDLNDGDDVGWNGTTF

SEQ ID NO: 38 - protein, 166 aa, synthetic construct
NQHSHKDYHETVRIAVVRARWHADIVDACVEAFEIAMAAIGGDRFAVDVFDVPGAYEIPLHARTLAETGRYGAVLGTAFVVNDGGIYRDHEFVASAVIDGMMNVQLSDTGVPVLSAVLTPHREYREDSADEHDHREFFAAHFAVKGVEAARACIEILANAREKIAA

SEQ ID NO: 39 - protein, 219 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTDILKLFPGEVVGPQEFVKEAMKGPFPNVKFVPTGGVNDLDNDVCEKWFKDAGVLAVGVGSKDALVKEGTDKPDEVREKDAKAEKFVEKKEIRGCTE

SEQ ID NO: 40 - protein, 170 aa, synthetic construct
NQHSHKDYHETVRIAVVRARWHAEIVDACVSAFEAAMARDIGGDRFAVDVFDVPGAYEIPLHARTLAETGRYGAVLGTAFVVNDGGIYRDHEFVASAVIDNGMMNVQLSDNTGVPVLSAVLTPHRENYRDEDKSDKAHDTLLFLALFAVKGMEAARACVEILAAREKIAA

SEQ ID NO: 41 - protein, 158 aa, synthetic construct
GEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 42 - protein, 183 aa, synthetic construct
SQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 43 - protein, 102 aa, synthetic construct
EEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 44 - protein, 121 aa, synthetic construct
VRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 45 - protein, 176 aa, synthetic construct
SKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 46 - protein, 121 aa, synthetic construct
VRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 47 - protein, 171 aa, synthetic construct
RITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 48 - protein, 122 aa, synthetic construct
PHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 49 - protein, 157 aa, synthetic construct
ESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 50 - protein, 112 aa, synthetic construct
PVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 51 - protein, 102 aa, synthetic construct
EEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 52 - skipped

SEQ ID NO: 53 - protein, 202 aa, synthetic construct
EELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 54 - protein, 251 aa, synthetic construct
VAADIGAGLADALTAPLDHKDKGLKSLTLEDSISQNGTLTLSAQSAERTFKAGDKDNSLNTGKLKNDKISRFDFIRQIEVDGQLITLESGEFQVYKQSHSALTALQTEQVQDSEHSGKMVAKRQFRIGDIAGEHTSFDKLPKDVMATYRGTAFGSDDAGGKLTYTIDFAAKQGYGKIEHLKSPELNVDLAAANIEQDEKHHAVISGSVLYNQDEKGSYSLGIFGEKFQEVAGSAEVKTANGIHHIGLAAKQ

SEQ ID NO: 55 - protein, 248 aa, synthetic construct
VAADIGTGLADALTAPLDHKDKGLQSLTLDQSVRKNEKLKLAAQGAEKTYGNGDSLNTGKLKNDKISRFDFIRQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKRHAVISGSVLYNQNEKGSYSLGIFGGQAQEVAGSAEVETANGIHHIGLAAKQ

SEQ ID NO: 56 - protein, 248 aa, synthetic construct
VAADIGTGLADALTAPLDYKDKGLQSLMLDQSVRKNEKLKLSAQGAEKTYGNGDSLNTGKLKNDKVSRFDFIRQIEVDGQLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVDLATVDIKPDEKHHAVISGSVLYNQDEKGSYSLGIFGGKAQEVAGSAEVKTANGIRHIGLAAKQ

SEQ ID NO: 57 - protein, 248 aa, synthetic construct
VAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 58 - protein, 247 aa, synthetic construct
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSFDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 59 - protein, 248 aa, synthetic construct
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 60 - protein, 250 aa, synthetic construct
VAADIGTGLADALTTPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKTDSLINQRSFLVSGLGGEHTAFNQLPGGKAEYHGKAFSSDDPNGRLHYTIDFTNKQGYGRIEHLKTPEQNVELASAELKADEKSHAVILGDTRYGSEEKGTYHLALFGDRAQEIAGSATVKIGEKVHEIGIAGKQ

SEQ ID NO: 61 - protein, 247 aa, synthetic construct
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 62 - protein, 250 aa, synthetic construct
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKIDSLINQRSFLVSGLGGEHTAFNQLPVGKAEYHGKAFSSDDAGGKLTYTIDFAAKQGHGKIEHLKSPELNVELATAYIKPDEKHHAVISGSVLYNQDEKGSYSLGIFGGQAQEVAGSAEVETANGIRHIGLAAKQ

SEQ ID NO: 63 - protein, 249 aa, synthetic construct
VAADIGAGLADALTAPLDHKDKSLQSLTLDQSVRKNEKLKLAAQGAEKTYGNGDSLNTGKLKNDKVSRFDFIHQIEVDGQLITLESGEFQVYKQSHSALTALQTEQEQDPEHSGKMVAKRRFKIGDIAGEHTSFDKLPKDVMATYRGTAFGSDDAGGKLTYTIDFAAKQGHGKIEHLKSPELNVELATAYIKPDEKHHAVISGSVLYNQDEKGSYSPRYLWRKSPRSCRAARKWKPQTAYTISALAAKQ

SEQ ID NO: 64 - protein, 248 aa, synthetic construct
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 65 - protein, 247 aa, synthetic construct
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDAGGKLTYTIDFAAKQGHGRIEHLKTPEQNVELASAELKADEKSHAVILGDTRYGGEEKGTYHLALFGDRAQEIAGSATVKIREKVHEIGIAGKQ

SEQ ID NO: 66 - protein, 247 aa, synthetic construct
VAADIGAGLADALTAPLDHKDKGLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 67 - protein, 248 aa, synthetic construct
VTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 68 - protein, 248 aa, synthetic construct
VAADIGAGLADALTAPFDHKDKGLQSLTLDQSVRKNEKLKLAAQGAEEIYGNGDSLDTGKLKNDKVSRFDFIRQIEVDGQLITLESGEFQVYKQSHSALTALQTEQVQDSEHSRKMVAKRQFRIGDIAGEHTSFDKLPEGGRATYRGTALGSDDAGGKLIYTIDFAAKQGHGKIEHLKSPELNVDLAAAYIKPDEKHHAVISGSVLYNQAEKGSYSLGIFGGKAQEVAGSAEVKTVNGIRHIGLAAKQ

SEQ ID NO: 69 - protein, 248 aa, synthetic construct
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKTPEQNVELASAELKADEKSHAVILGDTRYGGEEKGTYHLALFGDRAQEIAGSATVKIREKVHEIGIAGKQ

SEQ ID NO: 70 - protein, 247 aa, synthetic construct
VAAEIGAGLDDALTAPLDHKDKSLRSLTLDQSILKNEKLKLAAHGAENPYGNGDRLYTGKLKNDKVSRFDFIRPIEVDGKVITLESGEFQIYKQDHSAVVALQIEKISNPDKIDGLINQRSFLVSGLGGEHTAFNQLPSGKAEYHGKAFSSDHAGGKLTYTIDFAAKQGHGKIEHLKTPEQNVELASAELKADEKHHAVILGDTRYGGEEKGTYHLALFGDRAQEIAGRATVDKGKGSRQIGIAGKQ

SEQ ID NO: 71 - protein, 248 aa, synthetic construct
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQEQDLEHSRKMVAKRRFKIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVKTANGIHHIGLAAKQ

SEQ ID NO: 72 - protein, 248 aa, synthetic construct
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQDHSALTALQIEKIQNSDHSGKMIAKRSFIVGGLGGEHTSFDQLPEGGREYHRGTAFRSNDAEKRLTYTIYFATKDGNGKIDHLHSPELSVLAAATYIKTDSQRHAFCASSVIYAKTKERTYSLFFRSAPTKEVARRPEIKSIKCINYLGLANKK

SEQ ID NO: 73 - protein, 248 aa, synthetic construct
VAADIGAGLADALTAPLDHKDKGLQSLTLNQSVRKKEKLKLAAQGAEKTYGNGDSLSTGKLKNDKVSRFDFIRQIEVDGQLITLESGEFQVYKQSHSALTALQTEQVQDSEHSGKMVAKRRFRIGDIAGEHTSFDKLPEGGRATYRGTAFSSDDAGGKLIYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 74 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 75 - protein, 247 aa, synthetic construct
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 76 - protein, 250 aa, synthetic construct
VAADIGTGLADALTTPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 77 - protein, 250 aa, synthetic construct
VAADIGAGLADALTAPLDHKDKGLKSLTLEDSIPQNGTLTLSAQGAEKTFKAGGKDNSLNTGKLKNDKISRFDFVQKIEVDGQTITLASGEFQIYKQDHSAVVALQIEKINNPDKTDSLINQRSFLVSGLGGEHTAFNQLPGGKAEYHGKAFSSDDPNGRLHYTIDFTNKQGYGRIEHLKTPEQNVELASAELKADEKSHAVILGDTRYGSEEKGTYHLALFGDRAQEIAGSATVKIGEKVHEIGIAGKQ

SEQ ID NO: 78 - protein, 250 aa, synthetic construct
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 79 - protein, 248 aa, synthetic construct
VAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 80 - protein, 248 aa, synthetic construct; identical to SEQ ID NO: 64

SEQ ID NO: 81 - protein, 247 aa, synthetic construct
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 82 - protein, 247 aa, synthetic construct
VAADIGAGLADALTTPLDHKDKSLQSLTLDQSVRKNEKLKLAAQGAEKTYGNGDSLNTGKLKNDKVSRFDFIRQIEVDGQTITLASGEFQIYKQNHSAVVALQIEKINNPDKIDSLINQRSFLVSGLGGEHTAFNQLPDGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 83 - protein, 250 aa, synthetic construct
VAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 84 - protein, 247 aa, synthetic construct
VAADIGTGLADALTAPLDHKDKGLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 85 - protein, 251 aa, synthetic construct; identical to SEQ ID NO: 74

SEQ ID NO: 86 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 87 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFDKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 88 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFDKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVKTVNGIRHIGLAAKQ

SEQ ID NO: 89 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFDKLPKDVMATYRGTAFGSDDAGGKLTYTIDFAAKQGHGKIEHLKSPELNVDLAAAYIKPDEKHHAVISGSVLYNQDEKGSYSLGIFGEKAQEVAGSAEVKTANGIHHIGLAAKQ

SEQ ID NO: 90 - protein, 251 aa, synthetic construct; identical to SEQ ID NO: 74

SEQ ID NO: 91 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKRHAVISGSVLYNQAEKGSYSLGIFGGKAQEVAGSAEVKTVNGIRHIGLAAKQ

SEQ ID NO: 92 - protein, 251 aa, synthetic construct
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVELATAYIKPDEKHHAVISGSVLYNQDEKGSYSLGIFGGQAQEVAGSAEVETANGIHHIGLAAKQ

SEQ ID NO: 93 - protein, 248 aa, synthetic construct
VAADIGAGLADALTAPLDHKDKGLQSLTLDQSVRKNEKLKLAAQGAEKTYGNGDSLNTGKLKNDKISRFDFIRQIEVDGQLITLESGEFQVYKQSHSALTALQTEQVQDSEDSGKMVAKRQFRIGDIAGEHTSFDKLPEDVRATYRGTAFGSDDAGGKLIYTIDFAAKQGHGKIEHLKSPELNVDLAAADIKPDEKRHAVISGSVLYNQDEKGSYSLGIFGGKAQEVAGSAEVETANGIQHIGLAAKQ

SEQ ID NO: 94 - protein, 249 aa, synthetic construct
AADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIREKVHEIGIAGKQ

SEQ ID NO: 95 - protein, 250 aa, synthetic construct
VAADIGTGLADALTTPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 96 - protein, 250 aa, synthetic construct
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSGLGGEHTAFNQLPGGKAEYHGKAFSSDDPNGRLHYSIDFTKKQGYGRIEHLKTPEQNVELASAELKADEKSHAVILGDTRYGSEEKGTYHLALFGDRAQEIAGSATVKIGEKVHEIGIAGKQ

SEQ ID NO: 97 - protein, 250 aa, synthetic construct; identical to SEQ ID NO: 96

SEQ ID NO: 98 - protein, 248 aa, synthetic construct
VAADIGAGLADALTAPLDHKDKGLQSLTLDQSVRKNEKLKLAAQGAEETYGNGDSLNTGKLKNDKVSRFDFIRQIEVDGQLITLENGEFQVYKQSHSALTALQTEQVQDSEHSGKMVAKRQFRIGDIAGEHTSFDKLPEGGRATYRGTAFGSDDTGGKLTYTIDFAAKQGHGKIEHLKSPELNVELATAYIKPDEKRHAVISGSVLYNQDEKGSYSLGIFGGQAQEVAGSAEVETANGIQHIGLAAKQ

SEQ ID NO: 99 - protein, 266 aa, synthetic construct
GTIKAGVETSRSVTHYGAQANRVKTATEIADLGSKIGFKGQEDLGNGLKAIWQLEQKAYVSGTDTGWGNRQSFIGLKGGFGKVRVGRLNNILKDTGGFNPWEGKSYYLGLSNIAQPEERPISVRYDSPEFAGFSGSVQYVPNDNSSKNHSESYHAGFNYKNSGFFVQYAGSYKRHSYTTEKHQVHRLVGGYDHDALYASVAVQQQDAKLAWPDDNSHNSQTEVAATAAYRFGNVTPRVSYAHGFKGSVYDADNDNTYDQVVVGAEY

SEQ ID NO: 100 - protein, 266 aa, synthetic construct
GAIKAGVETSRSVAYHGAQADRVKTTTEIADLGSKIGFKGQEDLGNGLKAIWQLEQKAYVSGTKEGWGSRQSFIGLKGGFGKVRVGHLNNILKDTDGFNPWEGKSYYLGLSNIAQPEERHVSVRYDSPEFAGFSGSVQYVPNDNSGKNRSESYHAGFNYKNSGFFVQYAGFYKRHNYTTEKHQVHRLVGGYDHDALYASVAVQQQDAKLTWSNDNSHNSQTEVAATAAYRFGNVTPRVSYAHGFKGSVYDADNDNTYDQVVVGAEY

SEQ ID NO: 101 - protein, 254 aa, synthetic construct
GTIKAGVETSRSVAYHGAQADRVKTTTEIADLGSKIGFKGQEDLGNGLKAIWQLEQKAYVSGTKEGWGSRQSFIGLKGGFGKVRVGHLNNILKDTDGFNPWEGKSYYLGLSNIAQPEERHVSVRYDSPEFAGFSGSVQYVPNDNSGKNRSESYHAGFNYKNSGFFVQYAGSYKRHNYTTEKHQVHRLVGGYDHDALYASVAVQQQDAKLTWSNDNSHNSQTEVAATAAYRFGNVTPRVSYAHGFKGSVYDADND

SEQ ID NO: 102 - protein, 171 aa, synthetic construct
WGNRQSFIGLKGGFGKVRVGRLNSVLKDTDGFNPWEGKSYYLGLSNIAQPEERHVSVRYDSPEFAGFSGSVQYVPNDNSGKNHSESYHAGFNYKNSGFFVQYAGFYKRHNYTTEKHQVHRLVGGYDHDALYASVAVQQQDAKLTWRNDNSHNSQTEVATTVAYRFGNVTPR

SEQ ID NO: 103 - protein, 266 aa, synthetic construct
GTIKAGVETSRSVAHHGAQADRVKTATEIADLGSKIGFKGQEDLGNGLKAIWQLEQKAYVSGTDTGWGNRQSFIGLKGGFGKVRVGRLNNILKDTDGFNPWEGKNYYLGLSNIAQPEERHVSVRYDSPEFAGFSGSVQYVPNDNSGKNHSESYHAGFNYKNSGFFVQYAGSYKRHNFTTEKHQVHRLVGGYDHDALYASVAVQQQDAKLAWSDDNSHNSQTEVAATAAYRFGNVTPRVSYAHGFKGSVYDADNDNTYDQVVVGAEY

SEQ ID NO: 104 - protein, 492 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 105 - protein, 496 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 106 - protein, 500 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 107 - protein, 508 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGSGGSGGSGSGGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 108 - protein, 484 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 109 - protein, 488 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 110 - protein, 492 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 111 - protein, 500 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGSGGSGSGGSGGSGSGGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 112 - protein, 271 aa, synthetic construct
MGSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQGGLEHHHHHH

SEQ ID NO: 113 - protein, 496 aa, synthetic construct; identical to SEQ ID NO: 105

SEQ ID NO: 114 - protein, 223 aa, synthetic construct
EKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 115 - protein, 813 aa, synthetic construct
ATGGGATCTTCCGGTGGTGGCGGTTCTGGCGGCGGTGGCGTTACGGCGGACATTGGTACTGGTCTCGCGGACGCCCTCACCGCACCTCTCGACCACAAAGACAAAGGTCTGAAATCTCTGACGCTGGAAGACTCTATCTCTCAGAACGGTACCCTCACGCTGTCTGCTCAGGGTGCGGAAAAAACCTACGGTAACGGTGACTCTCTGAACACCGGTAAACTGAAAAACGACAAAGTTTCTCGTTTTGACTTCATCCGTCAGATCGAAGTAGACGGTCAGCTCATCACCCTCGAATCTGGTGAATTCCAGGTTTACAAACAGTCTCACTCTGCGCTGACGGCCCTGCAGACCGAACAAGAACAGGACCCGGAACACTCCGAAAAAATGGTTGCGAAACGTCGTTTCCGTATCGGTGATATCGCTGGCGAACACACCTCTTTTGACAAACTGCCGAAAGACGTTATGGCGACCTACCGTGGTACCGCGTTTGGTAGCGATGACGCGGGTGGTAAGCTGACGTACACCATCGACTTCGCGGCTAAACAGGGTCACGGTAAAATCGAGCACCTCAAATCTCCGGAACTGAATGTTGACCTGGCCGTAGCTTACATCAAACCGGATGAGAAACATCACGCGGTTATCTCTGGCTCCGTGCTGTACAACCAGGACGAAAAAGGTTCTTACAGCCTGGGTATCTTCGGTGAAAAAGCTCAGGAAGTGGCAGGTAGCGCGGAAGTTGAAACCGCGAATGGTATCCACCACATCGGCCTGGCAGCCAAGCAAGGCGGCCTCGAGCACCACCACCACCACCAC

SEQ ID NO: 116 - protein, 1488 aa, synthetic construct
ATGGGATCTTCCGGTGGTGGCGGTTCTGGCGGCGGTGGCGTTACGGCGGACATTGGTACTGGTCTCGCGGACGCCCTCACCGCACCTCTCGACCACAAAGACAAAGGTCTGAAATCTCTGACGCTGGAAGACTCTATCTCTCAGAACGGTACCCTCACGCTGTCTGCTCAGGGTGCGGAAAAAACCTACGGTAACGGTGACTCTCTGAACACCGGTAAACTGAAAAACGACAAAGTTTCTCGTTTTGACTTCATCCGTCAGATCGAAGTAGACGGTCAGCTCATCACCCTCGAATCTGGTGAATTCCAGGTTTACAAACAGTCTCACTCTGCGCTGACGGCCCTGCAGACCGAACAAGAACAGGACCCGGAACACTCCGAAAAAATGGTTGCGAAACGTCGTTTCCGTATCGGTGATATCGCTGGCGAACACACCTCTTTTGACAAACTGCCGAAAGACGTTATGGCGACCTACCGTGGTACCGCGTTTGGTAGCGATGACGCGGGTGGTAAGCTGACGTACACCATCGACTTCGCGGCTAAACAGGGTCACGGTAAAATCGAGCACCTCAAATCTCCGGAACTGAATGTTGACCTGGCCGTAGCTTACATCAAACCGGATGAGAAACATCACGCGGTTATCTCTGGCTCCGTGCTGTACAACCAGGACGAAAAAGGTTCTTACAGCCTGGGTATCTTCGGTGAAAAAGCTCAGGAAGTGGCAGGTAGCGCGGAAGTTGAAACCGCGAATGGTATCCACCACATCGGCCTGGCAGCCAAGCAAGGCGGCTCTGGTGGTTCTGGCGGCAGCGGCGGTTCCGAGAAAGCAGCGAAAGCTGAAGAAGCTGCACGTAAAATGGAAGAACTGTTCAAAAAACACAAAATCGTTGCAGTTCTGCGTGCGAACTCTGTTGAAGAAGCGATCGAAAAAGCGGTTGCGGTTTTCGCGGGTGGTGTCCACCTCATCGAAATCACCTTCACCGTGCCGGACGCAGATACCGTTATCAAAGCGCTGTCTGTTCTGAAAGAAAAAGGTGCGATCATCGGTGCGGGTACGGTTACCTCTGTAGAACAGGCGCGTAAGGCGGTTGAATCTGGCGCGGAATTCATCGTTTCTCCGCACCTCGACGAGGAAATCTCTCAGTTTGCGAAAGAGAAAGGTGTTTTCTACATGCCGGGTGTTATGACCCCGACGGAACTGGTGAAAGCGATGAAGCTGGGTCACACCATCCTGAAACTGTTTCCGGGTGAAGTTGTTGGTCCGCAGTTCGTAAAAGCCATGAAAGGTCCGTTCCCTAATGTTAAATTCGTTCCGACCGGCGGTGTCAACCTGGACAACGTAGCGGAATGGTTCAAAGCAGGTGTTCTCGCGGTTGGTGTTGGCTCTGCGCTGGTTAAAGGTACCCCGGACGAAGTTCGTGAAAAGGCAAAAGCGTTCGTTGAAAAAATCCGTGGTGCGACCGAACTCGAGCACCACCACCACCACCAC

SEQ ID NO: 117-131 - skipped (15 entries)

SEQ ID NO: 132 - protein, 203 aa, synthetic construct
MEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 133 - protein, 153 aa, synthetic construct
KYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 134 - protein, 150 aa, synthetic construct
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 135 - protein, 119 aa, synthetic construct
NLAEKMYKAGNAMYRKGQYTIAIIAYTLALLKDPNNAEAWYNLGNAAYKKGEYDEAIEAYQKALELDPNNAEAWYNLGNAYYKQGDYDEAIEYYKKALRLDPRNVDAIENLIEAEEKQG

SEQ ID NO: 136 - protein, 119 aa, synthetic construct
EEAELAYLLGELAYKLGEYRIAIRAYRIALKRDPNNAEAWYNLGNAYYKQGDYREAIRYYLRALKLDPENAEAWYNLGNALYKQGKYDLAIIAYQAALEEDPNNAEAKQNLGNAKQKQG

SEQ ID NO: 137 - protein, 119 aa, synthetic construct
NSAEAMYKMGNAAYKQGDYILAIIAYLLALEKDPNNAEAWYNLGNAAYKQGDYDEAIEYYQKALELDPNNAEAWYNLGNAYYKQGDYDEAIEYYEKALELDPNNAEALKNLLEAIAEQD

SEQ ID NO: 138 - protein, 187 aa, synthetic construct
TDPLAVILYIAILKAEKSIARAKAAEALGKIGDERAVEPLIKALKDEDALVRAAAADALGQIGDERAVEPLIKALKDEEGLVRASAAIALGQIGDERAVQPLIKALTDERDLVRVAAAVALGRIGDEKAVRPLIIVLKDEEGEVREAAAIALGSIGGERVRAAMEKLAERGTGFARKVAVNYLETHK

SEQ ID NO: 139 - protein, 119 aa, synthetic construct
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYEKALELDPENLEALQNLLNAMDKQG

SEQ ID NO: 140 - protein, 279 aa, synthetic construct
IEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEKFMARAIMAIAILAAKAIEAIYRLADNHTSPTYIEKAIEAIEKIARKAIKAIEMLAKNITTEEYKEKAKKIIDIIRKLAKMAIKKLEDNRT

SEQ ID NO: 141 - protein, 153 aa, synthetic construct
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 142 - protein, 119 aa, synthetic construct
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 143 - protein, 6 aa, synthetic construct
HHHHHH

SEQ ID NO: 144 - protein, 203 aa, synthetic construct
MEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 145 - protein, 8 aa, synthetic construct
GGSGGSGS

SEQ ID NO: 146 - protein, 12 aa, synthetic construct
GGSGGSGGSGGS

SEQ ID NO: 147 - protein, 16 aa, synthetic construct
GGSGGSGSGGSGGSGS

SEQ ID NO: 148 - protein, 24 aa, synthetic construct
GGSGGSGSGGSGGSGSGGSGSGGS

SEQ ID NO: 149 - protein, 17 aa, synthetic construct
GGGGSGGGGSGGGGSGG

SEQ ID NO: 150 - protein, 15 aa, synthetic construct
GGGGGSGGGSGGGGS

SEQ ID NO: 151 - protein, 15 aa, synthetic construct
GGGGSGGGGSGGGGS

SEQ ID NO: 152 - protein, 7 aa, synthetic construct
GGSGEKP

SEQ ID NO: 153 - protein, 7 aa, synthetic construct
GGSGQKP

SEQ ID NO: 154 - protein, 10 aa, synthetic construct
GGSGGSGEKP

SEQ ID NO: 155 - protein, 10 aa, synthetic construct
GGSGGSGQKP

SEQ ID NO: 156 - skipped

SEQ ID NO: 157 - protein, 4 aa, synthetic construct
GSGS

SEQ ID NO: 158 - protein, 8 aa, synthetic construct
GGSGGSGS

SEQ ID NO: 159 - protein, 12 aa, synthetic construct
GSGGSGSGSGGS

SEQ ID NO: 160 - protein, 12 aa, synthetic construct
GGSGGSGGSGGS

SEQ ID NO: 161 - protein, 15 aa, synthetic construct
GGGGSGGGGSGGGGS

SEQ ID NO: 162 - protein, 15 aa, synthetic construct
GGGGGSGGGSGGGGS

SEQ ID NO: 163 - protein, 16 aa, synthetic construct
GGSGGSGSGGSGGSGS

SEQ ID NO: 164 - protein, 17 aa, synthetic construct
GGGGSGGGGSGGGGSGG

SEQ ID NO: 165 - protein, 24 aa, synthetic construct
GGSGGSGSGGSGGSGSGGSGSGGS

SEQ ID NO: 166 - protein, 7 aa, synthetic construct
GGSGEKP

SEQ ID NO: 167 - protein, 7 aa, synthetic construct
GGSGQKP

SEQ ID NO: 168 - protein, 10 aa, synthetic construct
GGSGGSGEKP

SEQ ID NO: 169 - protein, 10 aa, synthetic construct
GGSGGSGQKP

SEQ ID NO: 170 - protein, 30 aa, synthetic construct
KSDELLGSGGSGSGSGGSEKAAKAEEAARK

SEQ ID NO: 171 - protein, 25 aa, synthetic construct
MELLILKANAITTILTAVTFCFASG

SEQ ID NO: 172 - protein, 19 aa, synthetic construct
MSWKVMIIISLLITPQHGL

SEQ ID NO: 173 - protein, 17 aa, synthetic construct
MKAILVVLLYTFTTANA

SEQ ID NO: 174 - protein, 18 aa, synthetic construct
MPISILLIITTMIMASHC

SEQ ID NO: 175 - protein, 15 aa, synthetic construct
MFVFLVLLPLVSSQC

(19) United States
(12) Patent Application Publication
(10) Pub. No.: US 2025/0222092 A1
(43) Pub. Date: Jul. 10, 2025

(54) PROTEIN NANOSTRUCTURE VACCINE

(71) Applicant: Icosavax, Inc., Seattle, WA (US)

(72) Inventors: Julia McKechnie, Seattle, WA (US); Quinton Dowling, Seattle, WA (US); Daniel Ellis, Seattle, WA (US); Andrew Feldhaus, Seattle, WA (US); Ralph Braun, Seattle, WA (US); Douglas Holtzman, Seattle, WA (US)

(21) Appl. No.: 19/010,881

(22) Filed: Jan. 6, 2025

Related U.S. Application Data
(60) Provisional application No. 63/699,683, filed on Sep. 26, 2024.
(60) Provisional application No. 63/618,986, filed on Jan. 9, 2024.

(51) Int. Cl. A61K 39/095; A61P 37/04; B82Y 5/00; C07K 1/14; C07K 14/22

(52) U.S. Cl. CPC A61K 39/095; A61P 37/04; B82Y 5/00; C07K 1/14; C07K 14/22; C07K 2319/40

(57) ABSTRACT

Provided are protein nanostructures that display Neisseria meningitidis factor H binding protein (fHBP). The protein nanostructure may be a two-component icosahedral nanostructure. Further provided are vaccine compositions, methods of manufacturing, and methods of use, e.g., immunizing a subject to generate a protective immune response to Neisseria meningitidis.

### CROSS REFERENCE TO RELATED APPLICATIONS

This application claims priority to U.S. provisional patent application 63/618,986, filed on Jan. 9, 2024, and to U.S. provisional patent application 63/699,683, filed on Sep. 26, 2024, the contents of each of which are incorporated herein by reference in their entireties.

### SEQUENCE LISTING

The instant application contains a Sequence Listing which has been submitted electronically in XML format and is hereby incorporated by reference in its entirety. Said XML copy created on Dec. 19, 2024, is named 061291-521001WO.xml and is 166 KB in size.

### BACKGROUND

Neisseria meningitidis is a Gram-negative encapsulated bacterium that can cause sepsis, meningitis, and death. N. meningitidis can be classified into at least 12 serogroups (including serogroups A, B, C, 29E, H, I, K, L, W-135, X, Y and Z) based on chemically and antigenically distinctive polysaccharide capsules. Strains with five of the serogroups (A, B, C, Y, and W135) are responsible for the majority of disease.

Neisseria meningitidis is a major cause of endemic cases and epidemics of meningitis and devastating septicemia. The symptoms can include cough, fever, nasal congestion, and shortness of breath. Meningococcal meningitis is a devastating disease that can kill children and young adults within hours despite the availability of antibiotics.

Immunogenic antigens include fHBP, NadA and NHBA. Factor H binding protein (fHBP) is the main inhibitor of the alternative complement pathway. To achieve this, fHBP binds the human complement regulator fH, a protein which is very abundant in the human plasma that coats all human tissues and prevents the complement attack C3 to self-tissues. By coating N. meningitidis with fH, fHBP makes the bacterium invisible to C3 and allows it to grow undisturbed in the human blood and to cause sepsis, or to cross the blood-brain barrier and cause meningitis. fHBP protects meningococci from complement-mediated death in human serum experiments, but has also been shown to protect meningococci from antimicrobial peptides in vitro. fHBP is key to the pathogenesis of N. meningitidis.

Although effective vaccines directed against capsular polysaccharide exist for several scrogroups of pathogenic N. meningitidis, such approaches have failed to provide a universal solution for serogroup B (MenB) due to its capsular polysaccharide's similarity to a polysialic acid present on the surface of human cells. Studies of a current meningococcal group B vaccine (GlaxoSmithKline's Bexsero (4CMenB)) show the vaccine prevents disease among recipients (but only about 75%), but it does not affect pharyngeal carriage of the bacterium Neisseria meningitidis on a population level. Furthermore, there is no vaccine that is effective across all the main scrogroups.

Consequently, there is a need for improved immunogenic compositions against meningococcal scrogroups A, B, C, Y, W135 and X.

### SUMMARY

Provided are protein nanostructure display for a Neisseria meningitidis fHBP protein and related compositions and methods.

In an aspect, the disclosure provides a protein nanostructure, including a first component and optionally a second component, wherein the first component includes a factor H binding protein (fHBP), optionally of Neisseria meningitidis or Neisseria gonorrhoeae or any Neisseria bacterium, and a first polypeptide comprising a first assembly domain, and wherein the second component includes a second polypeptide including a second assembly domain, wherein the first assembly domain and the second assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 1.

In embodiments, the fHBP includes a sequence that is at least 70%, at least 80%, at least 90%, at least 95%, at least 96%, at least 97%, at least 98%, at least 99%, or 100% identical to any polypeptide sequence in Table 2.

In embodiments, the first component includes a fusion protein including, in N to C-terminal order, the fHBP, a polypeptide linker, and a I53-50A assembly domain.

In embodiments, the polypeptide linker includes between 6 and 18 amino acids.

In embodiments, the polypeptide linker includes about 8 amino acids.

In embodiments, the polypeptide linker includes about 12 amino acids.

In embodiments, the polypeptide linker includes about 16 amino acids.

In embodiments, the polypeptide linker includes 8 amino acids.

In embodiments, the polypeptide linker includes 12 amino acids.

In embodiments, the polypeptide linker includes 16 amino acids.

In embodiments, the polypeptide linker is a glycine-serine (Gly-Ser) linker.

In embodiments, the polypeptide linker is any sequence in Table 3.

In embodiments, the fusion protein includes a polypeptide sequence at least 85%, at least 90%, at least 95%, at least 96%, at least 97%, at least 98%, at least 99%, or 100% identical to any sequence in Table 4.

In embodiments, the first component is a trimeric component comprising three copies of the first polypeptide. In embodiments, the second component is a pentamer comprising five copies of the second polypeptide. In embodiments, the nanostructure comprises 20 copies of the first component. In embodiments, the nanostructure further comprises 12 copies of the second component.

In embodiments, the protein nanostructure is an icosahedral nanostructure.

In embodiments, the I53-50A assembly domain includes a polypeptide sequence at least 70%, at least 80%, at least 90%, at least 95%, at least 96%, at least 97%, at least 98%, at least 98%, at least 99%, or 100% identical to any one of SEQ ID NOs: 7, 29-31, 39, 53, 144, or 132.

In embodiments, the I53-50B assembly domain includes a polypeptide sequence at least 70%, at least 80%, at least 90%, at least 95%, at least 96%, at least 97%, at least 98%, at least 98%, at least 99%, or 100% identical to any one of SEQ ID NOs: 8, 32-34, or 40.

In an aspect, the disclosure provides a polynucleotide encoding the components of a protein nanostructure of the disclosure.

In embodiments, the polynucleotide is DNA.

In embodiments, the polynucleotide is RNA.

In embodiments, the polynucleotide is any sequence in Table 5.

In an aspect, the disclosure provides a pharmaceutical composition including a protein nanostructure or polynucleotide of the disclosure and one or more pharmaceutically acceptable diluents, adjuvants, or excipients.

In embodiments, the pharmaceutical composition is a vaccine.

In embodiments, the pharmaceutical composition includes an adjuvant.

In an aspect, the disclosure provides a method of immunizing a subject against infection by meningococcal disease, the method including administering a protein nanostructure, polynucleotide, or pharmaceutical composition of the disclosure.

In an aspect, the disclosure provides a fusion protein as used in a protein nanostructure of the disclosure or a polynucleotide encoding a fusion protein, or a host cell including such as polynucleotide.

In an aspect, the disclosure provides a method of manufacturing a vaccine, including culturing a host cell in a culture medium so that the host cell secretes the first component into the culture medial; and optionally purifying the first component from the culture media. In embodiments, the host cell is a bacterial cell. In embodiments, the bacterial cell is an E. coli cell.

In embodiments, the method includes mixing the first component with a second component, wherein the second component multimerizes with the first component to form a protein nanostructure; and optionally purifying the protein nanostructure.

In an aspect, the disclosure provides a kit including a pharmaceutical composition of the disclosure and instructions for use.

### BRIEF DESCRIPTION OF THE DRAWINGS

FIG. 1A shows an illustrative embodiment of a protein nanostructure according to the present disclosure.

FIG. 1B shows illustrative protein nanostructures.

FIG. 2 shows an SDS-PAGE gel of the soluble fHBP protein, CompA-fHBP, and fHBP VLPs. Each sample was run pre and post a freeze thaw cycle. Each polypeptide is of the expected size.

FIG. 3 shows the SYPRO signal over the temperature ramp from 15° C. to 95° C. for CompA-fHBP. The Tm was estimated at 57° C. and the Tonset at 52° C.

FIG. 4 shows the intensity distribution (DLS) of the fHBP VLPs. The VLPs were monodisperse and of the expected size.

FIG. 5 shows the nsEM image of fHBP VLPs. A subset of the VLPs were disrupted as a result of the processing of the samples during staining.

FIG. 6 shows graphs of the biolayer interferometry (BLI) with three antibodies, JAR4, JAR5, and JAR41, that bind fHBP.

FIG. 7 shows graphs of the hSBA titers in day 49 sera for a commercial MenB-FHbp Meningococcal Group B vaccine (Trumenba “MenB-FHbp”), soluble fHBP protein (“rfHbp”), CompA-fHBP, and fHBP VLPs against six MenB isolates. VLPs displaying fHBP antigen induced higher SBA titers than the other antigens tested for all isolates expressing variant 1 fHBPs.

FIG. 8 shows graphs of binding titers to peptide 55 and peptide 45 in day 49 sera for Trumenba (“MenB-FHbp”), soluble fHBP protein (“rfHbp”), CompA-fHBP, and fHBP VLPs. VLPs displaying fHBP antigen induced higher peptide 55 binding titers than the other antigens tested. Trumenba elicited higher peptide 45 binding titers than the other antigens tested.

FIG. 9 shows graphs evaluating the anti-peptide 55 IgG1 and IgG2a levels in mice immunized with rfHbp, fHbp-CompA, and fHbp VLPs. There was a general trend towards the fHbp VLP groups having higher IgG1 levels than the rfHbp and fHbp-CompA groups. IgG2a levels were significantly higher in mice immunized with fHbp VLPs compared to mice immunized with rfHbp or fHbp-CompA. Therefore, mice immunized with rfHbp and fHbp-CompA had a bias towards IgG1. In contrast, mice immunized with fHbp VLPs had relatively comparable levels of IgG1 and IgG2a.

### DETAILED DESCRIPTION

Provided are protein nanostructures that can be used as vaccines for Meningitis in which an fHBP protein is linked to, and thereby displayed on, a protein nanostructure, e.g., a designed protein nanostructure, e.g., a symmetric protein nanostructure. For example, the vaccine antigen may be a C-terminal fusion of the ectodomain of fHBP to a protein having an assembly domain for a one- or two-component de novo designed protein nanostructure, such as a two-component icosahedral protein nanostructure. Further provided are vaccine compositions, methods of manufacturing, and methods of use, e.g., immunizing a subject to generate a protective immune response to Neisseria meningitidis.

The protein nanostructures disclosed herein display potentially antigenic polypeptides intended to elicit immune responses to Neisseria meningitidis. In embodiments, the vaccines of the present disclosure are useful for preventing and/or decreasing the severity of infection by the bacterium.

### Definitions

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The practice of the technology will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. It is to be understood that this disclosure is not limited to the particular methodology, protocols, and reagents described, as these may vary, depending upon the context in which they are used by those of skill in the art.

All publications and patents mentioned herein are hereby incorporated by reference in their entirety as if each individual publication or patent was specifically and individually indicated to be incorporated by reference. In case of conflict, the present application, including any definitions herein, controls. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described. Specifically, features described in one section may be combined with features in any other section of the description.

While illustrative embodiments are described and depicted, it will be appreciated that various changes can be made to these illustrative embodiments without departing from the spirit and scope of the invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole.

The singular forms “a”, “an”, and “the” are intended to include the plural forms as well, unless the context clearly indicates otherwise.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10%, +/−5%, +/−3%, or +/−1% of the specified value.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

Throughout this specification, unless the context requires otherwise, the words “comprise”, “comprises” and “comprising” will be understood to imply the inclusion of a stated step or element or group of steps or elements but not the exclusion of any other step or element or group of steps or elements. For example, a composition “comprising” (or “which comprises”) components A, B, and C can consist of (i.e., contain only) components A, B, and C, or can contain not only components A, B, and C but also one or more other components.

As used herein, the term “consisting of” refers to including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The following description includes information that may be useful in understanding the present invention. It is not an admission that any of the information provided herein is prior art or relevant to the presently claimed inventions, or that any publication specifically or implicitly referenced is prior art.

The term “protein nanostructure,” as used herein, refers to symmetric protein assemblies in which the subunits self-assemble in aqueous solution, without requiring lipids or macromolecules other than the protein nanostructure for assembly. Illustratrative protein nanostructures are described in Hsia et al. Nature 35:136-9 (2016) and Bale et al. Science 353:389-394 (2016). In embodiments, the protein nanostructure is a one-component protein nanostructure, in which a single polypeptide type provides the building blocks to self-assemble to form the protein nanostructure. In embodiments, the protein nanostructure is a two-component protein nanostructure, in which two polypeptide types provide the building blocks to self-assemble to form the protein nanostructure. In embodiments, the polypeptide types include an assembly domain, which causes the polypeptide to form symmetric dimeric, trimeric, tetrameric, hetaxameric components, or another multimeric component. In a two-component nanostructure, the two components differ in the selection of an assembly domain. In embodiments, the assembly domain of the first polypeptide type causes the polypeptide to form a trimer; and the assembly domain of the second polypeptide type causes the polypeptide to form a pentamer.

In embodiments of, or relating to, one-component nanostructures, two or more copies of the component further symmetrically self-assembly to form the nanostructure. In embodiments of, or relating to, two-component nanostructures, two or more of each of the two, different components symmetrically self-assemble to form the nanostructure.

The term “protein nanostructure,” as used herein, refers to symmetric protein assemblies in which the subunits self-assemble in aqueous solution, without requiring lipids or macromolecules other than the protein nanostructure for assembly. Illustratrative protein nanostructures are described in Hsia et al. Nature 35:136-9 (2016) and Bale et al. Science 353:389-394 (2016). In embodiments, the protein nanostructure is a one-component protein nanostructure, in which a single polypeptide self-assembles to form the protein nanostructure. In embodiments, the protein nanostructure is a two-component protein nanostructure, in which two polypeptides self-assemble to form the protein nanostructure.

As used herein, the term “assembly domain” refers to the portion of a subunit of a component involved in forming the protein nanostructure through intra-component interations and interactions with either other copies of the same component (in a one-component nanostructure) or with other components (e.g., in a two-component nanostructure).

The term “icosahedral particle” refers to protein nanostructures having a core with icosahedral symmetry. I53 refers to an icosahedral particle constructed from pentamers and trimers. 152 refers to an icosahedral particle constructed from pentamers and dimers. T33 refers to a tetrahedral particle constructed from two sets of trimers. T32 refers to a tetrahedral particle constructed from trimers and dimers.

The potentially antigenic polypeptides may be attached to the core of the protein nanostructure either non-covalently or covalently, including as a fusion protein or by other means disclosed herein. Multimeric polypeptides may optionally be displayed along a symmetry axis of the protein nanostructure. Also provided are proteins and nucleic acid molecules encoding such proteins, formulations, and methods of usc.

The term “antigen” or “immunogen” refers to its plain and ordinary meaning of a compound or composition that induces an immune response, cellular or humoral, e.g., cytotoxic T lymphocyte (CTL) response, a B cell response (for example, production of antibodies that specifically bind the epitope), an NK cell response or any combinations thereof, when administered to or expressed in an immunocompetent subject. Antigens can include polypeptides (including glycoproteins). In aspects, an antigen is a polypeptide or polypeptide complex including at least one component designed to elicit an immune response. For example, an antigen can include one or more immunogenic epitopes associated with a bacterial pathogen. The term antigen, as used herein, is not limited to the portion of the polypeptide or polypeptide complex that contains antigenic epitopes. An “epitope” or “antigenic determinant” refers to its plain an ordinary meaning as the part of an antigen that is recognized by the immune system, specifically by antibodies, B cells, or T cells.

The terms “polypeptide,” “peptide” and “protein” are used interchangeably herein to refer to a polymer of amino acid residues and optionally one or more post-translational modifications (e.g., glycosylation) and/or other modifications (including but not limited to conjugation of the polypeptide moiety used as a marker-such as a fluorescent tag- or an adjuvant).

The term “isolated”, when applied to a polynucleotide or polypeptide, denotes that the polynucleotide or polypeptide is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high-performance liquid chromatography. A polynucleotide or polypeptide that is the predominant species present in a preparation is substantially purified.

The term “bacterium” is used according to its plain ordinary meaning within Microbiology and refers to a member of a group of unicellular microorganisms which have cell walls but lack organelles and an organized nucleus, including some that can cause disease (pathogenic). The term “bacterial infection” or “bacterial disease” refers to a disease or condition that is caused by a bacterium, such as bacterial meningitis caused by Neisseria meningitidis.

The term “variant” refers to a polypeptide having one or more insertions, deletions, or amino acid substitutions relative to a reference polypeptide, but retains one or more properties of the reference protein.

The term “antigenic variant” refers to a variant that has one or more epitopes in common with a reference polypeptide and/or generates the same or similar immune response when administered to a subject as a reference polypeptide.

The term “functional variant” refers to a variant that exhibits at least some of the activity as a reference polypeptide. For example, a functional variant of an assembly domain is able to promote multimerization and self-assembly to the same extent, or to similar extent, as a reference assembly domain and/or is able to multimerize and assembly with the same cognate assembly domains as a reference assembly domain.

As used herein, the term “bound” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary (a “linker”)). In embodiments, the two moieties are non-covalently bonded (e.g. through ionic bond(s), van der waal's bond(s)/interactions, hydrogen bond(s), polar bond(s), or combinations or mixtures thereof). For a fusion polypeptide that is N-terminally and C-terminally joined the linker is a peptide bond.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

Another method of covalently linking two polypeptides is the SpyTag/SpyCatcher system. The peptide SpyTag (13 amino acids) spontaneously reacts with the protein SpyCatcher (12.3 kDa) to form an intermolecular isopeptide bond between the pair. Polynucleotide sequence encoding either SpyTag or SpyCatcher can be recombinantly introduced into the polynucleotide sequence encoding polypeptides of interest, forming a fusion protein. These fusion proteins can be covalently linked when mixed in a reaction through the SpyTag/SpyCatcher system.

Using the Tag/Catcher pair, bioconjugation can be achieved between two recombinant proteins that would otherwise be restrictive or impossible with traditional direct genetic fusion between the two proteins. For example, issues regarding protein folding, suboptimal expression host, and specialized post-translational modifications can be alleviated by separating the production of the proteins with the modularity of the Tag/Catcher system.

The term “domain” refers to refers to any portion of a polypeptide that adopts a tertiary structure.

The terms “assembly domain” and “multimerize” refer to the ability of a polypeptide, or domain of a polypeptide, to form a tertiary structure with another polypeptide of domain of a polypeptide. In embodiments, assembly domains can form dimers, trimers, tetramers, pentamers, or hexamers and/or to form heteromers with other assembly domains.

The term “trimerization domain” refers to a assembly domain that forms trimers.

The term “assembly domain” refers to a assembly domain that, alone or with other assembly domains, forms a protein nanostructure.

The term “fragment” refers to a polypeptide having one or more N-terminal or C-terminal truncations compared to a reference polypeptide.

The term “functional fragment” refers to a fragment that retains at least one function of its reference polypeptide.

The term “amino acid substitution” refers to replacing a single amino acid in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94th residue, relative to a reference sequence, is arginine (Arg).

The terms “helix” or “helical” refer to an α-helical secondary structure in a polypeptide that is known to occur, or predicted to occur. For example, a sequence may be described as helical when computational modeling suggests the sequence is likely to adopt a helical conformation.

The term “component” refers to a protein, or protein complex, capable of assembly into a protein nanostructure under appropriate conditions (e.g., a fusion protein comprising an assembly domain).

The term “vaccine” refers to a composition that can provide active acquired immunity to and/or therapeutic effect (e.g. treatment) of a particular disease (e.g. bacterial meningitis) or a pathogen (e.g. Neisseria meningitides). A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating meningitis in a subject in need thereof). The administration of vaccines is referred to vaccination. In some examples, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus).

The term “pharmaceutically acceptable excipients” and “pharmaceutically acceptable carrier” refer to a substance that aids the administration of an active agent to and absorption by a subject and can be included in the compositions of the present disclosure without causing a significant adverse toxicological effect on the patient and can mean excipients approved by a regulatory agency of the Federal or a state government or listed in the U.S. Pharmacopeia or other generally recognized pharmacopeia for use in animals, and more particularly in humans. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “adjuvants” refers to a pharmaceutically acceptable substance that enhances the immune response to an antigen when co-administered with the antigen or administered before, during, or after administration of the antigen to a subject.

The term “TLR4 immunostimulant” refers to an adjuvant that stimulates Toll-like Receptor 4 (TLR4) in the immune cells of a subject to modulate an immune response e.g., Monophosphoryl Lipid A (MPL), Glucopyranosyl Lipid A (GLA), and/or synthetic lipid A (SLA). In embodiments, the antigen is a TLR4 immunostimulant.

The term “effective amount” refers to the amount of a composition that, when administered to a patient for treating a state, disorder or condition is sufficient to effect such treatment or when administered to a patient for generating an immune response is sufficient to generate such an immune response. The exact amounts will depend on the active ingredient, the state, disorder, or condition to be treated and its severity, and the age, weight, physical condition and responsiveness of the subject to be treated, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The terms “immunization” and “immunizing” refer to administering a composition to a subject in an amount sufficient to elicit, after one or more administering steps, a desired immune response. Immunization may comprise between one and ten, or more administrations (e.g., injections) of the composition, such as 1, 2, 3, 4, 5, 6, 7, 8, 9, 10, or more administrations. The first administration may elicit no detectable immune response as generally each subsequent administration will boost the immune response generated by prior administrations. The term “immunizing” as used herein includes post-exposure prophylaxis.

The term “protective immune response” refers to an immune response that prevents and/or reduces the severity of infection with a pathogen when the subject is later challenged with the pathogen, or to an immune response that generates a level of immune response that correlates with protection. For example, vaccination may generate a protective immune response if it results in production, in the plasma or serum, of the subject (e.g., human, pet, or agricultural animal), of neutralizing antibodies that protect the subject against subsequent infection and/or are present in a quantity observed to confer protection upon test subjects (e.g., New Zealand White (NZW) rabbits).

The term “polyclonal antibody response” refers to an antibody response comprising antibodies having more than one specificities and/or variation in their antibody sequences.

The term “neutralizing” (e.g., “neutralizing antibody response”) refers to antibodies that prevent infection and/or reduce the level of infection by a pathogen. A neutralizing antibody response can be measured either in in vitro assays (e.g., killing of target bacteria in the presence of the antibody) or in an in vivo assay (e.g., by determining a protective dose of an antibody through administering the antibody to a subject prior to challenge with an infective dose of a pathogen).

An antibody “binds to” or is “specific to” or “specifically binds” (used interchangeably herein) to a target (e.g., bacterial protein) are terms well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances.

The term “predetermined time” refers to an interval of time selected as appropriate for observing a particular effect. A predetermined time may be selected before, or during, an experiment or procedure.

The term “post-exposure prophylaxis” refers to administering an antigenic composition (e.g., a vaccine) to a subject previously exposed to and/or infected with a pathogen in order to elicit an immune response to protect against infection by the pathogen and/or decrease the severity of one or more symptoms of infection by the pathogen.

The term “administering” refers to providing a composition to a subject in a manner that permits the composition to have its intended effect. Administration for vaccination or post-exposure prophylaxis may be performed by intramuscular injection, intravenous injection, intraperitoneal injection, or any other suitable route.

“Co-administer” means that a composition described herein is administered at the same time, just prior to, or just after the administration of one or more additional therapies. The compositions provided herein can be administered alone or can be coadministered to the subject. Co-administration is meant to include simultaneous or sequential administration of the compounds individually or in combination (more than one compound). Thus, the preparations can also be combined, when desired, with other active substances (e.g., to reduce metabolic degradation).

The term “subject” refers to a human or non-human animal to which a composition may be administered for vaccination, treatment, or other purpose. In embodiments, the non-human animal is a non-human primate, rabbit, hamster, gerbil, pig, cow, sheep, goat, guinea pig, rat, mouse, squirrel, wolf, fox, horse, zebra, giraffe, elephant, cat, dog, llama, or ferret.

The term “manufacturing” refers to production of a recombinant polypeptide or protein nanostructure at any scale, including at least 25-mL, 50-mL, 1-L, 1,000-L, 50,000-L, or greater scale.

The terms “culturing” and “culture medium” refers to standard cell culture and recombinant protein expression techniques.

The term “host cell” refers to any cell capable of use in expression of a recombinant polypeptide or polynucleotide. In embodiments, the host cell is a bacterial cell. In embodiments, the bacterial cell is an E. coli cell.

The term “secretes” refers to the ability of host cells to release expressed polypeptides into the media in which they are cultured.

The term “signal peptide” refers to a polypeptide sequence, typically at the N terminus of a polypeptide expressed in a host cell, that directs the polypeptide to a particular cellular compartment. A signal peptide may be a secretion signal to cause the host cell to secrete the polypeptide into the media in which with host cell is cultured. The signal peptide can be the “native” signal peptide, a signal peptide that occurs in nature as part of the polypeptide. The signal peptide can be a sequence that does not naturally occur with the polypeptide as found in nature. Various signal peptides are known and it is within the skill of an ordinary artisan to select an appropriate signal peptide.

The term “purify” refers to separating a molecule from other substances present in a composition. Polypeptides may be purified by affinity (e.g., to an antibody or to a tag, e.g., using a His-tag capture resin), by charge (e.g., ion-exchange chromatography), by size (e.g., preparative ultracentrifugation, size exclusion chromatography), or otherwise.

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The terms “identity”, “identical”, and “sequence identity” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. For sequence comparison, typically one sequence acts as a reference sequence, to which test sequences are compared. The term “reference sequence” refers to a molecule to which a test sequence is compared.

Methods of sequence alignment for comparison and determination of percent sequence identity is well known in the art. Optimal alignment of sequences for comparison can be conducted, e.g., by the homology alignment algorithm of Needleman and Wunsch, (1970) J. Mol. Biol. 48:443.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

### EMBODIMENTS

Provided herein are protein nanostructures and protein nanostructure-based vaccines. In embodiments, the protein nanostructures of the present disclosure display an antigen capable of eliciting immune responses to N. meningococcus. In embodiments, vaccines of the present disclosure are useful for preventing or decreasing the severity of infection with Neisseria meningitidis. In particular, the antigens of the disclosure display the factor H Binding Protein (fHBP). The protein, variant thereof, or fragment thereof may be attached to the core of the protein nanostructure either non-covalently or covalently, including as a fusion protein or by other means disclosed herein. In embodiments, a linker connects the fHBP to a first polypeptide comprising a assembly domain. The linker may be any chemical linkage including but not limited to a polypeptide used to form a N-terminal or C-terminal fusion of the fHBP to the first polypeptide. The fHBP may optionally be displayed along a symmetry axis of the protein nanostructure. Also provided are proteins and nucleic acid molecules encoding such proteins, formulations, and methods of usc.

### Protein Nanostructures

The protein nanostructures of the present invention may comprise multimeric protein assemblies adapted for display of fHBP, or an antigenic fragment thereof. The protein nanostructures of the present invention comprise at least a first component and, optionally, a second component. The first component may include substitution of at least one amino acid residue or by addition at the N- or C-terminus of one or more amino acid residues. In embodiments the fusion protein comprises a protein sequence determined by computational methods. This first component may form the entire core of the protein nanostructure; or the core of the protein nanostructure may include a second component or third, fourth, fifth component and so on. In embodiments the first component is a trimeric component in which the assembly domains form trimers related by 3-fold rotational symmetry, and/pr the second component is a pentameric component, in which the assembly domains form pentamers related by 5-fold rotational symmetry. In embodiments, the combination of the two components form an “icosahedral particle” having I53 symmetry. Together these components may be arranged such that the members of each component are related to one another by symmetry operators. A general computational method for designing self-assembling protein materials, involving symmetrical docking of protein building blocks in a target symmetric architecture, is disclosed in Patent Pub. No. US 2015/0356240 A1.

The “core” of the protein nanostructure is used herein to describe the central portion of the protein nanostructure that links together the several copies of fHBP, or antigenic fragments thereof, displayed by the protein nanostructure. In an embodiment, the fusion protein comprises a first polypeptide comprising an fHBP or antigenic variant thereof, a linker, and a first assembly domain. In embodiments, the fHBP, or antigenic fragment thereof, is non-covalently or covalently linked to the assembly domain. For example, an antibody or antigenic fragment thereof may be fused to the first component and configured to bind a portion of the first component, or a chemical tag on the first component. For example, a streptavidin-biotin (or neuravidin-biotin) linker can be employed. Or various bioconjugate linkers may be used. In embodiments of the present disclosure, the antigen comprises further polypeptide sequences in addition to fHBP.

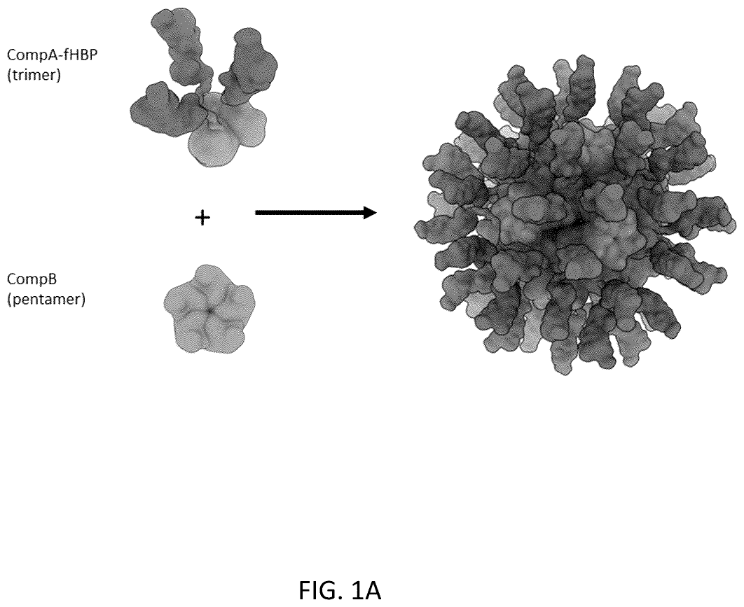

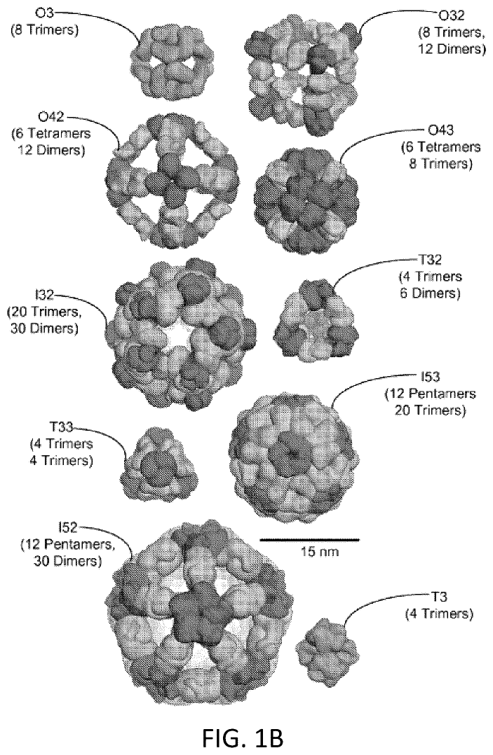

A non-limiting example of an embodiment is shown in FIG. 1A, which depicts an antigen genetically fused to a component of the protein nanostructure, which optionally is expressed recombinantly in a host cell (e.g., E. coli cells); along with a pentameric protein assembly as a second component, which is optionally expressed recombinantly in the same or a different host cell. These two components self-assembling into a protein nanostructure displaying 60 antigen monomers around an icosahedral core. In embodiments, an antigen is mixed with another antigen protein in the same protein nanostructure, such as two different variants of fHBP. In embodiments, the protein nanostructure comprises, in addition to one or more fHBP, antigens of other pathogenic organisms, and thus may be used as a combination vaccine. In embodiments, the protein nanostructure is further linked to polypeptides or other agents capable of acting as an adjuvant. In embodiments, the first component) and/or the second component comprise one or more T cell epitopes, optionally a T cell epitope of heterologous origin.

In embodiments, the first component is a trimeric component comprising three copies of the first polypeptide. In embodiments, the second component is a pentamer comprising five copies of the second polypeptide. In embodiments, the nanostructure comprises 20 copies of the first component. In embodiments, the nanostructure further comprises 12 copies of the second component.

In embodiments, three copies of a monomeric antigen are displayed on a 3-fold axis. Thus, the protein nanostructure depicted in FIG. 1A is capable of displaying 60 monomeric antigens (e.g., fHBP).

Other potential arrangements of components of the present disclosure are shown in FIG. 1B. In embodiments, the protein nanostructure is adapted for display of up to 12, 24, or 60 monomers. In embodiments a component may comprise polypeptide linked to diverse antigens, such that the protein nanostructure displays different antigens on the same nanostructure. In embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more different antigens are displayed. Non-limiting illustrative protein nanostructure are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

In embodiments the protein nanostructure is adapted to display the same antigen from two or more diverse strains of Neisseria meningitidis. In embodiments, the protein nanostructure displays an fHBP protein, or functional variant or antigenic fragment thereof, disclosed in any sequence of GenBank found by searching the Protein database with the keyword “Neisseria meningitidis factor H binding protein,” individually or in mixed protein nanostructures.

### Attachment Modalities

The protein nanostructures of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective.

In embodiments, attachment is achieved by genetic engineering to create an N- or C-terminal fusion of potentially antigenic polypeptides of the protein nanostructure.

In embodiments, attachment is achieved by post-translational covalent attachment of one or more pluralities of antigenic protein. In embodiments chemical cross-linking is used to non-specifically attach the antigen to a protein nanostructure. In embodiments chemical cross-linking is used to specifically attach the antigenic protein to a protein nanostructure (e.g. to the first polypeptide or the second polypeptide). Various specific and non-specific cross-linking chemistries are known in the art, such as Click chemistry and other methods. In general, any cross-linking chemistry/bioconjugate used to link two proteins may be adapted for use in the presently disclosed protein nanostructures. In particular, chemistries used in creation of immunoconjugates or antibody drug conjugates may be used. In embodiments a protein nanostructure is created using a cleavable or non-cleavable linker. Processes and methods for conjugation of antigens to carriers are provided by, e.g., Patent Pub. No. US 2008/0145373 A1.

In an embodiment, attachment is achieved by non-covalent attachment between a component and the antigen. In embodiments the antigenic protein is engineered to be negatively charged on at least one surface and the core polypeptide is engineered to be positively charged on at least one surface, or positively and negatively charged, respectively. This can promote intermolecular association between the antigenic protein and the component core polypeptide by electrostatic force. In embodiments shape complementarity is employed to cause linkage of antigen protein to component core. Shape complementarity can be pre-existing or rationally designed. In embodiments computational designed of protein-protein interfaces is used to achieve attachment. In an embodiment, the antigen is biotin-labeled and the polypeptide comprises a streptavidin, or vice versa. In an embodiment, streptavidin is displayed by gene fusion or otherwise as a tetramer on a 4-fold axis of the component core and the biotin-labeled antigen is monomeric, dimeric, or tetrameric, permitting association to the component core in a configuration appropriate for native multimerization of the antigen. In embodiments a protein-based adaptor is used to capture the antigenic protein. In embodiments the polypeptide is fused to a protein capable of binding a complementary protein, which is fused to the antigenic protein.

Immunogenicity of fHBP may be controlled by altering the orientation of the antigenic protein relative to the component core. Depending on how the antigenic protein is attached to the component core of the protein nanostructure, the antigenic protein may be displayed in various orientations. In embodiments, the antigenic protein is displayed so that one or more epitopes are oriented at or towards the distal end of the antigenic protein, such that these epitope(s) are preferentially accessible to the immune system. In embodiments the orientation will recapitulate the orientation of fHBP with respect to the bacterium. The choice of orientation may direct the immune system to one or the other epitope.

In embodiments, epitope preference is controlled by other means, such as positioning of glycans on the protein nanostructure by addition or subtraction of the N-linked glycan sequence motif N-X-[T/S] at predetermined positions in the amino acid sequence of a component including in the amino acid sequence of the antigenic protein.

In embodiments the epitopes found at intermediate distances from the proximal to the distal end will be the preferred over epitopes more distally located depending on various considerations including but not limited to the overall geometry of the protein nanostructure, surface hydrophobicity, surface charge, and competitive binding of proteins endogenously present in the subject or proteins exogenously provided in the vaccine composition. The present disclosure encompasses all known methods of rational design of protein structure and the foregoing is not intended to be limiting.

### Polypeptide Sequences

Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOS: 1-51 were designed for their ability to self-assemble in pairs to form protein nanostructures, such as icosahedral particles. The design involved design of suitable interface residues for each member of the polypeptide pair that can be assembled to form the protein nanostructure. The protein nanostructure so formed include symmetrically repeated, non-natural, non-covalent polypeptide-polypeptide interfaces that orient a first assembly and a second assembly into a protein nanostructure, such as one with an icosahedral symmetry. Thus, in one embodiment a first repeat element and second repeat element of the component are selected from the group consisting of SEQ ID NOS: 1-51. In each case, an N-terminal methionine residue present in the full-length protein is included, but may be removed to make a fusion that is not included in the sequence. The identified residues in Table 1 are numbered beginning with an N-terminal methionine (not shown). In various embodiments, one or more additional residues are deleted from the N-terminus and/or additional residues are added to the N-terminus (e.g. to form a helical extension).

TABLE 1

| | | | Identified |
|---|---|---|---|
| | Component | | interface |
| Name | Multimer | Amino Acid Sequence | residues |
| I53-34A | trimer | EGMDPLAVLAESRLLPLLTVRGGEDLAGLATVLELMGVGALE | I53-34A: |
| SEQ ID | | ITLRTEKGLEALKALRKSGLLLGAGTVRSPKEAEAALEAGAA | 28, 32, 36, |
| NO: 1 | | FLVSPGLLEEVAALAQARGVPYLPGVLTPTEVERALALGLSA | 37, 186, 188, |
| | | LKFFPAEPFQGVRVLRAYAEVFPEVRFLPTGGIKEEHLPHYA | 191, 192, 195 |
| | | ALPNLLAVGGSWLLQGDLAAVMKKVKAAKALLSPQAPG | |
| I53-34B | pentamer | TKKVGIVDTTFARVDMAEAAIRTLKALSPNIKIIRKTVPGIK | I53-34B: |
| SEQ ID | | DLPVACKKLLEEEGCDIVMALGMPGKAEKDKVCAHEASLGLM | 19, 20, 23, |
| NO: 2 | | LAQLMTNKHIIEVFVHEDEAKDDDELDILALVRAIEHAANVY | 24, 27, 109, |
| | | YLLFKPEYLTRMAGKGLRQGREDAGPARE | 113, 116, 117, |
| | | | 120, 124, 148 |
| I53-40A | pentamer | TKKVGIVDTTFARVDMASAAILTLKMESPNIKIIRKTVPGIK | I53-40A: |
| SEQ ID | | DLPVACKKLLEEEGCDIVMALGMPGKAEKDKVCAHEASLGLM | 20, 23, 24, |
| NO: 3 | | LAQLMTNKHIIEVFVHEDEAKDDAELKILAARRAIEHALNVY | 27, 28, 109, |
| | | YLLFKPEYLTRMAGKGLRQGFEDAGPARE | 112, 113, 116, |
| | | | 120, 124 |
| I53-40B | trimer | STINNQLKALKVIPVIAIDNAEDIIPLGKVLAENGLPAAEIT | I53-40B: |
| SEQ ID | | FRSSAAVKAIMLLRSAQPEMLIGAGTILNGVQALAAKEAGAT | 47, 51, 54, |
| NO: 4 | | FVVSPGFNPNTVRACQIIGIDIVPGVNNPSTVEAALEMGLTT | 58, 74, 102 |
| | | LKFFPAEASGGISMVKSLVGPYGDIRLMPTGGITPSNIDNYL | |
| | | AIPQVLACGGTWMVDKKLVTNGEWDEIARLTREIVEQVNP | |
| I53-47A | trimer | PIFTLNTNIKATDVPSDFLSLTSRLVGLILSKPGSYVAVHIN | I53-47A: |
| SEQ ID | | TDQQLSFGGSTNPAAFGTLMSIGGIEPSKNRDHSAVLFDHLN | 22, 25, 29, |
| NO: 5 | | AMLGIPKNRMYIHFVNLNGDDVGWNGTTF | 72, 79, 86, 87 |
| I53-47B | pentamer | NQHSHKDYETVRIAVVRARWHADIVDACVEAFEIAMAAIGGD | I53-47B: |
| SEQ ID | | RFAVDVEDVPGAYEIPLHARTLAETGRYGAVLGTAFVVNGGI | 28, 31, 35, |
| NO: 6 | | YRHEFVASAVIDGMMNVQLSTGVPVLSAVLTPHRYRDSAEHH | 36, 39, 131, |
| | | RFFAAHFAVKGVEAARACIEILAAREKIAA | 132, 135, 139, |
| | | | 146 |
| I53-50A | trimer | EELFKKHKIVAVLRANSVEEAIEKAVAVFAGGVHLIEITFTV | I53-50A: |
| SEQ ID | | PDADTVIKALSVLKEKGAIIGAGTVTSVEQCRKAVESGAEFI | 25, 29, 33, |
| NO: 7 | | VSPHLDEEISQFCKEKGVFYMPGVMTPTELVKAMKLGHTILK | 54, 57 |
| | | LFPGEVVGPQFVKAMKGPFPNVKFVPTGGVNLDNVCEWFKAG | |
| | | VLAVGVGSALVKGTPDEVREKAKAFVEKIRGCTE | |
| I53-50B | pentamer | NQHSHKDYETVRIAVVRARWHAEIVDACVSAFEAAMADIGGD | I53-50B: |
| SEQ ID | | RFAVDVFDVPGAYEIPLHARTLAETGRYGAVLGTAFVVNGGI | 24, 28, 36, |
| NO: 8 | | YRHEFVASAVIDGMMNVQLSTGVPVLSAVLTPHRYRDSDAHT | 124, 125, 127, |
| | | LLFLALFAVKGMEAARACVEILAAREKIAA | 128, 129, |
| | | | 131, 132, |
| | | | 133, 135, 139 |
| I53-51A | trimer | FTKSGDDGNTNVINKRVGKDSPLVNFLGDLDELNSFIGFAIS | I53-51A: |
| SEQ ID | | KIPWEDMKKDLERVQVELFEIGEDLSTQSSKKKIDESYVLWL | 80, 83, 86, |
| NO: 9 | | LAATAIYRIESGPVKLFVIPGGSEEASVLHVTRSVARRVERN | 87, 88, 90, |
| | | AVKYTKELPEINRMIIVYLNRLSSLLFAMALVANKRRNQSEK | 91, 94, 166, |
| | | IYEIGKSW | 172, 176 |
| I53-51B | pentamer | NQHSHKDYETVRIAVVRARWHADIVDQCVRAFEEAMADAGGD | I53-51B: |
| SEQ ID | | RFAVDVFDVPGAYEIPLHARTLAETGRYGAVLGTAFVVNGGI | 31, 35, 36, |
| NO: 10 | | YRHEFVASAVIDGMMNVQLSTGVPVLSAVLTPHRYRSSREHH | 40, 122, 124, |
| | | EFFREHFMVKGVEAAAACITILAAREKIAA | 128, 131, |
| | | | 135, 139, 143, |
| | | | 146, 147 |
| I52-03A | pentamer | GHTKGPTPQQHDGSALRIGIVHARWNKTIIMPLLIGTIAKLL | I52-03A: |
| SEQ ID | | ECGVKASNIVVQSVPGSWELPIAVQRLYSASQLQTPSSGPSL | 28, 32, 36, |
| NO: 11 | | SAGDLLGSSTTDLTALPTTTASSTGPFDALIAIGVLIKGETM | 39, 44, 49 |
| | | HFEYIADSVSHGLMRVQLDTGVPVIFGVLTVLTDDQAKARAG | |
| | | VIEGSHNHGEDWGLAAVEMGVRRRDWAAGKTE | |
| I52-03B | dimer | YEVDHADVYDLFYLGRGKDYAAEASDIADLVRSRTPEASSLL | I52-03B: |
| SEQ ID | | DVACGTGTHLEHFTKEFGDTAGLELSEDMLTHARKRLPDATL | 94, 115, 116, |
| NO: 12 | | HQGDMRDFQLGRKFSAVVSMFSSVGYLKTVAELGAAVASFAE | 206, 213 |
| | | HLEPGGVVVVEPWWFPETFADGWVSADVVRRDGRTVARVSHS | |
| | | VREGNATRMEVHFTVADPGKGVRHFSDVHLITLFHQREYEAA | |
| | | FMAAGLRVEYLEGGPSGRGLFVGVPA | |
| I52-32A | dimer | GMKEKFVLIITHGDFGKGLLSGAEVIIGKQENVHTVGLNLGD | I52-32A: |
| SEQ ID | | NIEKVAKEVMRIIIAKLAEDKEIIIVVDLFGGSPENIALEMM | 47, 49, 53, |
| NO: 13 | | KTFDVKVITGINMPMLVELLTSINVYDTTELLENISKIGKDG | 54, 57, 58, 61, |
| | | IKVIEKSSLKM | 83, 87, 88 |
| I52-32B | pentamer | KYDGSKLRIGILHARWNLEIIAALVAGAIKRLQEFGVKAENI | I52-32B: |
| SEQ ID | | IIETVPGSFELPYGSKLFVEKQKRLGKPLDAIIPIGVLIKGS | 19, 20, 23, |
| NO: 14 | | TMHFEYICDSTTHQLMKLNFELGIPVIFGVLTCLTDEQAEAR | 30, 40 |
| | | AGLIEGKMHNHGEDWGAAAVEMATKFN | |
| I52-33A | pentamer | AVKGLGEVDQKYDGSKLRIGILHARWNRKIILALVAGAVLRL | I52-33A: |
| SEQ ID | | LEFGVKAENIIIETVPGSFELPYGSKLFVEKQKRLGKPLDAI | 33, 41, 44, |
| NO: 15 | | IPIGVLIKGSTMHFEYICDSTTHQLMKLNFELGIPVIFGVLT | 50 |
| | | CLTDEQAEARAGLIEGKMHNHGEDWGAAAVEMATKFN | |
| I52-33B | dimer | GANWYLDNESSRLSFTSTKNADIAEVHRFLVLHGKVDPKGLA | I52-33B: |
| SEQ ID | | EVEVETESISTGIPLRDMLLRVLVFQVSKFPVAQINAQLDMR | 61, 63, 66, |
| NO: 16 | | PINNLAPGAQLELRLPLTVSLRGKSHSYNAELLATRLDERRF | 67, 72, 147, |
| | | QVVTLEPLVIHAQDFDMVRAFNALRLVAGLSAVSLSVPVGAV | 148, 154, 155 |
| | | LIFTAR | |
| I32-06A | dimer | TDYIRDGSAIKALSFAIILAEADLRHIPQDLQRLAVRVIHAC | I32-06A: |
| SEQ ID | | GMVDVANDLAFSEGAGKAGRNALLAGAPILCDARMVAEGITR | 9, 12, 13, 14, |
| NO: 17 | | SRLPADNRVIYTLSDPSVPELAKKIGNTRSAAALDLWLPHIE | 20, 30, 33, |
| | | GSIVAIGNAPTALFRLFELLDAGAPKPALIIGMPVGFVGAAE | 34 |
| | | SKDELAANSRGVPYVIVRGRRGGSAMTAAAVNALASERE | |
| I32-06B | trimer | ITVFGLKSKLAPRREKLAEVIYSSLHLGLDIPKGKHAIRFLC | I32-06B: |
| SEQ ID | | LEKEDFYYPFDRSDDYTVIEINLMAGRSEETKMLLIFLLFIA | 24, 71, 73, |
| NO: 18 | | LERKLGIRAHDVEITIKEQPAHCWGFRGRTGDSARDLDYDIY | 76, 77, 80, 81, |
| | | V | 84, 85, 88, |
| | | | 114, 118 |
| I32-19A | trimer | GSDLQKLQRFSTCDISDGLLNVYNIPTGGYFPNLTAISPPQN | I32-19A: |
| SEQ ID | | SSIVGTAYTVLFAPIDDPRPAVNYIDSVPPNSILVLALEPHL | 208, 213, 218, |
| NO: 19 | | QSQFHPFIKITQAMYGGLMSTRAQYLKSNGTVVFGRIRDVDE | 8222, 225, |
| | | HRTLNHPVFAYGVGSCAPKAVVKAVGTNVQLKILTSDGVTQT | 226, 229, 233 |
| | | ICPGDYIAGDNNGIVRIPVQETDISKLVTYIEKSIEVDRLVS | |
| | | EAIKNGLPAKAAQTARRMVLKDYI | |
| I32-19B | dimer | SGMRVYLGADHAGYELKQAIIAFLKMTGHEPIDCGALRYDAD | I32-19B: |
| SEQ ID | | DDYPAFCIAAATRTVADPGSLGIVLGGSGNGEQIAANKVPGA | 20, 23, 24, 27, |
| NO: 20 | | RCALAWSVQTAALAREHNNAQLIGIGGRMHTLEEALRIVKAF | 117, 118, |
| | | VTTPWSKAQRHQRRIDILAEYERTHEAPPVPGAPA | 122, 125 |
| I32-28A | trimer | GDDARIAAIGDVDELNSQIGVLLAEPLPDDVRAALSAIQHDL | I32-28A: |
| SEQ ID | | FDLGGELCIPGHAAITEDHLLRLALWLVHYNGQLPPLEEFIL | 60, 61, 64, 67, |
| NO: 21 | | PGGARGAALAHVCRTVCRRAERSIKALGASEPLNIAPAAYVN | 68, 71, 110, |
| | | LLSDLLFVLARVLNRAAGGADVLWDRTRAH | 120, 123, |
| | | | 124, 128 |
| I32-28B | dimer | ILSAEQSFTLRHPHGQAAALAFVREPAAALAGVQRLRGLDSD | I32-28B: |
| SEQ ID | | GEQVWGELLVRVPLLGEVDLPFRSEIVRTPQGAELRPLTLTG | 35, 36, 54, |
| NO: 22 | | ERAWVAVSGQATAAEGGEMAFAFQFQAHLATPEAEGEGGAAF | 122, 129, 137, |
| | | EVMVQAAAGVTLLLVAMALPQGLAAGLPPA | 140, 141, 144, |
| | | | 148 |
| I53-40A.1 | pentamer | TKKVGIVDTTFARVDMASAAILTLKMESPNIKIIRKTVPGIK | I53-40A: |
| SEQ ID | | DLPVACKKLLEEEGCDIVMALGMPGKKEKDKVCAHEASLGLM | 20, 23, 24, 27, |
| NO: 23 | | LAQLMTNKHIIEVFVHEDEAKDDAELKILAARRAIEHALNVY | 28, 109, 112, |
| | | YLLFKPEYLTRMAGKGLRQGFEDAGPARE | 113, 116, |
| | | | 120, 124 |
| I53-40B.1 | trimer | DDINNQLKRLKVIPVIAIDNAEDIIPLGKVLAENGLPAAEIT | I53-40B: |
| SEQ ID | | FRSSAAVKAIMLLRSAQPEMLIGAGTILNGVQALAAKEAGAD | 47, 51, 54, 58, |
| NO: 24 | | FVVSPGFNPNTVRACQIIGIDIVPGVNNPSTVEQALEMGLTT | 74, 102 |
| | | LKFFPAEASGGISMVKSLVGPYGDIRLMPTGGITPDNIDNYL | |
| | | AIPQVLACGGTWMVDKKLVRNGEWDEIARLTREIVEQVNP | |
| I53-47A.1 | trimer | PIFTLNTNIKADDVPSDFLSLTSRLVGLILSKPGSYVAVHIN | I53-47A: |
| SEQ ID | | TDQQLSFGGSTNPAAFGTLMSIGGIEPDKNRDHSAVLFDHLN | 22, 25, 29, 72, |
| NO: 25 | | AMLGIPKNRMYIHFVNLNGDDVGWNGTTF | 79, 86, 87 |
| I53- | trimer | PIFTLNTNIKADDVPSDFLSLTSRLVGLILSEPGSYVAVHIN | I53-47A: |
| 47A.1NegT | | TDQQLSFGGSTNPAAFGTLMSIGGIEPDKNEDHSAVLFDHLN | 22, 25, 29, 72, |
| 2 | | AMLGIPKNRMYIHFVDLDGDDVGWNGTTF | 79, 86, 87 |
| SEQ ID | | | |
| NO: 26 | | | |
| I53-47B.1 | pentamer | NQHSHKDHETVRIAVVRARWHADIVDACVEAFEIAMAAIGGD | I53-47B: |
| SEQ ID | | RFAVDVFDVPGAYEIPLHARTLAETGRYGAVLGTAFVVNGGI | 28, 31, 35, 36, |
| NO: 27 | | YRHEFVASAVIDGMMNVQLDTGVPVLSAVLTPHRYRDSDEHH | 39, 131, 132, |
| | | RFFAAHFAVKGVEAARACIEILNAREKIAA | 135, 139, 146 |
| I53- | pentamer | NQHSHKDHETVRIAVVRARWHADIVDACVEAFEIAMAAIGGD | I53-47B: |
| 47B.1NegT | | RFAVDVFDVPGAYEIPLHARTLAETGRYGAVLGTAFVVDGGI | 28, 31, 35, 36, |
| 2 | | YDHEFVASAVIDGMMNVQLDTGVPVLSAVLTPHEYEDSDEDH | 39, 131, 132, |
| SEQ ID | | EFFAAHFAVKGVEAARACIEILNAREKIAA | 135, 139, 146 |
| NO: 28 | | | |
| I53-50A.1 | trimer | EELFKKHKIVAVLRANSVEEAIEKAVAVFAGGVHLIEITFTV | I53-50A: |
| SEQ ID | | PDADTVIKALSVLKEKGAIIGAGTVTSVEQCRKAVESGAEFI | 25, 29, 33, |
| NO: 29 | | VSPHLDEEISQFCKEKGVFYMPGVMTPTELVKAMKLGHDILK | 54, 57 |
| | | LFPGEVVGPQFVKAMKGPFPNVKFVPTGGVNLDNVCEWFKAG | |
| | | VLAVGVGDALVKGDPDEVREKAKKFVEKIRGCTE | |
| I53- | trimer | EELFKKHKIVAVLRANSVEEAIEKAVAVFAGGVHLIEITFTV | I53-50A: |
| 50A.1NegT | | PDADTVIKALSVLKEKGAIIGAGTVTSVEQCRKAVESGAEFI | 25, 29, 33, |
| 2 | | VSPHLDEEISQFCKEKGVFYMPGVMTPTELVKAMKLGHDILK | 54, 57 |
| SEQ ID | | LFPGEVVGPEFVEAMKGPFPNVKFVPTGGVDLDDVCEWFDAG | |
| NO: 30 | | VLAVGVGDALVEGDPDEVREDAKEFVEEIRGCTE | |
| I53- | trimer | EELFKKHKIVAVLRANSVEEAIEKAVAVFAGGVHLIEITFTV | I53-50A: |
| 50A.1PosT | | PDADTVIKALSVLKEKGAIIGAGTVTSVEQCRKAVESGAEFI | 25, 29, 33, |
| 1 | | VSPHLDEEISQFCKEKGVFYMPGVMTPTELVKAMKLGHDILK | 54, 57 |
| SEQ ID | | LFPGEVVGPQFVKAMKGPFPNVKFVPTGGVNLDNVCKWFKAG | |
| NO: 31 | | VLAVGVGKALVKGKPDEVREKAKKFVKKIRGCTE | |
| I53-50B.1 | pentamer | NQHSHKDHETVRIAVVRARWHAEIVDACVSAFEAAMRDIGGD | I53-50B: |
| SEQ ID | | RFAVDVFDVPGAYEIPLHARTLAETGRYGAVLGTAFVVNGGI | 24, 28, 36, |
| NO: 32 | | YRHEFVASAVIDGMMNVQLDTGVPVLSAVLTPHRYRDSDAHT | 124, 125, 127, |
| | | LLFLALFAVKGMEAARACVEILAAREKIAA | 128, 129, 131, |
| | | | 132, 133, |
| | | | 135, 139 |
| I53- | pentamer | NQHSHKDHETVRIAVVRARWHAEIVDACVSAFEAAMRDIGGD | I53-50B: |
| 50B.1NegT | | RFAVDVFDVPGAYEIPLHARTLAETGRYGAVLGTAFVVDGGI | 24, 28, 36, |
| 2 | | YDHEFVASAVIDGMMNVQLDTGVPVLSAVLTPHEYEDSDADT | 124, 125, 127, |
| SEQ ID | | LLFLALFAVKGMEAARACVEILAAREKIAA | 128, 129, 131, |
| NO: 33 | | | 132, 133, |
| | | | 135, 139 |
| I53- | trimer | NQHSHKDHETVRIAVVRARWHAEIVDACVSAFEAAMRDIGGD | I53-50B: |
| 50B.4POST | | RFAVDVFDVPGAYEIPLHARTLAETGRYGAVLGTAFVVNGGI | 24, 28, 36, |
| 1 | | YRHEFVASAVINGMMNVQLNTGVPVLSAVLTPHNYDKSKAHT | 124, 125, 127, |
| SEQ ID | | LLFLALFAVKGMEAARACVEILAAREKIAA | 128, 129, 131, |
| NO: 34 | | | 132, 133, |
| | | | 135, 139 |
| I53-40A | pentamer | TKKVGIVDTTFARVDMASAAILTLKMESPNIKIIRKTVPGIK | |
| genus | | DLPVACKKLLEEEGCDIVMALGMPGK(A/K)EKDKVCAHEAS | |
| SEQ ID | | LGLMLAQLMTNKHIIEVFVHEDEAKDDAELKILAARRAIEHA | |
| NO: 35 | | LNVYYLLFKPEYLTRMAGKGLRQGFEDAGPARE | |
| I53-40B | trimer | (S/D)(T/D)INNQLK(A/R)LKVIPVIAIDNAEDIIPLGKV | |
| genus | | LAENGLPAAEITFRSSAAVKAIMLLRSAQPEMLIGAGTILNG | |
| SEQ ID | | VQALAAKEAGA(T/D)FVVSPGFNPNTVRACQIIGIDIVPGV | |
| NO: 36 | | NNPSTVE(A/Q)ALEMGLTTLKFFPAEASGGISMVKSLVGPY | |
| | | GDIRLMPTGGITP(S/D)NIDNYLAIPQVLACGGTWMVDKKL | |
| | | V(T/R)NGEWDEIARLTREIVEQVNP | |
| I53-47A | trimer | PIFTLNTNIKA(T/D)DVPSDFLSLTSRLVGLILS(K/E)PG | |
| genus | | SYVAVHINTDQQLSFGGSTNPAAFGTLMSIGGIEP(S/D)KN | |
| SEQ ID | | (R/E)DHSAVLFDHLNAMLGIPKNRMYIHFV(N/D)L(N/D) | |
| NO: 37 | | GDDVGWNGTTF | |
| I53-47B | pentamer | NQHSHKD(Y/H)ETVRIAVVRARWHADIVDACVEAFEIAMAA | |
| genus | | IGGDRFAVDVFDVPGAYEIPLHARTLAETGRYGAVLGTAFVV | |
| SEQ ID | | (N/D)GGIY(R/D)HEFVASAVIDGMMNVQL(S/D)TGVPVL | |
| NO: 38 | | SAVLTPH(R/E)Y(R/E)DS(A/D)E(H/D)H(R/E)FFAAH | |
| | | FAVKGVEAARACIEIL(A/N)AREKIAA | |
| I53-50A | trimer | EELFKKHKIVAVLRANSVEEAIEKAVAVFAGGVHLIEITFTV | |
| genus | | PDADTVIKALSVLKEKGAIIGAGTVTSVEQCRKAVESGAEFI | |
| SEQ ID | | VSPHLDEEISQFCKEKGVFYMPGVMTPTELVKAMKLGH(T/D) | |
| NO: 39 | | ILKLFPGEVVGP(Q/E)FV(K/E)AMKGPFPNVKFVPTGGV | |
| | | (N/D)LD(N/D)VC(E/K)WF(K/D)AGVLAVGVG(S/K/D) | |
| | | ALV(K/E)G(T/D/K)PDEVRE(K/D)AK(A/E/K)FV(E/K) | |
| | | (K/E)IRGCTE | |
| I53-50B | pentamer | NQHSHKD(Y/H)ETVRIAVVRARWHAEIVDACVSAFEAAM(A/ | |
| genus | | R)DIGGDRFAVDVFDVPGAYEIPLHARTLAETGRYGAVLGT | |
| SEQ ID | | AFVV(N/D)GGIY(R/D)HEFVASAVI(D/N)GMMNVQL(S/ | |
| NO: 40 | | D/N)TGVPVLSAVLTPH(R/E/N)Y(R/D/E)(D/K)S(D/K) | |
| | | A(H/D)TLLFLALFAVKGMEAARACVEILAAREKIAA | |
| T32-28A | dimer | GEVPIGDPKELNGMEIAAVYLQPIEMEPRGIDLAASLADIHL | |
| SEQ ID | | EADIHALKNNPNGFPEGFWMPYLTIAYALANADTGAIKTGTL | |
| NO: 41 | | MPMVADDGPHYGANIAMEKDKKGGFGVGTYALTFLISNPEKQ | |
| | | GFGRHVDEETGVGKWFEPFVVTYFFKYTGTPK | |
| T32-28B | trimer | SQAIGILELTSIAKGMELGDAMLKSANVDLLVSKTISPGKFL | |
| SEQ ID | | LMLGGDIGAIQQAIETGTSQAGEMLVDSLVLANIHPSVLPAI | |
| NO: 42 | | SGLNSVDKRQAVGIVETWSVAACISAADLAVKGSNVTLVRVH | |
| | | MAFGIGGKCYMVVAGDVLDVAAAVATASLAAGAKGLLVYASI | |
| | | IPRPHEAMWRQMVEG | |
| T33-09A | trimer | EEVVLITVPSALVAVKIAHALVEERLAACVNIVPGLTSIYRW | |
| SEQ ID | | QGSVVSDHELLLLVKTTTHAFPKLKERVKALHPYTVPEIVAL | |
| NO: 43 | | PIAEGNREYLDWLRENTG | |
| T33-09B | trimer | VRGIRGAITVEEDTPAAILAATIELLLKMLEANGIQSYEELA | |
| SEQ ID | | AVIFTVTEDLTSAFPAEAARLIGMHRVPLLSAREVPVPGSLP | |
| NO: 44 | | RVIRVLALWNTDTPQDRVRHVYLNEAVRLRPDLESAQ | |
| T33-15A | trimer | SKAKIGIVTVSDRASAGITADISGKAIILALNLYLTSEWEPI | |
| SEQ ID | | YQVIPDEQDVIETTLIKMADEQDCCLIVTTGGTGPAKRDVTP | |
| NO: 45 | | EATEAVCDRMMPGFGELMRAESLKEVPTAILSRQTAGLRGDS | |
| | | LIVNLPGDPASISDCLLAVFPAIPYCIDLMEGPYLECNEAMI | |
| | | KPFRPKAK | |
| T33-15B | trimer | VRGIRGAITVNSDTPTSIIIATILLLEKMLEANGIQSYEELA | |
| SEQ ID | | AVIFTVTEDLTSAFPAEAARQIGMHRVPLLSAREVPVPGSLP | |
| NO: 46 | | RVIRVLALWNTDTPQDRVRHVYLSEAVRLRPDLESAQ | |
| T33-21A | trimer | RITTKVGDKGSTRLFGGEEVWKDSPIIEANGTLDELTSFIGE | |
| SEQ ID | | AKHYVDEEMKGILEEIQNDIYKIMGEIGSKGKIEGISEERIA | |
| NO: 47 | | WLLKLILRYMEMVNLKSFVLPGGTLESAKLDVCRTIARRALR | |
| | | KVLTVTREFGIGAEAAAYLLALSDLLFLLARVIEIEKNKLKE | |
| | | VRS | |
| T33-21B | trimer | PHLVIEATANLRLETSPGELLEQANKALFASGQFGEADIKSR | |
| SEQ ID | | FVTLEAYRQGTAAVERAYLHACLSILDGRDIATRTLLGASLC | |
| NO: 48 | | AVLAEAVAGGGEEGVQVSVEVREMERLSYAKRVVARQR | |
| T33-28A | trimer | ESVNTSFLSPSLVTIRDFDNGQFAVLRIGRTGFPADKGDIDL | |
| SEQ ID | | CLDKMIGVRAAQIFLGDDTEDGFKGPHIRIRCVDIDDKHTYN | |
| NO: 49 | | AMVYVDLIVGTGASEVERETAEEEAKLALRVALQVDIADEHS | |
| | | CVTQFEMKLREELLSSDSFHPDKDEYYKDFL | |
| T33-28B | trimer | PVIQTFVSTPLDHHKRLLLAIIYRIVTRVVLGKPEDLVMMTF | |
| SEQ ID | | HDSTPMHFFGSTDPVACVRVEALGGYGPSEPEKVTSIVTAAI | |
| NO: 50 | | TAVCGIVADRIFVLYFSPLHCGWNGTNF | |
| T33-31A | trimer | EEVVLITVPSALVAVKIAHALVEERLAACVNIVPGLTSTYRE | |
| SEQ ID | | EGSVVSDHELLLLVKTTTDAFPKLKERVKELHPYEVPEIVAL | |
| NO: 51 | | PIAEGNREYLDWLRENTG | |
| I53-50A | trimer | EELFKKHKIVAVLRANSVEEAIEKAVAVFAGGVHLIEITFTV | |
| ΔCys | | PDADTVIKALSVLKEKGAIIGAGTVTSVEQARKAVESGAEFI | |
| SEQ ID | | VSPHLDEEISQFAKEKGVFYMPGVMTPTELVKAMKLGHTILK | |
| NO: 53 | | LFPGEVVGPQFVKAMKGPFPNVKFVPTGGVNLDNVAEWFKAG | |
| | | VLAVGVGSALVKGTPDEVREKAKAFVEKIRGATE | |
| T33_dn2A | | NLAEKMYKAGNAMYRKGQYTIAIIAYTLALLKDPNNAEAWYN | |
| SEQ ID | | LGNAAYKKGEYDEAIEAYQKALELDPNNAEAWYNLGNAYYKQ | |
| NO: 135 | | GDYDEAIEYYKKALRLDPRNVDAIENLIEAEEKQG | |
| T33_dn2B | | EEAELAYLLGELAYKLGEYRIAIRAYRIALKRDPNNAEAWYN | |
| SEQ ID | | LGNAYYKQGDYREAIRYYLRALKLDPENAEAWYNLGNALYKQ | |
| NO: 136 | | GKYDLAIIAYQAALEEDPNNAEAKQNLGNAKQKQG | |
| T33_dn5A | | NSAEAMYKMGNAAYKQGDYILAIIAYLLALEKDPNNAEAWYN | |
| SEQ ID | | LGNAAYKQGDYDEAIEYYQKALELDPNNAEAWYNLGNAYYKQ | |
| NO: 137 | | GDYDEAIEYYEKALELDPNNAEALKNLLEAIAEQD | |
| T33_dn5A | | TDPLAVILYIAILKAEKSIARAKAAEALGKIGDERAVEPLIK | |
| SEQ ID | | ALKDEDALVRAAAADALGQIGDERAVEPLIKALKDEEGLVRA | |
| NO: 138 | | SAAIALGQIGDERAVQPLIKALTDERDLVRVAAAVALGRIGD | |
| | | EKAVRPLIIVLKDEEGEVREAAAIALGSIGGERVRAAMEKLA | |
| | | ERGTGFARKVAVNYLETHK | |
| T33_dn10A | | EEAELAYLLGELAYKLGEYRIAIRAYRIALKRDPNNAEAWYN | |
| SEQ ID | | LGNAYYKQGDYDEAIEYYQKALELDPNNAEAWYNLGNAYYKQ | |
| NO: 139 | | GDYDEAIEYYEKALELDPENLEALQNLLNAMDKQG | |
| T33_dn10B | | IEEVVAEMIDILAESSKKSIEELARAADNKTTEKAVAEAIEE | |
| SEQ ID | | IARLATAAIQLIEALAKNLASEEFMARAISAIAELAKKAIEA | |
| NO: 140 | | IYRLADNHTTDTFMARAIAAIANLAVTAILAIAALASNHTTE | |
| | | EFMARAISAIAELAKKAIEAIYRLADNHTTDKFMAAAIEAIA | |
| | | LLATLAILAIALLASNHTTEKFMARAIMAIAILAAKAIEAIY | |
| | | RLADNHTSPTYIEKAIEAIEKIARKAIKAIEMLAKNITTEEY | |
| | | KEKAKKIIDIIRKLAKMAIKKLEDNRT | |
| I53_dn5A | pentamer | KYDGSKLRIGILHARWNAEIILALVLGALKRLQEFGVKRENI | |
| SEQ ID | | IIETVPGSFELPYGSKLFVEKQKRLGKPLDAIIPIGVLIKGS | |
| NO: 141 | | TMHFEYICDSTTHQLMKLNFELGIPVIFGVLTCLTDEQAEAR | |
| | | AGLIEGKMHNHGEDWGAAAVEMATKFN | |
| I53 dn5B | trimer | EEAELAYLLGELAYKLGEYRIAIRAYRIALKRDPNNAEAWYN | |
| SEQ ID | | LGNAYYKQGRYREAIEYYQKALELDPNNAEAWYNLGNAYYER | |
| NO: 142 | | GEYEEAIEYYRKALRLDPNNADAMQNLLNAKMREE | |
| I53 dn5A. | pentamer | KYDGSKLRIGILHARGNAEIILALVLGALKRLQEFGVKRENI | |
| 1 SEQ ID | | IIETVPGSFELPYGSKLFVEKQKRLGKPLDAIIPIGVLIRGS | |
| NO: 133 | | TPHFDYIADSTTHQLMKLNFELGIPVIFGVITADTDEQAEAR | |
| | | AGLIEGKMHNHGEDWGAAAVEMATKFN | |
| I53_dn5A. | pentamer | KYDGSKLRIGILHARGNAEIILELVLGALKRLQEFGVKRENI | |
| 2 SEQ ID | | IIETVPGSFELPYGSKLFVEKQKRLGKPLDAIIPIGVLIRGS | |
| NO: 134 | | TAHFDYIADSTTHQLMKLNFELGIPVIFGVLTTESDEQAEER | |
| | | AGTKAGNHGEDWGAAAVEMATKFN | |

Table 1 provides the amino acid sequence of a first repeat element and second repeat element of embodiments of the present disclosure. In each case, the pairs of sequences together form an I53 multimer with icosahedral symmetry. The right-hand column in Table 1 identifies the residue numbers in each illustrative polypeptide that were identified as present at the interface of resulting assembled protein nanostructures (i.e.: “identified interface residues”). As can be seen, the number of interface residues for the illustrative polypeptides of SEQ ID NO: 1-34 range from 4-13. In various embodiments, a first repeat element and second repeat element comprise an amino acid sequence that is at least 75%, 80%, 85%, 90%, 91%, 92%, 93%, 94%, 95%, 96%, 97%, 98%, or 99% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. SEQ ID NOs: 35-51 represent other amino acid sequences of a first repeat element and second repeat element from embodiments of the present disclosure. In other embodiments, a first repeat element and/or second repeat element comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%, 60%, 70%, 75%, 80%, 90%, or 100% of the identified interface positions, to the amino acid sequence of a polypeptide selected from the group consisting of SEQ ID NOS: 1-51.

As is the case with proteins in general, the polypeptides are expected to tolerate some variation in the designed sequences without disrupting subsequent assembly into protein nanostructures: particularly when such variation comprises conservative amino acid substitutions. As used here, “conservative amino acid substitution” means that: hydrophobic amino acids (Ala, Cys, Gly, Pro, Met, Val, Ile, Leu) are substituted with other hydrophobic amino acids; hydrophobic amino acids with bulky side chains (Phe, Tyr, Trp) are substituted with other hydrophobic amino acids with bulky side chains; amino acids with positively charged side chains (Arg, His, Lys) are substituted with other amino acids with positively charged side chains; amino acids with negatively charged side chains (Asp, Glu) are substituted with other amino acids with negatively charged side chains; and amino acids with polar uncharged side chains (Ser, Thr, Asn, Gln) are substituted with other amino acids with polar uncharged side chains.

In various embodiments of the protein nanostructures of the invention, a first repeat element and second repeat element, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 95%, at least 96%, at least 97%, at least 98%, at least 99%, at least 100% over its length, and/or identical at least at one identified interface position, to the amino acid sequence indicated by the SEQ ID NO):
SEQ ID NO:1 and SEQ ID NO:2 (I53-34A and I53-34B);
SEQ ID NO:3 and SEQ ID NO:4 (I53-40A and I53-40B);
SEQ ID NO:3 and SEQ ID NO:24 (I53-40A and I53-40B.1);
SEQ ID NO:23 and SEQ ID NO:4 (I53-40A.1 and I53-40B);
SEQ ID NO:35 and SEQ ID NO:36 (I53-40A genus and I53-40B genus);
SEQ ID NO:5 and SEQ ID NO:6 (I53-47A and I53-47B);
SEQ ID NO:5 and SEQ ID NO:27 (I53-47A and I53-47B.1);
SEQ ID NO:5 and SEQ ID NO:28 (I53-47A and I53-47B.1NegT2);
SEQ ID NO:25 and SEQ ID NO:6 (I53-47A.1 and I53-47B);
SEQ ID NO:25 and SEQ ID NO:27 (I53-47A.1 and I53-47B.1);
SEQ ID NO:25 and SEQ ID NO:28 (I53-47A.1 and I53-47B.1NegT2);
SEQ ID NO:26 and SEQ ID NO:6 (I53-47A.1NegT2 and I53-47B);
SEQ ID NO:26 and SEQ ID NO:27 (I53-47A.1NegT2 and I53-47B.1);
SEQ ID NO:26 and SEQ ID NO:28 (I53-47A.1NegT2 and I53-47B.1NegT2);
SEQ ID NO:37 and SEQ ID NO:38 (I53-47A genus and I53-47B genus);
SEQ ID NO:7 and SEQ ID NO:8 (I53-50A and I53-50B);
SEQ ID NO:7 and SEQ ID NO:32 (I53-50A and I53-50B.1);
SEQ ID NO:7 and SEQ ID NO:33 (I53-50A and I53-50B.1NegT2);
SEQ ID NO:7 and SEQ ID NO:34 (I53-50A and I53-50B.4PosT1);
SEQ ID NO:29 and SEQ ID NO:8 (I53-50A.1 and I53-50B);
SEQ ID NO:29 and SEQ ID NO:32 (I53-50A.1 and I53-50B.1);
SEQ ID NO:29 and SEQ ID NO:33 (I53-50A.1 and I53-50B.1NegT2);
SEQ ID NO:29 and SEQ ID NO:34 (I53-50A.1 and I53-50B.4PosT1);
SEQ ID NO:30 and SEQ ID NO:8 (I53-50A.1NegT2 and I53-50B);
SEQ ID NO:30 and SEQ ID NO:32 (I53-50A.1NegT2 and I53-50B.1);
SEQ ID NO:30 and SEQ ID NO:33 (I53-50A.1NegT2 and I53-50B.1NegT2);
SEQ ID NO:30 and SEQ ID NO:34 (I53-50A.1 NegT2 and I53-50B.4PosT1);
SEQ ID NO:31 and SEQ ID NO:8 (I53-50A.1PosT1 and I53-50B);
SEQ ID NO:31 and SEQ ID NO:32 (I53-50A.1PosT1 and I53-50B.1);
SEQ ID NO:31 and SEQ ID NO:33 (I53-50A.1PosT1 and I53-50B.1NegT2);
SEQ ID NO:31 and SEQ ID NO:34 (I53-50A.1PosT1 and I53-50B.4PosT1);
SEQ ID NO:39 and SEQ ID NO:40 (I53-50A genus and I53-50B genus);
SEQ ID NO:9 and SEQ ID NO: 10 (I53-51A and I53-51B);
SEQ ID NO:11 and SEQ ID NO:12 (152-03A and I52-03B);
SEQ ID NO:13 and SEQ ID NO:14 (152-32A and I52-32B);
SEQ ID NO:15 and SEQ ID NO:16 (152-33A and I52-33B)
SEQ ID NO:17 and SEQ ID NO:18 (I32-06A and I32-06B);
SEQ ID NO:19 and SEQ ID NO:20 (I32-19A and I32-19B);
SEQ ID NO:21 and SEQ ID NO:22 (I32-28A and I32-28B);
SEQ ID NO:23 and SEQ ID NO:24 (I53-40A.1 and I53-40B.1);
SEQ ID NO:41 and SEQ ID NO:42 (T32-28A and T32-28B);
SEQ ID NO:43 and SEQ ID NO:44 (T33-09A and T33-09B);
SEQ ID NO:45 and SEQ ID NO:46 (T33-15A and T33-15B);
SEQ ID NO:47 and SEQ ID NO:48 (T33-21A and T33-21B);
SEQ ID NO:49 and SEQ ID NO:50 (T33-28A and T32-28B); and
SEQ ID NO:51 and SEQ ID NO:44 (T33-31A and T33-09B (also referred to as T33-31B)).

In some embodiments, the assembly domains are I53_dn5B (trimer, optionally linked to the fHBP) and I53_dn5A or I53_dn5A.1 or I53_dn5A.2 (pentamer). I53_dn5 nanostructures are described in US 2022/0072120 A1, the contents of which are incorporated by reference. I53_dn5 variants may include one or more amino acid substitutions, such as C94A, C119A, W18G, K84R, M88P, E91D, L117I, or L120D (together “I53_dn5A.1”; Ueda et al. eLife 9: e57659 (2020) or A25E, M88A, C119T, L120E, A127E, L131T, 1132K, E133A, or a deletion of positions 135-137 (“I53_dn5A.2”; Wang et al. bioRxiv 2022.08.04.502842).

In embodiments, the one or more fHBPs, or antigenic fragments thereof, are expressed as a fusion protein with a first assembly domain. In embodiments, the first assembly domain and the fHBP are joined by a linker sequence.

Non-limiting examples of designed protein complexes useful in protein nanostructures of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety.

In various embodiments of the protein nanostructures of the disclosure, the assembly domains are polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 95%, at least 96%, at least 97%, at least 98%, at least 99%, at least 100% over its length, and/or identical at least at one identified interface position, to the amino acid sequence indicated by the SEQ ID NO):
SEQ ID NO: 135 and SEQ ID NO: 136 (T33_dn2A and T33_dn2B);
SEQ ID NO: 137 and SEQ ID NO: 138 (T33_dn5A and T33_dn5B);
SEQ ID NO: 139 and SEQ ID NO: 140 (T33_dn10A and T33_dn10B); or
SEQ ID NO: 141 and SEQ ID NO: 142 (I53_dn5A and I53_dn5B).

### Antigenic Proteins

The present disclosure provides protein nanostructure vaccines for bacterial meningitis. The present disclosure relates to incorporation of any antigenic fragment of fHBP—e.g., the protein or an antigenic fragment thereof—into protein nanostructure vaccines. Guidance is particularly available from studies of the immune response to infection or vaccination, such as isolation of binding or neutralizing antibodies, genetic analysis of fHBP sequence, structural studies of antigenic proteins and antibodies, and most particularly clinical and veterinary experience with subunit vaccines. Subunit vaccine for bacterial meningitis can be adapted for use with the protein nanostructures of the present disclosure by employing the display modalities provided above.

fHBP binds to human factor H, an inhibitor of the alternative complement pathway. Evasion of the human complement system is critical for meningococci to cause invasive disease. The observation that persons deficient in various complement components are highly predisposed to invasive meningococcal disease provides epidemiologic evidence for the role of complement in host defense against this infection. Several reports have confirmed that deleting fHBP results in increased susceptibility of most strains of N. meningitidis to killing in either serum or in whole blood.

NMR and X-ray structures reveal that the protein is composed of two domains: an N-terminal domain of 8 beta-strands forming a highly curved anti-parallel beta-sheet (approximating a beta-barrel) and a C-terminal domain that is a well-defined beta-barrel of 8 anti-parallel beta strands. The 2 domains are connected by a short peptide, which together with several predominantly hydrophobic inter-domain contacts, results in minimal orientational flexibility between the two domains. Although there are over 300 different sequence variants of fHBP known, multiple-sequence alignments show that residues contributing to the hydrophobic cores of each domain are well conserved, suggesting that the 3D fold will be the same in all variants, even with sequence identity as low as 63% in some cases.

fHBP has a long N-terminal stretch containing a signal peptide and a lipo-box motif (LxxC). There are approximately 10 residues between the Cys and the folded N-terminal domain of fHBP and this linker region is highly flexible and has been removed from the protein in most structural studies. The structure of the lipidated form of the micelle-associated protein shows no significant differences when compared with the other known structures, suggesting that this N-terminal region serves simply to anchor the protein on the exterior bacterial surface, thus exposing fHBP to the solution and making it accessible to the host immune system.

The fHBP protein can be classified into three genetic and immunogenic variants: fHBP-1, fHBP-2 and fHBP-3, which are not cross-protective, and can be further divided into subvariants fHBP-1.x, fHBP-2.x and fHBP-3.x. Sequence conservation within each variant ranges from 85 to 100%, while between the variants the conservation can be as low as 63%. The level of similarity is 74.1% between variant 1 and variant2, 62.8% between variant 1 and variant3, and 84.7% between variant2 and variant3. Each variant has a substantial number of subvariants (more than 760 polypeptides) that have some associations with meningococcal CCs and scrogroups. In a different nomenclature scheme based on genetic information, the variants have been grouped into family A (variants 2-3) and family B (variant 1). Amino acid sequence identity within subfamily A is between 84.3% and 99.2%, whereas it ranges from 87.1% to 99.2% within subfamily B. The amino acid sequence of these two subfamilies shows an identity varying from 59.2% to 74.4%. A molecular subtyping analysis on 1837 strains from a worldwide collection showed that subfamily B sequences represent 70% of the isolates and subfamily A only 30%. Sequence variations were distributed throughout the length of the protein.

The level of fHBP can vary up to 15-fold among different clinical isolates and that the expression level is genetically determined, with expression of variant 1 generally higher compared to the expression of the other two variants (variant 2 and 3).

fHBP is able to induce bactericidal antibodies that confer passive protection in an infant rat model of bacteremia. The following variables can be measured to determine the efficacy of anti-fHBP antibodies in the killing of meningococcus: i) the ability of the antibody to block fH binding to the bacterial surface (antibodies that block fH binding show greater bactericidal activity); ii) subclass of the Ab (murine IgG3>IgG2a/b, human IgG3>IgG1>IgG2 [68]; iii) presence of antibodies directed against distinct fHBP epitopes (synergy is seen with combinations of anti-fHBP mAbs directed against non-competing epitopes) and iv) the amount of fHBP expressed on the bacterial surface (epitope density). Moreover, it has been demonstrated that the bactericidal response induced by fHBP is dependent on amino acidic sequence diversity; different fHBP sub-variants induce different levels of cross reactivity suggesting that the selected fHBP subvariant is important for the breath and magnitude of bactericidal response.

The term “antigenic fragment” refers to any fragment of a protein that generates an immune response (humoral or T cell response) to the protein in vivo. In embodiments, the antigenic fragment includes the anchor sequence of fHBP. In embodiments, the antigenic fragment lacks the anchor sequence of fHBP. The antigenic fragment may be a linear epitope, discontinuous epitope, or a conformation epitope (e.g., a folded domain). The antigenic fragment may preserve the secondary, tertiary, and/or quaternary structure of the full-length protein. In embodiments, the antigenic fragment comprises a neutralizing epitope. In such cases, the protein nanostructure may generate a neutralizing antibody response. Antigenic fragments may be designed computationally, such as by predicting the secondary structure and rationally removing N- or C-terminal unstructured regions or internally loops, or entire structural elements (alpha helices and/or beta sheets).

Illustrative fHBP sequences are provided in Table 2. In embodiments, the disclosure provides a protein nanostructure, where the nanostructure includes a first component and a second component; the first component includes a factor H binding protein (fHBP), optionally of Neisseria meningitidis or Neisseria gonorrhoeae or any Neisseria bacterium, and a first polypeptide comprising a first assembly domain, and the second component comprises a polypeptide comprising a second assembly domain, wherein the first assembly domain and the second assembly domain comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 1. In embodiments, the fHBP comprises a sequence that has at least 70%, at least 80%, at least 90%, at least 95%, at least 96%, at least 97%, at least 98%, at least 99%, or 100% identity to any polypeptide sequence in Table 2.

TABLE 2

| Description | Sequence | SEQ ID NO: |
|---|---|---|
| WP_260391870 | VAADIGAGLADALTAPLDHKDKGLKSLTLEDSISQNGTL | 54 |
| | TLSAQSAERTFKAGDKDNSLNTGKLKNDKISRFDFIRQI | |
| | EVDGQLITLESGEFQVYKQSHSALTALQTEQVQDSEHSG | |
| | KMVAKRQFRIGDIAGEHTSFDKLPKDVMATYRGTAFGSD | |
| | DAGGKLTYTIDFAAKQGYGKIEHLKSPELNVDLAAANIE | |
| | QDEKHHAVISGSVLYNQDEKGSYSLGIFGEKFQEVAGSA | |
| | EVKTANGIHHIGLAAKQ | |
| WP_219225651 | VAADIGTGLADALTAPLDHKDKGLQSLTLDQSVRKNEKL | 55 |
| | KLAAQGAEKTYGNGDSLNTGKLKNDKISRFDFIRQIEVD | |
| | GQLITLESGEFQVYKQSHSALTALQTEQEQDPEHSGKMV | |
| | AKRRFKIGDIAGEHTSFDKLPKDVMATYRGTAFGSDDAG | |
| | GKLTYTIDFAAKQGHGKIEHLKSPELNVELATAYIKPDE | |
| | KRHAVISGSVLYNQNEKGSYSLGIFGGQAQEVAGSAEVE | |
| | TANGIHHIGLAAKQ | |
| WP_101123807 | VAADIGTGLADALTAPLDYKDKGLQSLMLDQSVRKNEKL | 56 |
| | KLSAQGAEKTYGNGDSLNTGKLKNDKVSRFDFIRQIEVD | |
| | GQLITLESGEFQVYKQSHSALTALQTEQVQDSEDSGKMV | |
| | AKRQFRIGDIAGEHTSFDKLPKGGSATYRGTAFGSDDAG | |
| | GKLTYTIDFAAKQGHGKIEHLKSPELNVDLATVDIKPDE | |
| | KHHAVISGSVLYNQDEKGSYSLGIFGGKAQEVAGSAEVK | |
| | TANGIRHIGLAAKQ | |
| WP_096111629 | VAADIGAGLADALTAPLDHKDKSLQSLTLDQSVRKNEKL | 57 |
| | KLAAQGAEKTYGNGDSLNTGKLKNDKVSRFDFIRQIEVD | |
| | GQLITLESGEFQVYKQSHSALTALQTEQVQDSEHSGKMV | |
| | AKRQFRIGDIAGEHTSFDKLPEGGRATYRGTAFGSDDAS | |
| | GKLTYTIDFAAKQGHGKIEHLKSPELNVDLAASDIKPDK | |
| | KRHAVISGSVLYNQAEKGSYSLGIFGGQAQEVAGSAEVE | |
| | TANGIRHIGLAAKQ | |
| WP_079888201 | VAADIGAGLADALTAPLDHKDKSLQSLTLDQSVRKNEKL | 58 |
| | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPSGKAEYHGKAFSFDDAGG | |
| | KLTYTIDFAAKQGHGKIEHLKTPEQNVELASAELKADEK | |
| | SHAVILGDTRYGGEEKGTYHLALFGDRAQEIAGSATVKI | |
| | REKVHEIGIAGKQ | |
| WP_139627162 | VAADIGAGLADALTAPLDHKDKGLQSLTLDQSVRKNEKL | 59 |
| | KLAAQGAEKTYGNGDSLNTGKLKNDKVSRFDFIRQIEVD | |
| | GKLITLESGEFQVYKQSHSALTALQTEQVQDSEDSGKMV | |
| | AKRQFRIGDIAGEHTSFDKLPKGGSATYRGTAFGSDDAG | |
| | GKLTYTIDFAAKQGHGKIEHLKSPELNVELATAYIKPDE | |
| | KRHAVISGSVLYNQDEKGSYSLGIFGGQAQEVAGSAEVE | |
| | TANGIHHIGLAAKQ | |
| WP_192888031 | VAADIGTGLADALTTPLDHKDKGLKSLTLEDSIPQNGTL | 60 |
| | TLSAQGAEKTFKAGDKDNSLNTGKLKNDKISRFDFVQKI | |
| | EVDGQTITLASGEFQIYKQDHSAVVALQIEKINNPDKTD | |
| | SLINQRSFLVSGLGGEHTAFNQLPGGKAEYHGKAFSSDD | |
| | PNGRLHYTIDFTNKQGYGRIEHLKTPEQNVELASAELKA | |
| | DEKSHAVILGDTRYGSEEKGTYHLALFGDRAQEIAGSAT | |
| | VKIGEKVHEIGIAGKQ | |
| EJU68883 | VAADIGAGLADALTAPLDHKDKSLQSLTLDQSVRKNEKL | 61 |
| | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPDGKAEYHGKAFSSDDAGG | |
| | KLTYTIDFAAKQGHGKIEHLKSPELNVELATAYIKPDEK | |
| | RHAVISGSVLYNQDEKGSYSLGIFGGQAQEVAGSAEVET | |
| | ANGIHHIGLAAKQ | |
| MBJ1822574 | VAADIGTGLADALTAPLDHKDKGLKSLTLEDSIPQNGTL | 62 |
| | TLSAQGAEKTFKAGDKDNSLNTGKLKNDKISRFDFVQKI | |
| | EVDGQTITLASGEFQIYKQNHSAVVALQIEKINNPDKID | |
| | SLINQRSFLVSGLGGEHTAFNQLPVGKAEYHGKAFSSDD | |
| | AGGKLTYTIDFAAKQGHGKIEHLKSPELNVELATAYIKP | |
| | DEKHHAVISGSVLYNQDEKGSYSLGIFGGQAQEVAGSAE | |
| | VETANGIRHIGLAAKQ | |
| AGA84409 | VAADIGAGLADALTAPLDHKDKSLQSLTLDQSVRKNEKL | 63 |
| | KLAAQGAEKTYGNGDSLNTGKLKNDKVSRFDFIHQIEVD | |
| | GQLITLESGEFQVYKQSHSALTALQTEQEQDPEHSGKMV | |
| | AKRRFKIGDIAGEHTSFDKLPKDVMATYRGTAFGSDDAG | |
| | GKLTYTIDFAAKQGHGKIEHLKSPELNVELATAYIKPDE | |
| | KHHAVISGSVLYNQDEKGSYSPRYLWRKSPRSCRAARKW | |
| | KPQTAYTISALAAKQ | |
| AAF42204.1 | VAADIGAGLADALTAPLDHKDKGLQSLTLDQSVRKNEKL | 64 |
| | KLAAQGAEKTYGNGDSLNTGKLKNDKVSRFDFIRQIEVD | |
| | GQLITLESGEFQVYKQSHSALTAFQTEQIQDSEHSGKMV | |
| | AKRQFRIGDIAGEHTSFDKLPEGGRATYRGTAFGSDDAG | |
| | GKLTYTIDFAAKQGNGKIEHLKSPELNVDLAAADIKPDG | |
| | KRHAVISGSVLYNQAEKGSYSLGIFGGKAQEVAGSAEVK | |
| | TVNGIRHIGLAAKQ | |
| WP_164730143 | VAADIGAGLADALTAPLDHKDKSLQSLTLDQSVRKNEKL | 65 |
| | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPSGKAEYHGKAFSSDDAGG | |
| | KLTYTIDFAAKQGHGRIEHLKTPEQNVELASAELKADEK | |
| | SHAVILGDTRYGGEEKGTYHLALFGDRAQEIAGSATVKI | |
| | REKVHEIGIAGKQ | |
| WP_183432219 | VAADIGAGLADALTAPLDHKDKGLQSLTLDQSVRKNEKL | 66 |
| | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPGGKAEYHGKAFSSDDPNG | |
| | RLHYSIDFTKKQGYGRIEHLKTPEQNVELASAELKADEK | |
| | SHAVILGDTRYGSEEKGTYHLALFGDRAQEIAGSATVKI | |
| | REKVHEIGIAGKQ | |
| AAR84481.1 | VTADIGTGLADALTAPLDHKDKGLKSLTLEDSISQNGTL | 67 |
| (Peptide 55, B01 | TLSAQGAEKTYGNGDSLNTGKLKNDKVSRFDFIRQIEVD | |
| v1.55) | GQLITLESGEFQVYKQSHSALTALQTEQEQDPEHSEKMV | |
| | AKRRFRIGDIAGEHTSFDKLPKDVMATYRGTAFGSDDAG | |
| | GKLTYTIDFAAKQGHGKIEHLKSPELNVDLAVAYIKPDE | |
| | KHHAVISGSVLYNQDEKGSYSLGIFGEKAQEVAGSAEVE | |
| | TANGIHHIGLAAKQ | |
| WP_079871034 | VAADIGAGLADALTAPFDHKDKGLQSLTLDQSVRKNEKL | 68 |
| | KLAAQGAEEIYGNGDSLDTGKLKNDKVSRFDFIRQIEVD | |
| | GQLITLESGEFQVYKQSHSALTALQTEQVQDSEHSRKMV | |
| | AKRQFRIGDIAGEHTSFDKLPEGGRATYRGTALGSDDAG | |
| | GKLIYTIDFAAKQGHGKIEHLKSPELNVDLAAAYIKPDE | |
| | KHHAVISGSVLYNQAEKGSYSLGIFGGKAQEVAGSAEVK | |
| | TVNGIRHIGLAAKQ | |
| ACI46789 | VAADIGAGLADALTAPLDHKDKGLQSLTLDQSVRKNEKL | 69 |
| | KLAAQGAEKTYGNGDSLNTGKLKNDKVSRFDFIRQIEVD | |
| | GKLITLESGEFQVYKQSHSALTALQTEQVQDSEDSGKMV | |
| | AKRQFRIGDIAGEHTSFDKLPKGGSATYRGTAFGSDDAG | |
| | GKLTYTIDFAAKQGHGKIEHLKTPEQNVELASAELKADE | |
| | KSHAVILGDTRYGGEEKGTYHLALFGDRAQEIAGSATVK | |
| | IREKVHEIGIAGKQ | |
| AGA84313 | VAAEIGAGLDDALTAPLDHKDKSLRSLTLDQSILKNEKL | 70 |
| | KLAAHGAENPYGNGDRLYTGKLKNDKVSRFDFIRPIEVD | |
| | GKVITLESGEFQIYKQDHSAVVALQIEKISNPDKIDGLI | |
| | NQRSFLVSGLGGEHTAFNQLPSGKAEYHGKAFSSDHAGG | |
| | KLTYTIDFAAKQGHGKIEHLKTPEQNVELASAELKADEK | |
| | HHAVILGDTRYGGEEKGTYHLALFGDRAQEIAGRATVDK | |
| | GKGSRQIGIAGKQ | |
| WP_127224880 | VAADIGAGLADALTAPLDHKDKGLQSLTLDQSVRKNEKL | 71 |
| | KLAAQGAEKTYGNGDSLNTGKLKNDKVSRFDFIRQIEVD | |
| | GQLITLESGEFQVYKQSHSALTALQTEQEQDLEHSRKMV | |
| | AKRRFKIGDIAGEHTSFDKLPKDVMATYRGTAFGSDDAG | |
| | GKLTYTIDFAAKQGHGKIEHLKSPELNVDLAVAYIKPDE | |
| | KHHAVISGSVLYNQDEKGSYSLGIFGEKAQEVAGSAEVK | |
| | TANGIHHIGLAAKQ | |
| AGA84316 | VAADIGAGLADALTAPLDHKDKGLQSLTLDQSVRKNEKL | 72 |
| | KLAAQGAEKTYGNGDSLNTGKLKNDKVSRFDFIRQIEVD | |
| | GQLITLESGEFQVYKQDHSALTALQIEKIQNSDHSGKMI | |
| | AKRSFIVGGLGGEHTSFDQLPEGGREYHRGTAFRSNDAE | |
| | KRLTYTIYFATKDGNGKIDHLHSPELSVLAAATYIKTDS | |
| | QRHAFCASSVIYAKTKERTYSLFFRSAPTKEVARRPEIK | |
| | SIKCINYLGLANKK | |
| WP_124203369 | VAADIGAGLADALTAPLDHKDKGLQSLTLNQSVRKKEKL | 73 |
| | KLAAQGAEKTYGNGDSLSTGKLKNDKVSRFDFIRQIEVD | |
| | GQLITLESGEFQVYKQSHSALTALQTEQVQDSEHSGKMV | |
| | AKRRFRIGDIAGEHTSFDKLPEGGRATYRGTAFSSDDAG | |
| | GKLIYTIDFAAKQGHGKIEHLKTPEQNVELASAELKADE | |
| | KSHAVILGDTRYGSEEKGTYHLALFGDRAQEIAGSATVK | |
| | IREKVHEIGIAGKQ | |
| WP_014581564 | VAADIGAGLADALTAPLDHKDKGLKSLTLEDSISQNGTL | 74 |
| | TLSAQGAERTFKAGDKDNSLNTGKLKNDKISRFDFIRQI | |
| | EVDGQLITLESGEFQVYKQSHSALTALQTEQVQDSEHSG | |
| | KMVAKRQFRIGDIVGEHTSFGKLPKDVMATYRGTAFGSD | |
| | DAGGKLTYTIDFAAKQGHGKIEHLKSPELNVDLAAADIK | |
| | PDEKHHAVISGSVLYNQAEKGSYSLGIFGGQAQEVAGSA | |
| | EVETANGIRHIGLAAKQ | |
| DQ523568 | VAADIGAGLADALTAPLDHKDKSLQSLTLDQSVRKNEKL | 75 |
| | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPDGKAEYHGKAFSSDDAGG | |
| | KLTYTIDFAAKQGHGKIEHLKTPEQNVELAAAELKADEK | |
| | SHAVILGDTRYGSEEKGTYHLALFGDRAQEIAGSATVKI | |
| | GEKVHEIGIAGKQ | |
| WP_192888105 | VAADIGTGLADALTTPLDHKDKGLKSLTLEDSIPQNGTL | 76 |
| | TLSAQGAEKTFKAGDKDNSLNTGKLKNDKISRFDFVQKI | |
| | EVDGQTITLASGEFQIYKQDHSAVVALQIEKINNPDKID | |
| | SLINQRSFLVSGLGGEHTAFNQLPGGKAEYHGKAFSSDD | |
| | AGGKLTYTIDFAAKQGHGKIEHLKTPEQNVELAAAELKA | |
| | DEKSHAVILGDTRYGSEEKGTYHLALFGDRAQEIAGSAT | |
| | VKIGEKVHEIGIAGKQ | |
| WP_231397958 | VAADIGAGLADALTAPLDHKDKGLKSLTLEDSIPQNGTL | 77 |
| | TLSAQGAEKTFKAGGKDNSLNTGKLKNDKISRFDFVQKI | |
| | EVDGQTITLASGEFQIYKQDHSAVVALQIEKINNPDKTD | |
| | SLINQRSFLVSGLGGEHTAFNQLPGGKAEYHGKAFSSDD | |
| | PNGRLHYTIDFTNKQGYGRIEHLKTPEQNVELASAELKA | |
| | DEKSHAVILGDTRYGSEEKGTYHLALFGDRAQEIAGSAT | |
| | VKIGEKVHEIGIAGKQ | |
| DQ523569 | VAADIGTGLADALTAPLDHKDKGLKSLTLEDSIPQNGTL | 78 |
| | TLSAQGAEKTFKAGDKDNSLNTGKLKNDKISRFDFVQKI | |
| | EVDGQTITLASGEFQIYKQNHSAVVALQIEKINNPDKTD | |
| | SLINQRSFLVSGLGGEHTAFNQLPGGKAEYHGKAFSSDD | |
| | PNGRLHYSIDFTKKQGYGRIEHLKTLEQNVELAAAELKA | |
| | DEKSHAVILGDTRYGSEEKGTYHLALFGDRAQEIAGSAT | |
| | VKIGEKVHEIGIAGKQ | |
| CBY90122.1 | VAADIGAVLADALTAPLDHKDKSLQSLTLDQSVRKNEKL | 79 |
| (MenA) | KLAAQGAEKTYGNGDSLNTGKLKNDKVSRFDFIRQIEVD | |
| | GQLITLESGEFQVYKQSHSALTALQTEQVQDSEHSGKMV | |
| | AKRQFRIGDIAGEHTSFDKLPEGGRATYRGTAFGSDDAS | |
| | GKLTYTIDFAAKQGHGKIEHLKSPELNVDLAASDIKPDK | |
| | KRHAVISGSVLYNQAEKGSYSLGIFGGQAQEVAGSAEVE | |
| | TANGIRHIGLAAKQ | |
| MC58 | VAADIGAGLADALTAPLDHKDKGLQSLTLDQSVRKNEKL | 80 |
| (MenB) | KLAAQGAEKTYGNGDSLNTGKLKNDKVSRFDFIRQIEVD | |
| | GQLITLESGEFQVYKQSHSALTAFQTEQIQDSEHSGKMV | |
| | AKRQFRIGDIAGEHTSFDKLPEGGRATYRGTAFGSDDAG | |
| | GKLTYTIDFAAKQGNGKIEHLKSPELNVDLAAADIKPDG | |
| | KRHAVISGSVLYNQAEKGSYSLGIFGGKAQEVAGSAEVK | |
| | TVNGIRHIGLAAKQ | |
| CAM09659.1 | VAADIGAGLADALTAPLDHKDKSLQSLTLDQSVRKNEKL | 81 |
| (MenC) | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPSGKAEYHGKAFSSDDPNG | |
| | RLHYSIDFTKKQGYGRIEHLKTPEQNVELASAELKADEK | |
| | SHAVILGDTRYGGEEKGTYHLALFGDRAQEIAGSATVKI | |
| | REKVHEIGIAGKQ | |
| CP021517.1 | VAADIGAGLADALTTPLDHKDKSLQSLTLDQSVRKNEKL | 82 |
| (MenY) | KLAAQGAEKTYGNGDSLNTGKLKNDKVSRFDFIRQIEVD | |
| | GQTITLASGEFQIYKQNHSAVVALQIEKINNPDKIDSLI | |
| | NQRSFLVSGLGGEHTAFNQLPDGKAEYHGKAFSSDDPNG | |
| | RLHYSIDFTKKQGYGRIEHLKTPEQNVELASAELKADEK | |
| | SHAVILGDTRYGGEEKGTYHLALFGDRAQEIAGSATVKI | |
| | REKVHEIGIAGKQ | |
| MBJ1831079.1 | VAADIGTGLADALTAPLDHKDKGLKSLTLEDSISQNGTL | 83 |
| (A05/v3.45) | TLSAQGAEKTFKVGDKDNSLNTGKLKNDKISRFDFVQKI | |
| | EVDGQTITLASGEFQIYKQDHSAVVALQIEKINNPDKID | |
| | SLINQRSFLVSGLGGEHTAFNQLPSGKAEYHGKAFSSDD | |
| | AGGKLTYTIDFAAKQGHGKIEHLKTPEQNVELASAELKA | |
| | DEKSHAVILGDTRYGSEEKGTYHLALFGDRAQEIAGSAT | |
| | VKIREKVHEIGIAGKQ | |
| WP_196960013.1 | VAADIGTGLADALTAPLDHKDKGLQSLTLDQSVRKNEKL | 84 |
| (MenW135) | KLAAQGAEKTYGNGDSLNTGKLKNDKVSRFDFIRQIEVD | |
| | GQLITLESGEFQIYKQDHSAVVALQIEKINNPDKIDSLI | |
| | NQRSFLVSGLGGEHTAFNQLPSGKAEYHGKAFSSDDPNG | |
| | RLHYSIDFTKKQGYGRIEHLKTPEQNVELASAELKADEK | |
| | SHAVILGDTRYGGEEKGTYHLALFGDRAQEIAGSATVKI | |
| | REKVHEIGIAGKQ | |
| GenBank: | VAADIGAGLADALTAPLDHKDKGLKSLTLEDSISQNGTL | 85 |
| ACI46780.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFGKLPKDVMATYRGTAFGSD | |
| | DAGGKLTYTIDFAAKQGHGKIEHLKSPELNVDLAAADIK | |
| | PDEKHHAVISGSVLYNQAEKGSYSLGIFGGQAQEVAGSA | |
| | EVETANGIRHIGLAAKQ | |
| GenBank: | VAADIGAGLADALTAPLDHKDKGLKSLTLEDSISQNGTL | 86 |
| JN580510.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIAGEHTSFDKLPEGGRATYRGTAFGSD | |
| | DAGGKLTYTIDFAAKQGNGKIEHLKSPELNVDLAAADIK | |
| | PDGKRHAVISGSVLYNQAEKGSYSLGIFGGKAQEVAGSA | |
| | EVKTVNGIRHIGLAAKQ | |
| GenBank: | VAADIGAGLADALTAPLDHKDKGLKSLTLEDSISQNGTL | 87 |
| ACM44953.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFDKLPKDVMATYRGTAFGSD | |
| | DAGGKLTYTIDFAAKQGHGKIEHLKSPELNVDLAAADIK | |
| | PDEKHHAVISGSVLYNQAEKGSYSLGIFGGQAQEVAGSA | |
| | EVETANGIRHIGLAAKQ | |
| GenBank: | VAADIGAGLADALTAPLDHKDKGLKSLTLEDSISQNGTL | 88 |
| ACI468.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFDKLPKDVMATYRGTAFGSD | |
| | DAGGKLTYTIDFAAKQGHGKIEHLKSPELNVDLAAADIK | |
| | PDEKHHAVISGSVLYNQAEKGSYSLGIFGGQAQEVAGSA | |
| | EVKTVNGIRHIGLAAKQ | |
| GenBank: | VAADIGAGLADALTAPLDHKDKGLKSLTLEDSISQNGTL | 89 |
| JN580511.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFDKLPKDVMATYRGTAFGSD | |
| | DAGGKLTYTIDFAAKQGHGKIEHLKSPELNVDLAAAYIK | |
| | PDEKHHAVISGSVLYNQDEKGSYSLGIFGEKAQEVAGSA | |
| | EVKTANGIHHIGLAAKQ | |
| GenBank: | VAADIGAGLADALTAPLDHKDKGLKSLTLEDSISQNGTL | 90 |
| ABL14232.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFGKLPKDVMATYRGTAFGSD | |
| | DAGGKLTYTIDFAAKQGHGKIEHLKSPELNVDLAAADIK | |
| | PDEKHHAVISGSVLYNQAEKGSYSLGIFGGQAQEVAGSA | |
| | EVETANGIRHIGLAAKQ | |
| GenBank: | VAADIGAGLADALTAPLDHKDKGLKSLTLEDSISQNGTL | 91 |
| ACB38149.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFGKLPKDVMATYRGTAFGSD | |
| | DAGGKLTYTIDFAAKQGHGKIEHLKSPELNVDLAAADIK | |
| | PDEKRHAVISGSVLYNQAEKGSYSLGIFGGKAQEVAGSA | |
| | EVKTVNGIRHIGLAAKQ | |
| GenBank: | VAADIGAGLADALTAPLDHKDKGLKSLTLEDSISQNGTL | 92 |
| ACM44948.1 | TLSAQGAERTFKAGDKDNSLNTGKLKNDKISRFDFIRQI | |
| (MenB) | EVDGQLITLESGEFQVYKQSHSALTALQTEQVQDSEHSG | |
| | KMVAKRQFRIGDIVGEHTSFGKLPKDVMATYRGTAFGSD | |
| | DAGGKLTYTIDFAAKQGHGKIEHLKSPELNVELATAYIK | |
| | PDEKHHAVISGSVLYNQDEKGSYSLGIFGGQAQEVAGSA | |
| | EVETANGIHHIGLAAKQ | |
| GenBank: | VAADIGAGLADALTAPLDHKDKGLQSLTLDQSVRKNEKL | 93 |
| ACI46929.1 | KLAAQGAEKTYGNGDSLNTGKLKNDKISRFDFIRQIEVD | |
| (MenB) | GQLITLESGEFQVYKQSHSALTALQTEQVQDSEDSGKMV | |
| | AKRQFRIGDIAGEHTSFDKLPEDVRATYRGTAFGSDDAG | |
| | GKLIYTIDFAAKQGHGKIEHLKSPELNVDLAAADIKPDE | |
| | KRHAVISGSVLYNQDEKGSYSLGIFGGKAQEVAGSAEVE | |
| | TANGIQHIGLAAKQ | |
| GenBank: | AADIGTGLADALTAPLDHKDKGLKSLTLEDSIPQNGTLT | 94 |
| ACJ04735 | LSAQGAEKTFKAGDKDNSLNTGKLKNDKISRFDFVQKIE | |
| (MenB) | VDGQTITLASGEFQIYKQDHSAVVALQIEKINNPDKIDS | |
| | LINQRSFLVSGLGGEHTAFNQLPGGKAEYHGKAFSSDDP | |
| | NGRLHYSIDFTKKQGYGRIEHLKTPEQNVELASAELKAD | |
| | EKSHAVILGDTRYGSEEKGTYHLALFGDRAQEIAGSATV | |
| | KIREKVHEIGIAGKQ | |
| GenBank: | VAADIGTGLADALTTPLDHKDKGLKSLTLEDSIPQNGTL | 95 |
| ACM44944 | TLSAQGAEKTFKAGDKDNSLNTGKLKNDKISRFDFVQKI | |
| (MenB) | EVDGQTITLASGEFQIYKQDHSAVVALQIEKINNPDKID | |
| | SLINQRSFLVSGLGGEHTAFNQLPSGKAEYHGKAFSSDD | |
| | AGGKLTYTIDFAAKQGHGKIEHLKTPEQNVELASAELKA | |
| | DEKSHAVILGDTRYGSEEKGTYHLALFGDRAQEIAGSAT | |
| | VKIREKVHEIGIAGKQ | |
| GenBank: | VAADIGTGLADALTAPLDHKDKGLKSLTLEDSIPQNGTL | 96 |
| GQ219769 | TLSAQGAEKTFKAGDKDNSLNTGKLKNDKISRFDFVQKI | |
| (MenB) | EVDGQTITLASGEFQIYKQDHSAVVALQIEKINNPDKID | |
| | SLINQRSFLVSGLGGEHTAFNQLPGGKAEYHGKAFSSDD | |
| | PNGRLHYSIDFTKKQGYGRIEHLKTPEQNVELASAELKA | |
| | DEKSHAVILGDTRYGSEEKGTYHLALFGDRAQEIAGSAT | |
| | VKIGEKVHEIGIAGKQ | |
| GenBank: | VAADIGTGLADALTAPLDHKDKGLKSLTLEDSIPQNGTL | 97 |
| ACI46934 | TLSAQGAEKTFKAGDKDNSLNTGKLKNDKISRFDFVQKI | |
| (MenB) | EVDGQTITLASGEFQIYKQDHSAVVALQIEKINNPDKID | |
| | SLINQRSFLVSGLGGEHTAFNQLPGGKAEYHGKAFSSDD | |
| | PNGRLHYSIDFTKKQGYGRIEHLKTPEQNVELASAELKA | |
| | DEKSHAVILGDTRYGSEEKGTYHLALFGDRAQEIAGSAT | |
| | VKIGEKVHEIGIAGKQ | |
| GenBank: | VAADIGAGLADALTAPLDHKDKGLQSLTLDQSVRKNEKL | 98 |
| ACI46837 | KLAAQGAEETYGNGDSLNTGKLKNDKVSRFDFIRQIEVD | |
| (MenB) | GQLITLENGEFQVYKQSHSALTALQTEQVQDSEHSGKMV | |
| | AKRQFRIGDIAGEHTSFDKLPEGGRATYRGTAFGSDDTG | |
| | GKLTYTIDFAAKQGHGKIEHLKSPELNVELATAYIKPDE | |
| | KRHAVISGSVLYNQDEKGSYSLGIFGGQAQEVAGSAEVE | |
| | TANGIQHIGLAAKQ | |
| N. gonorrhoeae | GTIKAGVETSRSVTHYGAQANRVKTATEIADLGSKIGFK | 99 |
| ABQ12655.1 | GQEDLGNGLKAIWQLEQKAYVSGTDTGWGNRQSFIGLKG | |
| (PorB.1a) | GFGKVRVGRLNNILKDTGGFNPWEGKSYYLGLSNIAQPE | |
| | ERPISVRYDSPEFAGFSGSVQYVPNDNSSKNHSESYHAG | |
| | FNYKNSGFFVQYAGSYKRHSYTTEKHQVHRLVGGYDHDA | |
| | LYASVAVQQQDAKLAWPDDNSHNSQTEVAATAAYRFGNV | |
| | TPRVSYAHGFKGSVYDADNDNTYDQVVVGAEY | |
| N. gonorrhoeae | GAIKAGVETSRSVAYHGAQADRVKTTTEIADLGSKIGFK | 100 |
| ABQ12647.1 | GQEDLGNGLKAIWQLEQKAYVSGTKEGWGSRQSFIGLKG | |
| (PorB.1a) | GFGKVRVGHLNNILKDTDGFNPWEGKSYYLGLSNIAQPE | |
| | ERHVSVRYDSPEFAGFSGSVQYVPNDNSGKNRSESYHAG | |
| | FNYKNSGFFVQYAGFYKRHNYTTEKHQVHRLVGGYDHDA | |
| | LYASVAVQQQDAKLTWSNDNSHNSQTEVAATAAYRFGNV | |
| | TPRVSYAHGFKGSVYDADNDNTYDQVVVGAEY | |
| N. gonorrhoeae | GTIKAGVETSRSVAYHGAQADRVKTTTEIADLGSKIGFK | 101 |
| ACD93459.1 | GQEDLGNGLKAIWQLEQKAYVSGTKEGWGSRQSFIGLKG | |
| (PorB.1a) | GFGKVRVGHLNNILKDTDGFNPWEGKSYYLGLSNIAQPE | |
| | ERHVSVRYDSPEFAGFSGSVQYVPNDNSGKNRSESYHAG | |
| | FNYKNSGFFVQYAGSYKRHNYTTEKHQVHRLVGGYDHDA | |
| | LYASVAVQQQDAKLTWSNDNSHNSQTEVAATAAYRFGNV | |
| | TPRVSYAHGFKGSVYDADND | |
| N. gonorrhoeae | WGNRQSFIGLKGGFGKVRVGRLNSVLKDTDGENPWEGKS | 102 |
| QEQ91611.1 | YYLGLSNIAQPEERHVSVRYDSPEFAGFSGSVQYVPNDN | |
| (PorB.1a) | SGKNHSESYHAGFNYKNSGFFVQYAGFYKRHNYTTEKHQ | |
| | VHRLVGGYDHDALYASVAVQQQDAKLTWRNDNSHNSQTE | |
| | VATTVAYRFGNVTPR | |
| N. gonorrhoeae | GTIKAGVETSRSVAHHGAQADRVKTATEIADLGSKIGFK | 103 |
| ABQ12645.1 | GQEDLGNGLKAIWQLEQKAYVSGTDTGWGNRQSFIGLKG | |
| (PorB.1a) | GFGKVRVGRLNNILKDTDGFNPWEGKNYYLGLSNIAQPE | |
| | ERHVSVRYDSPEFAGFSGSVQYVPNDNSGKNHSESYHAG | |
| | FNYKNSGFFVQYAGSYKRHNFTTEKHQVHRLVGGYDHDA | |
| | LYASVAVQQQDAKLAWSDDNSHNSQTEVAATAAYRFGNV | |
| | TPRVSYAHGFKGSVYDADNDNTYDQVVVGAEY | |

In embodiments, the fHBP includes a mutation that reduces binding to fH (“fH-nonbinding mutation”). In some embodiments, the fH-nonbinding mutation results in increased immunogencity to fHBP. In embodiments, the fHBP includes a stabilizing mutation. Nonlimiting examples include substitution of arginine 41 to serine in variant 1 (R41S) (Beernink P T, et al., J Immunol. 2011 Mar. 15; 186 (6): 3606-14), R41A in variant 1 (see Pajon R, et al., Infect Immun. 2012 August; 80 (8): 2667-77), R80A, D211A, E218A, E248A, T220A/H222A, and G236I in variant 2 (Id.), L130R/G133D, V131D. F129S. K219N, G220S, L130R/G133D/K219N, and L130R/G133D/G220S in variant 2 (Rossi R, et al., Infect Immun. 2016 May 24; 84 (6): 1735-1742), E283A, E304T and E283A/E304T in variant 1 (Johnson S, et al., PLOS Pathog. 2012; 8 (10): e1002981), T221A or D211A in variant 2 (Rossi R, et al., Vaccine. 2013 Nov. 4; 31 (46): 5451-7), Q38R, E92K, R130G, S223R (Konar M, et al., PLOS One. 2015 Jun. 9; 10 (6): e0128185), H248L in variant 1, T286A or E313A in variant 3. The skilled artisan can determine fHBP mutations that result in reduced binding of fH without effecting protein folding by standard methods. Illustrative fHBP sequences are provided in US 2019/0225655 A1 and US 2022/0265805 A1, the contents of which are incorporated herein in their entireties.

In embodiments, the protein nanostructures disclosed herein can include one or more additional protective antigens. Non-limiting examples include NadA, NspA, PorB, and NHBA, variants or antigenic fragments thereof. For example, the protein nanostructure can include Neisserial surface protein A (NspA), variant or antigenic fragment thereof. NspA is an integral membrane protein, highly conserved among N. meningitidis strains, with high sequence similarity to opacity-associated proteins, adhesins facilitating colonization of the human nasopharynx and mediating adhesion to the host cells. NspA binds fH and, like fHBP, binds preferentially hfH. The fH binding activity of NspA could enhance bacterial survival in nasopharynx and in the blood and is particularly important in the absence of capsule and lipooligosaccharide (LOS) sialylation. The protein nanostructure can include porin B (PorB), variant or antigenic fragment thereof. PorB is one of the most abundant outer membrane proteins, also binds to hfH and plays a role in enhancing serum resistance. The protein nanostructure can include Neisserial heparin binding antigen (NHBA), variant or antigenic fragment thereof. NHBA is able to bind heparin through an arginine-rich domain, enhancing meningococcal survival in the blood. Heparin is known to interact with some complement factors, including fH and vitronectin. Therefore, binding of NHBA to heparin on the bacterial cell surface may result in the recruitment of additional complement factors, such as fH.

In embodiments, the encoded polypeptide includes a peptide region that is a signal peptide. Signal peptides are well known in the art. The signal peptide may be the native signal peptide or can be replaced with another signal peptide. Signal peptides function to prompt a cell to translocate the protein, usually to the cellular membrane. The core of the signal peptide often contains a long stretch of hydrophobic amino acids (about 5-16 residues long) that has a tendency to form a single alpha-helix and is also referred to as the “h-region”. In addition, many signal peptides begin with a short positively charged stretch of amino acids, which may help to enforce proper topology of the polypeptide during translocation by what is known as the positive-inside rule. Because of its close location to the N-terminus it is called the “n-region”. At the end of the signal peptide there is typically a stretch of amino acids that is recognized and cleaved by signal peptidase.

In embodiments, the polypeptide does not include a signal peptide.

Nonlimiting examples of signal peptides are provided below:

(SEQ ID NO: 171)
MELLILKANAITTILTAVTFCFASG
(SEQ ID NO: 172)
MSWKVMIIISLLITPQHGL
(SEQ ID NO: 173)
MKAILVVLLYTFTTANA
(SEQ ID NO: 174)
MPISILLIITTMIMASHC
(SEQ ID NO: 175)
MFVFLVLLPLVSSQC

In embodiments, the protein nanostructure comprises, as a trimeric assembly, a first component comprising first assembly domains and, as pentameric assembly, a second component comprising second assembly domains. The first assembly domain comprises a protein-protein interface that induces a region of the first component to self-associate to form trimeric building blocks. In protein nanostructures that have two or more components, each copy of the first assembly domain further comprises a surface-exposed interface that interacts with a complementary surface-exposed interface on the second assembly domain. Similarly stated, the second assembly domain is adapted to multimerize with first assembly domain. As described in King et al. (Nature 510, 103-108, 2014), Bale et al. (Science 353, 389-394, 2016), and patent publications WO2014124301 A1 and US20160122392 A1, the complementary protein-protein interface between a first assembly domain and a second assembly domain drives the assembly of multiple copies of the trimeric assembly domain and second assembly domain into a target protein nanostructure. In embodiments, each of the trimeric assembly domains of the protein nanostructure bears an antigenic protein, or antigenic fragment thereof, linked thereto (e.g., as a genetic fusion); these protein nanostructures display the proteins at full valency. In other embodiments, the protein nanostructures of the disclosure comprise one or more first assembly domains bearing antigens proteins, or antigenic fragments thereof (e.g., as genetic fusions) as well as one or more first assembly domains that do not bear antigenic proteins; these protein nanostructures display the proteins at partial valency. The first assembly domains can be any polypeptide sequence that forms a trimer and interacts with a second assembly domains to drive assembly to a target protein nanostructure. In embodiments, the protein nanostructure comprises a first polypeptide and a second polypeptide selected from those disclosed in US20130274441 A1, US 2015/0356240 A1, US 2016/0122392 A1, WO 2018/187325 A1, each of which is incorporated by reference herein in its entirety.

In embodiments of the protein nanostructures of the present disclosure, the antigenic protein and the core of the protein nanostructure may be genetically fused such that they are both present in a single polypeptide, termed a “fusion protein.” The linkage between the protein and the core allows the antigenic protein, or antigenic fragment thereof, to be displayed on the exterior of the protein nanostructure. As such, the point of connection to the core can be on the exterior of the core of the protein nanostructure formed. A wide variety of polypeptide sequences can be used to link the proteins, or antigenic fragments thereof and the core of the protein nanostructure. In some cases the linker comprises a polypeptide sequence that can be included in the encoding polynucleotide sequence. Any suitable linker polypeptide can be used. In embodiments, the linker imposes a rigid relative orientation of the antigenic protein (e.g. fHBP) or antigenic fragment thereof to the core. In embodiments, the linker flexibly links the antigenic protein (e.g. fHBP) or antigenic fragment thereof to the core.

In embodiments, the encoded polypeptides can include a linker between regions. A wide variety of polypeptide sequences can be used and are well known in the art. In embodiments, the linker may comprise a Gly-Ser linker (i.e. a linker consisting of glycine and serine residues) of any suitable length. In embodiments, the Gly-Ser linker may be 3, 4, 5, 6, 7, 8, 9, 10, 11, 12, 13, 14, 15, 16, 17, 18, 19, 20 or more amino acids in length. Non-limiting examples of Glys-Ser linkers are presented below:

Illustrative polypeptide linkers are provided in Table 3. In embodiments, the polypeptide linker is any sequence in Table 3. In embodiments, the polypeptide linker comprises between 6 and 18 amino acids; about 8, about 12, or about 16 amino acids; or 8, 12, or 16 amino acids.

TABLE 3

| SEQ ID NO: | Linker |
|---|---|
| 145 | GGSGGSGS |
| 146 | GGSGGSGGSGGS |
| 147 | GGSGGSGSGGSGGSGS |
| 148 | GGSGGSGSGGSGGSGSGGSGSGGS |
| 149 | GGGGSGGGGSGGGGSGG |
| 150 | GGGGGSGGGSGGGGS |
| 151 | GGGGSGGGGSGGGGS |
| 152 | GGSGEKP |
| 153 | GGSGQKP |
| 154 | GGSGGSGEKP |
| 155 | GGSGGSGQKP |
| 156 | GSS |
| 157 | GSGS |
| 158 | GGSGGSGS |
| 159 | GSGGSGSGSGGS |
| 160 | GGSGGSGGSGGS |
| 161 | GGGGSGGGGSGGGGS |
| 162 | GGGGGSGGGSGGGGS |
| 163 | GGSGGSGSGGSGGSGS |
| 164 | GGGGSGGGGSGGGGSGG |
| 165 | GGSGGSGSGGSGGSGSGGSGSGGS |
| 166 | GGSGEKP |
| 167 | GGSGQKP |
| 168 | GGSGGSGEKP |
| 169 | GGSGGSGQKP |
| 170 | KSDELLGSGGSGSGSGGSEKAAKAEEAARK |

In embodiments of the protein nanostructures of the present disclosure, the first component may optionally contain a poly-His tag, HHHHHH (SEQ ID NO: 143).

Illustrative fusion proteins are provided in Table 4. In embodiments, the fusion protein comprises a polypeptide sequence at least 85%, at least 90%, at least 95%, at least 96%, at least 97%, at least 98%, at least 99%, or 100% identical to any sequence in Table 4.

TABLE 4

| SEQ | | | |
|---|---|---|---|
| ID NO: | fHBP Polypeptide Sequence | Notes 1 | Notes 2 |
| 104 | MGSSGGGGSGGGGVTADIGTGLADALTAPLDHKDKGLKSLTLE | fHBP with 8 | BOLD = fHBP |
| | DSISQNGTLTLSAQGAEKTYGNGDSLNTGKLKNDKVSRFDFIR | residue linker | seq |
| | QIEVDGQLITLESGEFQVYKQSHSALTALQTEQEQDPEHSEKM | | Ital. = GS linker |
| | VAKRRFRIGDIAGEHTSFDKLPKDVMATYRGTAFGSDDAGGKL | | Black = 153- |
| | TYTIDFAAKQGHGKIEHLKSPELNVDLAVAYIKPDEKHHAVIS | | 50A and His6 |
| | GSVLYNQDEKGSYSLGIFGEKAQEVAGSAEVETANGIHHIGLA | | tag |
| | AKQGGSGGSGSEKAAKAEEAARKMEELFKKHKIVAVLRANSVE | | |
| | EAIEKAVAVFAGGVHLIEITFTVPDADTVIKALSVLKEKGAII | | |
| | GAGTVTSVEQARKAVESGAEFIVSPHLDEEISQFAKEKGVFYM | | |
| | PGVMTPTELVKAMKLGHTILKLFPGEVVGPQFVKAMKGPFPNV | | |
| | KFVPTGGVNLDNVAEWFKAGVLAVGVGSALVKGTPDEVREKAK | | |
| | AFVEKIRGATELEHHHHHH | | |
| 105 | MGSSGGGGSGGGGVTADIGTGLADALTAPLDHKDKGLKSLTLE | fHBP with 12 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGGSGGSEKAAKAEEAARKMEELFKKHKIVAVLRANSV | | |
| | EEAIEKAVAVFAGGVHLIEITFTVPDADTVIKALSVLKEKGAIIGAGTVTS | | |
| | VEQARKAVESGAEFIVSPHLDEEISQFAKEKGVFYMPGVMTPTELVKA | | |
| | MKLGHTILKLFPGEVVGPQFVKAMKGPFPNVKFVPTGGVNLDNVAE | | |
| | WFKAGVLAVGVGSALVKGTPDEVREKAKAFVEKIRGATELEHHHHHH | | |
| 106 | MGSSGGGGSGGGGVTADIGTGLADALTAPLDHKDKGLKSLTLE | fHBP with 16 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGSGGSGGSGSEKAAKAEEAARKMEELFKKHKIVAVLR | | |
| | ANSVEEAIEKAVAVFAGGVHLIEITFTVPDADTVIKALSVLKEKGAIIGA | | |
| | GTVTSVEQARKAVESGAEFIVSPHLDEEISQFAKEKGVFYMPGVMTPT | | |
| | ELVKAMKLGHTILKLFPGEVVGPQFVKAMKGPFPNVKFVPTGGVNLD | | |
| | NVAEWFKAGVLAVGVGSALVKGTPDEVREKAKAFVEKIRGATELEHH | | |
| | HHHH | | |
| 107 | MGSSGGGGSGGGGVTADIGTGLADALTAPLDHKDKGLKSLTLE | fHBP with 24 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGSGGSGGSGSGGSGSGGSEKAAKAEEAARKMEELF | | |
| | KKHKIVAVLRANSVEEAIEKAVAVFAGGVHLIEITFTVPDADTVIKALSV | | |
| | LKEKGAIIGAGTVTSVEQARKAVESGAEFIVSPHLDEEISQFAKEKGVFY | | |
| | MPGVMTPTELVKAMKLGHTILKLFPGEVVGPQFVKAMKGPFPNVKF | | |
| | VPTGGVNLDNVAEWFKAGVLAVGVGSALVKGTPDEVREKAKAFVEKI | | |
| | RGATELEHHHHHH | | |
| 108 | MGSSGGGGSGGGGVTADIGTGLADALTAPLDHKDKGLKSLTLE | fHBP with 8 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGSEKAAKAEEAARKMEELFKKHKIVAVLRANSVEEAIE | | |
| | KAVAVFAGGVHLIEITFTVPDADTVIKALSVLKEKGAIIGAGTVTSVEQA | | |
| | RKAVESGAEFIVSPHLDEEISQFAKEKGVFYMPGVMTPTELVKAMKLG | | |
| | HTILKLFPGEVVGPQFVKAMKGPFPNVKFVPTGGVNLDNVAEWFKA | | |
| | GVLAVGVGSALVKGTPDEVREKAKAFVEKIRGATE | | |
| 109 | MGSSGGGGSGGGGVTADIGTGLADALTAPLDHKDKGLKSLTLE | fHBP with 12 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGGSGGSEKAAKAEEAARKMEELFKKHKIVAVLRANSV | | |
| | EEAIEKAVAVFAGGVHLIEITFTVPDADTVIKALSVLKEKGAIIGAGTVTS | | |
| | VEQARKAVESGAEFIVSPHLDEEISQFAKEKGVFYMPGVMTPTELVKA | | |
| | MKLGHTILKLFPGEVVGPQFVKAMKGPFPNVKFVPTGGVNLDNVAE | | |
| | WFKAGVLAVGVGSALVKGTPDEVREKAKAFVEKIRGATE | | |
| 110 | MGSSGGGGSGGGGVTADIGTGLADALTAPLDHKDKGLKSLTLE | fHBPwith 16 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGSGGSGGSGSEKAAKAEEAARKMEELFKKHKIVAVLR | | |
| | ANSVEEAIEKAVAVFAGGVHLIEITFTVPDADTVIKALSVLKEKGAIIGA | | |
| | GTVTSVEQARKAVESGAEFIVSPHLDEEISQFAKEKGVFYMPGVMTPT | | |
| | ELVKAMKLGHTILKLFPGEVVGPQFVKAMKGPFPNVKFVPTGGVNLD | | |
| | NVAEWFKAGVLAVGVGSALVKGTPDEVREKAKAFVEKIRGATE | | |
| 111 | MGSSGGGGSGGGGVTADIGTGLADALTAPLDHKDKGLKSLTLE | fHBPwith 24 | |
| | DSISQNGTLTLSAQGAEKTYGNGDSLNTGKLKNDKVSRFDFIR | residue linker | |
| | QIEVDGQLITLESGEFQVYKQSHSALTALQTEQEQDPEHSEKM | | |
| | VAKRRFRIGDIAGEHTSFDKLPKDVMATYRGTAFGSDDAGGKL | | |
| | TYTIDFAAKQGHGKIEHLKSPELNVDLAVAYIKPDEKHHAVIS | | |
| | GSVLYNQDEKGSYSLGIFGEKAQEVAGSAEVETANGIHHIGLA | | |
| | AKQGGSGGSGSGGSGGSGSGGSGSGGSEKAAKAEEAARKMEELF | | |
| | KKHKIVAVLRANSVEEAIEKAVAVFAGGVHLIEITFTVPDADTVIKALSV | | |
| | LKEKGAIIGAGTVTSVEQARKAVESGAEFIVSPHLDEEISQFAKEKGVFY | | |
| | MPGVMTPTELVKAMKLGHTILKLFPGEVVGPQFVKAMKGPFPNVKF | | |
| | VPTGGVNLDNVAEWFKAGVLAVGVGSALVKGTPDEVREKAKAFVEKI | | |
| | RGATE | | |
| 112 | MGSSGGGGSGGGGVTADIGTGLADALTAPLDHKDKGLKSLTLEDSIS | Soluble fHBP | |
| | QNGTLTLSAQGAEKTYGNGDSLNTGKLKNDKVSRFDFIRQIEVDGQLI | with C-term | |
| | TLESGEFQVYKQSHSALTALQTEQEQDPEHSEKMVAKRRFRIGDIAGE | His6 tag | |
| | HTSFDKLPKDVMATYRGTAFGSDDAGGKLTYTIDFAAKQGHGKIEHL | | |
| | KSPELNVDLAVAYIKPDEKHHAVISGSVLYNQDEKGSYSLGIFGEKAQE | | |
| | VAGSAEVETANGIHHIGLAAKQGGLEHHHHHH | | |
| 113 | MGSSGGGGSGGGGVTADIGTGLADALTAPLDHKDKGLKSLTLEDSIS | CompA-fHBP | |
| | QNGTLTLSAQGAEKTYGNGDSLNTGKLKNDKVSRFDFIRQIEVDGQLI | with C-term | |
| | TLESGEFQVYKQSHSALTALQTEQEQDPEHSEKMVAKRRFRIGDIAGE | His6 tag | |
| | HTSFDKLPKDVMATYRGTAFGSDDAGGKLTYTIDFAAKQGHGKIEHL | | |
| | KSPELNVDLAVAYIKPDEKHHAVISGSVLYNQDEKGSYSLGIFGEKAQE | | |
| | VAGSAEVETANGIHHIGLAAKQGGSGGSGGSGGSEKAAKAEEAARK | | |
| | MEELFKKHKIVAVLRANSVEEAIEKAVAVFAGGVHLIEITFTVPDADTVI | | |
| | KALSVLKEKGAIIGAGTVTSVEQARKAVESGAEFIVSPHLDEEISQFAKE | | |
| | KGVFYMPGVMTPTELVKAMKLGHTILKLFPGEVVGPQFVKAMKGPF | | |
| | PNVKFVPTGGVNLDNVAEWFKAGVLAVGVGSALVKGTPDEVREKAK | | |
| | AFVEKIRGATELEHHHHHH | | |
| 114 | EKAAKAEEAARKMEELFKKHKIVAVLRANSVEEAIEKAVAVFAGGVHLI | I53-50A and | |
| | EITFTVPDADTVIKALSVLKEKGAIIGAGTVTSVEQARKAVESGAEFIVSP | His6 tag | |
| | HLDEEISQFAKEKGVFYMPGVMTPTELVKAMKLGHTILKLFPGEVVGP | | |
| | QFVKAMKGPFPNVKFVPTGGVNLDNVAEWFKAGVLAVGVGSALVK | | |
| | GTPDEVREKAKAFVEKIRGATELEHHHHHH | | |

In embodiments, a single component self-assembles into the protein nanostructure. In embodiments, one or more purified samples of first and second components for use in forming a protein nanostructure are mixed in an approximately equimolar molar ratio in aqueous conditions (e.g., an I53-50A/B isosahedral protein nanostructure). The first and second components (through the assembly domains) interact with one another to drive assembly of the target protein nanostructure. Successful assembly of the target protein nanostructure can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled protein nanostructure can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the antigenic protein in the protein nanostructure can be assessed by techniques commonly used to determine the identity of protein molecules in aqueous solutions, including but not limited to SDS-PAGE, mass spectrometry, protein sequencing, ELISA, surface plasmon resonance, biolayer interferometry, or amino acid analysis. The accessibility of the protein on the exterior of the protein nanostructure, as well as its conformation or antigenicity, can be assessed by techniques commonly used to detect the presence and conformation of an antigen, including but not limited to binding by monoclonal antibodies, conformation-specific monoclonal antibodies, surface plasmon resonance, biolayer interferometry, or antisera specific to the antigen.

In various embodiments, the protein nanostructures of the disclosure comprise two or more distinct first polypeptides bearing different antigenic proteins as genetic fusions; these protein nanostructures co-display multiple different proteins (e.g. different variants of fHBP) on the same protein nanostructure. These multi-antigen protein nanostructures are produced by performing in vitro assembly with mixtures of two or more antigens each comprising a assembly domain. The fraction of each antigen in the mixture determines the average valency of each antigenic protein in the resulting protein nanostructures. The presence and average valency of each antigen in a given sample can be assessed by quantitative analysis using the techniques described above for evaluating the presence of antigenic proteins in full-valency protein nanostructures.

In various embodiments, the protein nanostructures are between about 20 nanometers (nm) to about 40 nm in diameter, with interior lumens between about 15 nm to about 32 nm across and pore sizes in the protein shells between about 1 nm to about 14 nm in their longest dimensions.

In embodiments, the protein nanostructure has icosahedral symmetry. In such embodiment, the protein nanostructure may comprise 60 copies of a first component and 60 copies of a second component. In one such embodiment, the number of identical first polypeptides in each first assembly is different than the number of identical first polypeptides in each second assembly. For example, in embodiments, the protein nanostructure comprises twelve first assemblies and twenty second assemblies; in such embodiments, each first assembly may, for example, comprise five copies of the identical first component, and each second assembly may, for example, comprise three copies of the identical second component. In other embodiments, the protein nanostructure comprises twelve first assemblies and thirty second assemblies; in such an embodiment, each first assembly may, for example, comprise five copies of the identical first component, and each second assembly may, for example, comprise two copies of the identical second component. In further embodiments, the protein nanostructure comprises twenty first assemblies and thirty second assemblies; in this embodiment, each first assembly may, for example, comprise three copies of the identical first component, and each second assembly may, for example, comprise two copies of the identical second component. All of these embodiments are capable of forming protein-based protein nanostructures with regular icosahedral symmetry.

In various further embodiments, oligomeric states of the first and second assembly domains are as follows:
I53-34A: trimer+I53-34B: pentamer;
I53-40A: pentamer+I53-40B: trimer;
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer;
I52-32A: dimer+I52-32B: pentamer; and
I52-33A: pentamer+I52-33B: dimer

In embodiments, the second assembly domain of the second polypeptide comprises a sequence that has at least 95% identity to I53-50A or a variant thereof:

I53-50A
(SEQ ID NO: 144)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQCRKAVES |
| 81 | GAEFIVSPHL DEEISQFCKE KGVFYMPGVM TPTELVKAMK |
| 121 | LGHTILKLFP GEVVGPQFVK AMKGPFPNVK FVPTGGVNLD |
| 161 | NVCEWFKAGV LAVGVGSALV KGTPDEVREK AKFKVEKIRG |
| 201 | CTE |

In embodiments, the second assembly domain has at least 90%, 95%, 96%, 97%, 98%, 99%, or 100% sequence identity to SEQ ID NO: 7 or SEQ ID NO: 144, or an antigenic fragment thereof.

The I53-50A protein sequence has two intra-monomer disulfide bonds. In embodiments, the cysteine residues are mutated to residues that do not contain a thiol group (e.g. alanine or serine). Removal of the thiol group may promote correct protein folding while not impairing multimerization. In embodiments, the assembly domain of the first polypeptide comprises an amino acid substitution at one or more of positions 74, 98, 163, and 201 relative to SEQ ID NO: 144.

In embodiments, the assembly domain of the first polypeptide comprises an amino acid substitution of one or more of C74A, C98A, C163A, and C201A relative to SEQ ID NO: 132. In embodiments, the assembly domain of the first polypeptide comprises SEQ ID NO: 132 or a variant thereof.

I53-50A-Acys (disulfide-free form)

| | (SEQ ID NO: 132) |
|---|---|
| 1 | MEELFKKHKI VAVLRANSVE EAIEKAVAVF AGGVHLIEIT |
| 41 | FTVPDADTVI KALSVLKEKG AIIGAGTVTS VEQARKAVES |
| 81 | GAEFIVSPHL DEEISQFAKE KGVFYMPGVM TPTELVKAMK |
| 121 | LGHTILKLFP GEVVGPQFNK AMKGPFPNVK FVPTGGVNLD |
| 161 | NVAEWFKAGV LAVGVGSALV KGTPDEVREK AKAFVEKIRG |
| 201 | ATE |

In embodiments, the assembly domain has at least 90%, 95%, 96%, 97%, 98%, 99%, or 100% sequence identity to SEQ ID NO: 144 or SEQ ID NO: 132, or an antigenic fragment thereof, and comprises one, two, three or four amino acid substitutions selected from C74A, C98A, C163A, and C201A. Alternatively, the substitution may be of C to A, T, S, L, I, or any amino acid other than C.

### Nucleic Acids

In another aspect, the present disclosure provides isolated nucleic acids encoding an antigen, a first component, and/or a second component, of the present disclosure. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

Illustrative polynucleotide sequence encoding fHBP protein are provided in Table 5. In some embodiments, the polynucleotide comprises at least 85%, at least 90%, at least 95%, at least 99%, or 100% identical to any sequence in Table 5.

TABLE 5

| SEQ ID NO: | fHBP necleic acid sequence | Note |
|---|---|---|
| 115 | ATGGGATCTTCCGGTGGTGGCGGTTCTGGCGGCGGTGGCGTTACGGCGGACATT | Soluble fHBP with |
| | GGTACTGGTCTCGCGGACGCCCTCACCGCACCTCTCGACCACAAAGACAAAGGTCT | C-term His6 tag |
| | GAAATCTCTGACGCTGGAAGACTCTATCTCTCAGAACGGTACCCTCACGCTGTCTG | |
| | CTCAGGGTGCGGAAAAAACCTACGGTAACGGTGACTCTCTGAACACCGGTAAACT | |
| | GAAAAACGACAAAGTTTCTCGTTTTGACTTCATCCGTCAGATCGAAGTAGACGGTC | |
| | AGCTCATCACCCTCGAATCTGGTGAATTCCAGGTTTACAAACAGTCTCACTCTGCGC | |
| | TGACGGCCCTGCAGACCGAACAAGAACAGGACCCGGAACACTCCGAAAAAATGG | |
| | TTGCGAAACGTCGTTTCCGTATCGGTGATATCGCTGGCGAACACACCTCTTTTGAC | |
| | AAACTGCCGAAAGACGTTATGGCGACCTACCGTGGTACCGCGTTTGGTAGCGATG | |
| | ACGCGGGTGGTAAGCTGACGTACACCATCGACTTCGCGGCTAAACAGGGTCACGG | |
| | TAAAATCGAGCACCTCAAATCTCCGGAACTGAATGTTGACCTGGCCGTAGCTTACA | |
| | TCAAACCGGATGAGAAACATCACGCGGTTATCTCTGGCTCCGTGCTGTACAACCAG | |
| | GACGAAAAAGGTTCTTACAGCCTGGGTATCTTCGGTGAAAAAGCTCAGGAAGTGG | |
| | CAGGTAGCGCGGAAGTTGAAACCGCGAATGGTATCCACCACATCGGCCTGGCAGC | |
| | CAAGCAAGGCGGCCTCGAGCACCACCACCACCACCAC | |
| 116 | ATGGGATCTTCCGGTGGTGGCGGTTCTGGCGGCGGTGGCGTTACGGCGGACATT | CompA-fHBP with |
| | GGTACTGGTCTCGCGGACGCCCTCACCGCACCTCTCGACCACAAAGACAAAGGTCT | C-term His6 tag |
| | GAAATCTCTGACGCTGGAAGACTCTATCTCTCAGAACGGTACCCTCACGCTGTCTG | |
| | CTCAGGGTGCGGAAAAAACCTACGGTAACGGTGACTCTCTGAACACCGGTAAACT | |
| | GAAAAACGACAAAGTTTCTCGTTTTGACTTCATCCGTCAGATCGAAGTAGACGGTC | |
| | AGCTCATCACCCTCGAATCTGGTGAATTCCAGGTTTACAAACAGTCTCACTCTGCGC | |
| | TGACGGCCCTGCAGACCGAACAAGAACAGGACCCGGAACACTCCGAAAAAATGG | |
| | TTGCGAAACGTCGTTTCCGTATCGGTGATATCGCTGGCGAACACACCTCTTTTGAC | |
| | AAACTGCCGAAAGACGTTATGGCGACCTACCGTGGTACCGCGTTTGGTAGCGATG | |
| | ACGCGGGTGGTAAGCTGACGTACACCATCGACTTCGCGGCTAAACAGGGTCACGG | |
| | TAAAATCGAGCACCTCAAATCTCCGGAACTGAATGTTGACCTGGCCGTAGCTTACA | |
| | TCAAACCGGATGAGAAACATCACGCGGTTATCTCTGGCTCCGTGCTGTACAACCAG | |
| | GACGAAAAAGGTTCTTACAGCCTGGGTATCTTCGGTGAAAAAGCTCAGGAAGTGG | |
| | CAGGTAGCGCGGAAGTTGAAACCGCGAATGGTATCCACCACATCGGCCTGGCAGC | |
| | CAAGCAAGGCGGCTCTGGTGGTTCTGGCGGCAGCGGCGGTTCCGAGAAAGCAGC | |
| | GAAAGCTGAAGAAGCTGCACGTAAAATGGAAGAACTGTTCAAAAAACACAAAATC | |
| | GTTGCAGTTCTGCGTGCGAACTCTGTTGAAGAAGCGATCGAAAAAGCGGTTGCGG | |
| | TTTTCGCGGGTGGTGTCCACCTCATCGAAATCACCTTCACCGTGCCGGACGCAGAT | |
| | ACCGTTATCAAAGCGCTGTCTGTTCTGAAAGAAAAAGGTGCGATCATCGGTGCGG | |
| | GTACGGTTACCTCTGTAGAACAGGCGCGTAAGGCGGTTGAATCTGGCGCGGAATT | |
| | CATCGTTTCTCCGCACCTCGACGAGGAAATCTCTCAGTTTGCGAAAGAGAAAGGTG | |
| | TTTTCTACATGCCGGGTGTTATGACCCCGACGGAACTGGTGAAAGCGATGAAGCT | |
| | GGGTCACACCATCCTGAAACTGTTTCCGGGTGAAGTTGTTGGTCCGCAGTTCGTAA | |
| | AAGCCATGAAAGGTCCGTTCCCTAATGTTAAATTCGTTCCGACCGGCGGTGTCAAC | |
| | CTGGACAACGTAGCGGAATGGTTCAAAGCAGGTGTTCTCGCGGTTGGTGTTGGCT | |
| | CTGCGCTGGTTAAAGGTACCCCGGACGAAGTTCGTGAAAAGGCAAAAGCGTTCGT | |
| | TGAAAAAATCCGTGGTGCGACCGAACTCGAGCACCACCACCACCACCAC | |

In embodiments, the polynucleotide is any sequence in Table 5.

In a further aspect, the present disclosure provides recombinant expression vectors comprising the isolated nucleic acid of any embodiment or combination of embodiments of the disclosure operatively linked a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector can be replicable in the host organisms either as an episome or by integration into host chromosomal DNA. Suitable expression vectors and hosts are well known in the art. In embodiments, the expression vector comprises a plasmid. However, the disclosure is intended to include other expression vectors that serve equivalent functions, such as viral vectors.

In another aspect, the present disclosure provides host cells that have been transfected or transduced with the recombinant expression vectors disclosed herein, wherein the host cells can be either prokaryotic or eukaryotic. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, NY).

In another aspect, the disclosure provides a method of producing an antigen, component, or protein nanostructure according to the disclosure. In embodiments, the method comprises the steps of (a) culturing a host according to this aspect of the disclosure under conditions conducive to the expression of the polypeptide, and (b) optionally, recovering the expressed polypeptide.

In embodiments, the disclosure provides a method of manufacturing a vaccine, comprising culturing a host cell comprising a polynucleotide comprising a sequence encoding the antigen of the disclosure in a culture medium so that the host cell secretes the antigen into the culture media; optionally purifying the antigen from the culture media; mixing the antigen with a second component, wherein the second component multimerizes with the antigen to form a protein nanostructure; and optionally purifying the protein nanostructure.

In embodiments, the disclosure provides method of manufacturing a vaccine, comprising culturing a host cell comprising one or more polynucleotides comprising sequences encoding both components of the protein nanostructure of any one of disclosure so that the host cell secretes the first component and the second component into the culture media; and optionally purifying the protein nanostructure from the culture media.

Illustrative host cells in include E. coli cells, 293 and 293F cells, HEK293 cells, Sf9 cells, Chinese hamster ovary (CHO) cells and any other cell line used in the production of recombinant proteins.

In various embodiments, the first component expresses at about 0.5 mg/mL, about 1.0 mg/mL, about 1.5 mg/mL, about 2.5 mg/mL, about 5 mg/mL, about 10 mg/mL, about 25 mg/mL, about 50 mg/mL, about 75 mg/mL, about 100 mg/mL, or greater in a method of manufacturing according to the disclosure (e.g. E. coli). In various embodiments, the first component expresses at least 50%, at least 60%, at least 70%, at least 80%, at least 90%, or at least 95% of the expression level of an fHBP (optionally the same fHBP as in the protein nanostructure) in the same or similar expression system. In various embodiments, the first component expresses at least 105%, at least 110%, at least 115%, at least 120%, at least 125%, at least 150%, at least 175%, or at least 200% of the expression level of an fHBP (optionally the same fHBP as in the protein nanostructure) in the same or similar expression system.

### Vaccines and Administration

The disclosure also provides vaccines comprising the protein nanostructures described herein. Such compositions can be used to raise antibodies in a mammal (e.g. a human). The vaccines compositions of the disclosure typically include a pharmaceutically acceptable carrier, and a thorough discussion of such carriers is available in Remington: The Science and Practice of Pharmacy.

The pH of the composition is usually between about 4.5 to about 11, such as between about 5 to about 11, between about 5.5 to about 11, between about 6 to about 11, between about 5 to about 10.5, between about 5.5 to about 10.5, between about 6 to about 10.5, between about 5 to about 10, between about 5.5 to about 10, between about 6 to about 10, between about 5 to about 9.5, between about 5.5 to about 9.5, between about 6 to about 9.5, between about 5 to about 9, between about 5.5 to about 9, between about 6 to about 9, between about 5 to about 8.5, between about 5.5 to about 8.5, between about 6 to about 8.5, between about 5 to about 8, between about 5.5 to about 8, between about 6 to about 8, about 4.5, about 5, about 6.5, about 6, about 6.5, about 7, about 7.5, about 8, about 8.5, about 9, about 9.5, about 10, about 10.5, about 11, etc. Stable pH may be maintained by the use of a buffer e.g. a Tris buffer, a citrate buffer, phosphate buffer, or a histidine buffer. Thus a composition will generally include a buffer.

A composition may be sterile and/or pyrogen free. Compositions may be isotonic with respect to humans.

A vaccine composition comprises an immunologically effective amount of its antigen(s). An “immunologically effective amount” or “effective amount” is an amount which, when administered to a subject, is effective for eliciting an antibody response against the antigen. This amount can vary depending upon the health and physical condition of the individual to be treated, their age, the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the disclosure will generally be expressed in terms of the mass of protein per dose. A dose of 10-500 μg (e.g. 50 μg) per antigen can be useful.

Vaccine compositions may include an immunological adjuvant. Illustrative adjuvants include the following: 1. mineral-containing compositions; 2. oil emulsions; 3. saponin formulations; 4. virosomes and virus-like particles; 5. bacterial or microbial derivatives; 6. bioadhesives and mucoadhesives; 7. liposomes; 8. polyoxyethylene ether and polyoxyethylene ester formulations; 9. polyphosphazene (pcpp); 10. muramyl peptides; 11. imidazoquinolone compounds; 12. thiosemicarbazone compounds; 13. tryptanthrin compounds; 14. human immunomodulators; 15. lipopeptides; 16. benzonaphthyridines; 17. microparticles; 18. immunostimulatory polynucleotide (such as rna or dna; e.g., cpg-containing oligonucleotides).

For example, the composition may include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al⁺⁺⁺ in a composition for administration to a patient may be less than 5 mg/ml e.g. <4 mg/ml, <3 mg/ml, <2 mg/ml, <1 mg/ml, ctc. A preferred range is between 0.3 and 1 mg/ml. A maximum of 0.85 mg/dose is preferred. Aluminum hydroxide and aluminium phosphate adjuvants are suitable for use with the disclosure.

Exemplary adjuvants that may be used in a pharmaceutical composition provided herein include, but are not limited to, 3M-052, Adju-Phos™, Alhydrogel™, Adjumer™, albumin-heparin microparticles, Algal Glucan, Algammulin, Alum, Antigen Formulation, AS-2 adjuvant, ASO1, ASO3, autologous dendritic cells, autologous PBMC, Avridine™, B7-2, BAK, BAY R1005, BECC TLR-4 agonists, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CPG-1018, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/1g plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT (R192G), LT-OA or LT Oral Adjuvant, LT-R192G, LTK63, LTK72, Matrix-M™ adjuvant, MF59, MONTANIDE ISA 51, MONTANIDE ISA 720, MPL.™., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Plcuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rchydragel HPA, Rchydragel LV, RIBI, Ribi like adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, SWE, Tetanus toxoid (TT), Theramide™, Threonyl muramyl dipeptide (TMDP), Ty Particles, and Walter Reed Liposomes.

In preferred embodiments, the adjuvant is an aluminium hydroxide gel (e.g., Alhydrogel™). In preferred embodiments, the adjuvant is SWE. In preferred embodiments, the adjuvant is MF59.

MF59 is an oil-in-water emulsion containing squalene (4.3%) in citric acid buffer with stabilizing nonionic surfactants Tween 80 (0.5%) and Span 85 (0.5%). MF59 has been shown to be well-tolerated in humans and is used in vaccines against seasonal influenza (see Ko and Kang, Hum Vaccin Immunother. 2018; 14 (12): 3041-3045; U.S. Pat. No. 6,299,884).

For example, the composition may include an aluminum salt adjuvant, an oil in water emulsion (e.g. an oil-in-water emulsion comprising squalene, such as MF59, SWE, or AS03), a TLR9 agonist (such as CpG oligodeoxynucleotides), a TLR7 agonist (such as imidazoquinoline or imiquimod), or a combination thereof. In embodiments, the adjuvant is a combination of an aluminum salt and CPG1018. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al+++ in a composition for administration to a patient may be less than 5 mg/ml e.g. <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminium phosphate adjuvants are suitable for use with the disclosure. In a preferred embodiment, a pharmaceutical composition provided herein comprises aluminum hydroxide as an adjuvant. In some embodiment, a pharmaceutical composition provided herein comprises 500 μg aluminium hydroxide.

Selection of an adjuvant depends on the subject to be treated. Preferably, a pharmaceutically acceptable adjuvant is used.

In embodiments, the adjuvant is a squalene emulsion.

In embodiments, the adjuvant is a TLR4 immunostimulant (e.g., SLA, GLA), e.g., as described in Van Hoeven at al. PLOS One. 11 (2): c0149610 (2016).

In embodiments, the adjuvant is a TLR7/8 immunostimulant (e.g., R848, IMQ, 3M-052), e.g., as described in Dowling D. ImmunoHorizons (6): 185-197 (2018).

In embodiments, the adjuvant is a TLR9 immunostimulant (CpG), e.g., as described in Bode et al. Expert Rev Vaccines. 10 (4): 499-511 (2011).

In embodiments, the adjuvant is saponin (QS21), e.g., as described in Zhu et al. Nat Prod Chem Res. 3 (4): c113 (2016).

In embodiments, the vaccine comprises a combination of two or more adjuvants (e.g. squalene emulsion and alum or a TLR4 immunostimulant).

One suitable immunological adjuvant comprises a compound of Formula (I) as defined in WO2011/027222, or a pharmaceutically acceptable salt thereof, adsorbed to an aluminum salt. Many further adjuvants can be used, including any of those disclosed in Powell & Newman (1995).

Compositions may include an antimicrobial, particularly when packaged in multiple dose format. Antimicrobials such as thiomersal and 2-phenoxyethanol are commonly found in vaccines, but sometimes it may be desirable to use either a mercury-free preservative or no preservative at all.

Compositions may comprise detergent e.g. a polysorbate, such as polysorbate 80.

Detergents are generally present at low levels e.g. <0.01%.

Compositions may include sodium salts (e.g. sodium chloride) to give tonicity. A concentration of 10±2 mg/ml NaCl is typical e.g. about 9 mg/ml.

In embodiments, the buffer in the vaccine composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Illustrative tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the protein nanostructure, in lyophilized or liquid form. Illustrative stabilizers include sucrose, sorbitol, glycine, inositol, sodium chloride, methionine, arginine, and arginine hydrochloride.

In embodiments, the disclosure provides a vaccine (immunogenic composition) comprising one or more pharmaceutically acceptable excipients.

In embodiments, the vaccine (immunogenic composition) is a stable emulsion.

In embodiments, the disclosure provides a vaccine (immunogenic composition) comprises one or more adjuvants. In embodiments, the one or more adjuvants comprises a TLR4 immunostimulant, e.g., Monophosphoryl Lipid A (MPL), Glucopyranosyl Lipid A (GLA), and/or Soluble Leishmania Antigen (SLA).

In another aspect, the disclosure provides a method of inducing an immune response against fHBP, comprising administering to a subject in need thereof an immunologically effective amount of the immunogenic composition described herein, which comprises the protein nanostructure as described herein.

In certain embodiments, the immune response comprises the production of neutralizing antibodies against an infectious agent (e.g. Neisseria meningitidis). In certain embodiments, the neutralizing antibodies are complement-independent.

The immune response can comprise a humoral immune response, a cell-mediated immune response, or both. In embodiments an immune response is induced against each delivered antigenic protein. A cell-mediated immune response can comprise a Helper T-cell (Th) response, a CD8+ cytotoxic T-cell (CTL) response, or both. In embodiments the immune response comprises a humoral immune response, and the antibodies are neutralizing antibodies.

A useful measure of antibody potency against Neisseria meningitidis in the art is “bactericidal titer”. To determine 50% bactericidal titer, serum from immunized animals is diluted to assess how dilute serum can be yet attain 50% killing of the target bacteria (e.g. Neisseria meningitidis) in a serum bactericidal assay (SBA). For example, an SBA titer of 700 means that scrum attained the ability to kill 50% of bacteria after being diluted 700-fold. Thus, higher SBA titers indicate more potent neutralizing antibody responses. In embodiments, this titer is in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50% neutralization titer range can have an upper limit of about 400, about 600, about 800, about 1000, about 1500, about 2000, about 2500, about 3000, about 3500, about 4000, about 4500, about 5000, about 5500, about 6000, about 6500, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 25000.

Compositions of the disclosure will generally be administered directly to a subject. Direct delivery may be accomplished by parenteral injection (e.g. subcutaneously, intraperitoneally, intravenously, intramuscularly, or to the interstitial space of a tissue), orally, intranasal, or by any other suitable route. For example, intramuscular administration may be used e.g. to the thigh or the upper arm. Injection may be via a needle (e.g. a hypodermic needle), but needle-free injection may alternatively be used. A typical intramuscular dosage volume is 0.5 ml.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.). Multiple doses may be administered at least 1 month apart (e.g., about 2 months, about 3 months, about 4 months, about 6 months, about 8 months, about 10 months, about 12 months, about 16 months, etc.). A second or subsequent does may be administered over longer intervals, e.g., about 1 year or about 2 years after the previous dose.

Where the vaccine is for prophylactic use, the human is preferably a child (e.g., a toddler or infant), a teenager, or an adult; where the vaccine is for therapeutic use, the human is preferably a teenager or an adult. A vaccine intended for children may also be administered to adults, e.g., to assess safety, dosage, immunogenicity, etc.

Vaccines of the disclosure may be prophylactic (i.e. to prevent disease) or therapeutic (i.e. to reduce or eliminate the symptoms of a disease). The term prophylactic may be considered as reducing the severity of or preventing the onset of a particular condition. For the avoidance of doubt, the term prophylactic vaccine may also refer to vaccines that ameliorate the effects of a future infection, for example by reducing the severity or duration of such an infection.

Dosages may be varied depending upon the requirements of the subject and the compound being employed. The dose administered to a subject, in the context of the present disclosure, should be sufficient to effect a beneficial prophylactic therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Alternatively, two doses of the protein nanostructure may be administered at a predetermined interval to achieve a prime-boost effect. The predetermined interval may be 1, 2, 3, 4, 6, 7, 10, or 14 days; or 3-5 days, 7-10 days, or 10-14 days or the like. The predetermined interval may be 1, 2, 3, 4, or 6 weeks; or 2-3 weeks, 3-4 weeks, or 5-6 weeks or the like. The predetermined interval may be 1, 2, 3, or 4 months.

The disclosure further provides combination vaccines. The vaccines of the disclosure include vaccines comprising both an fHBP protein nanostructure and one or more vaccines to another microbial pathogen (e.g. bacterium or virus) or also to Neisseria meningitidis. Exemplary vaccines can be directed to: Corynebacterium diphtheria (diphtheria), Clostridium tetani (tetanus), Bordetella pertussis (pertussis or whooping cough) Haemophilus influenzae type b (meningitis), Streptococcus pneumonia (bloodstream infections, pneumonia and meningitis), Salmonella typhi (typhoid), Mycobacterium tuberculosis (tuberculosis), Yersinia pestis (bubonic plague), Bacillus anthracis (anthrax), and Vibrio cholera (cholera). Other exemplary vaccines can be directed to: SARS-CoV-2, respiratory syncytial virus, Rabies, Hepatitis A, Polio, Hepatitis B, Yellow Fever, Japanese encephalitis, Parvovirus, Distemper, Adenovirus, Parainfluenza, Influenza, Measles, Lyme disease, Coronavirus, Vesicular stomatitis virus, Herpes simplex virus, Baculovirus, Thogotovirus, and Bornaviridac.

Also provided herein are kits for administration of nucleic acid (e.g., RNA), purified proteins, and purified protein nanostructures described herein, and instructions for use. The disclosure also provides a delivery device pre-filled with a composition or a vaccine disclosed herein.

The pharmaceutical compositions described herein can be administered in combination with one or more additional therapeutic agents. The additional therapeutic agents may include, but are not limited to antibiotics or antibacterial agents, antiemetic agents, antifungal agents, anti-inflammatory agents, antiviral agents, immunomodulatory agents, cytokines, antidepressants, hormones, alkylating agents, antimetabolites, antitumour antibiotics, antimitotic agents, topoisomerase inhibitors, cytostatic agents, anti-invasion agents, antiangiogenic agents, inhibitors of growth factor function inhibitors of viral replication, viral enzyme inhibitors, anticancer agents, α-interferons, β-interferon, ribavirin, hormones, and other toll-like receptor modulators, immunoglobulins (Igs), and antibodies modulating Ig function (such as anti-IgE (omalizumab)).

In certain embodiments, the compositions disclosed herein may be used as a medicament, e.g., for use in inducing or enhancing an immune response in a subject in need thereof, such as a mammal.

In certain embodiments, the compositions disclosed herein may be used in the manufacture of a medicament for inducing or enhancing an immune response in a subject in need thereof, such as a mammal.

One way of checking efficacy of therapeutic treatment involves monitoring infection by an infectious agent after administration of the compositions or vaccines disclosed herein. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

### EXAMPLES

The following Examples are merely illustrative and are not intended to limit the scope or content of the invention in any way.

### Example 1: Materials and Methods

This example describes materials and methods used in the examples.

Expression of Soluble fHBP and CompA-fHBP in E. coli

The I53-50A multimerization domain was C-terminally fused to the B01 fHBP variant, peptide 55, by a linker of 12 residues and a His₆ tag for purification was also included at the C-terminus of I53-50A. Soluble C-terminal His₆ tagged fHBP antigens, peptide 45 (also known as A05) and peptide 55, were also generated as controls and for a binding assay. The three fHBP proteins (termed “component A” or “CompA”) were expressed in Lemo21(DE3) (NEB) in TB (terrific broth) grown in 1 L baffled shake flasks. Cells were incubated at 37° C. to an OD600 ˜0.8, then induced with 1 mM IPTG (isopropylthio-β-galactoside). The cells were harvested and lysed via sonication using a QSonica Sonicator in 20 mM Tris pH 8.0, 150 mM NaCl, 30 mM imidazole, 0.1 mg/mL lysozyme, 0.75% Zwittergent 3-14. Lysates were clarified by centrifugation at 20,000×g for 1 hour at 4° C. and sterilized using a 50 mL conical tube/funnel 0.2 μm filtration units. Clarified lysates were applied to Ni²⁺ Indigo resin for purification by IMAC. Proteins of interest were eluted with 20 mM Tris pH 8.0, 150 mM NaCl, 300 mM imidazole, 0.75% Zwittergent 3-14 and then dialyzed into 20 mM Tris pH 8.0, 150 mM NaCl, 5% glycerol. The fHBP-CompA was purified by SEC by applying sample to a Superdex 200 Increase 10/300 GLcolumn (Cytiva) on FPLC (AKTA Pure) using 20 mM Tris pH 8.0, 150 mM NaCl, 5% glycerol buffer. Recombinant peptides for binding assays were purified by a Superdex 200 Increase HiScale 26/40 column (Cytiva) on an FPLC (AKTA Avant) with 20 mM Tris pH 8.0, 150 mM NaCl, 5% glycerol buffer.

### SDS-PAGE

SDS-PAGE samples were produced by adding 10 μL of 2× Laemmli buffer (Bio Rad) containing 5% BME to PCR tubes and adding 10 μL of purified soluble fHBP, CompA-fHBP or VLP sample and buffer to normalize to a 1.5 μg load in the gel. Gel samples were boiled at 95° C. for 10 minutes then centrifuged briefly to collect condensation. Samples were mixed well before loading 10 μL of each expression sample or 5 μL of normalized samples and 3 μL of Precision Plus dual color size standard (Bio-Rad) to the Tris/Glycine 4-15% TGX precast gels (Bio Rad) in a Criterion Gel Electrophoresis Cell. The gels were run at 200 V for 35 minutes in 1× Tris/Glycine/SDS buffer (Bio Rad). The gel was stained using AquaStain Protein Stain (Bulldog Bio) and imaged using UVP ChemStudio Plus Gel Doc (Bio Rad).

### Differential Scanning Fluorimetry

Nano-DSF thermal ramp was used to estimate the Tonset and melting temperature (Tm) of CompA-fHBP using SYPRO Orange Protein Gel Stain (Invitrogen) on an UNcle Nano-DSF (UNchained Laboratories). SYPRO was diluted from 5000× to a 200× working stock solution by adding 4 μL of SYPRO to 96 μL of buffer. Then, 3.5 μL of the 200× stock solution was added to 31.5 μL of CompA-fHBP sample in a PCR tube to bring SYPRO to 20×. The CompA-fHBP dilution with SYPRO was applied to quartz capillary cassettes (UNi, UNchained Laboratories) in triplicate and placed in the UNcle. Data were collected using a temperature ramp from 15° C. to 95° C. (holding samples at 15° C. for 300 seconds prior to data collection), collecting data at 0.5° C. increments.

### Bio-Layer Interferometry (BLI) for JAR4, JAR5, and JAR41 Binding

Antibodies JAR4, JAR5, and JAR41 (EMD Millipore) were normalized in concentration to 10 μg/mL in BLI assay buffer (PBS, 0.5% BSA, 0.05% Tween 20, pH 7.4) in a sufficient volume to load 200 μL per well of a black 96-well plate (Greiner Bio-one, 655076). Briefly, on an Octet Red96 instrument, Protein G biosensors (Sartorius, 18-5082) were dipped into assay buffer for 60 seconds to achieve a baseline. Next, the biosensors were dipped into each antibody for 60 seconds in order to immobilize the antibodies present but without reaching saturation, followed by an additional baseline step. The immobilized antibodies were allowed to associate with 200 μL of 10 μg/mL fHBP soluble protein, CompA-fHBP or fHBP VLPs for 150 seconds, and then the biosensors were dipped back into assay buffer for 150 seconds to observe any possible dissociation.

### Assembly of VLPs and SEC Purification

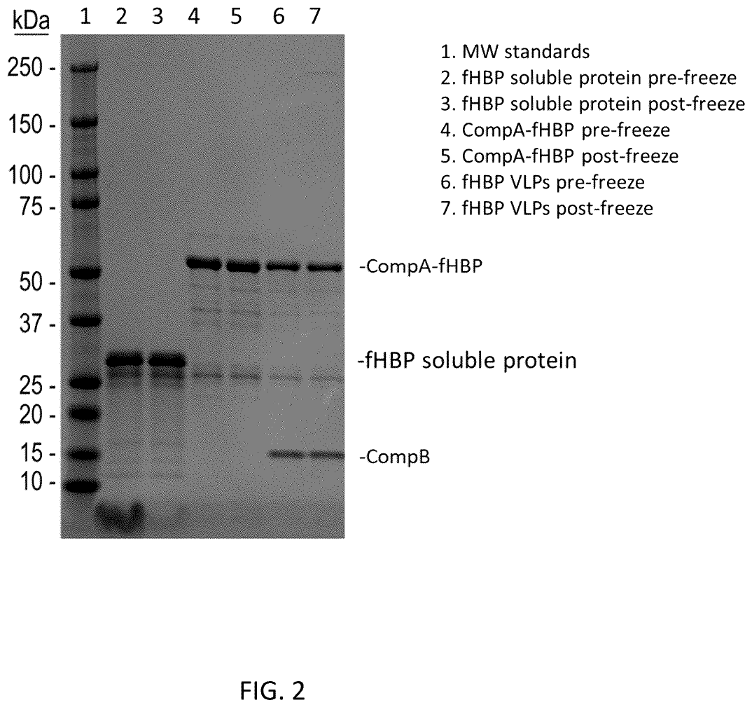

Molar concentrations for CompA-fHBP and CompB were determined using UV-Vis spectroscopy. Absorbance values at 280 nm were collected and divided by calculated molar extinction coefficients (ExPASy). The assembly reaction to produce fHBP antigen-bearing VLPs was performed in vitro with the addition of components as follows: CompA-fHBP was added to a tube to a final concentration of 14.35 μM, then assembly buffer (20 mM Tris, 150 mM NaCl, 5% glycerol, pH 8.0) as added to bring the final volume of the reaction to 2 mL, and finally CompB was added to the tube to a final concentration of 10.4 μM. The reaction was incubated at ambient temperature for ˜30 minutes with gentle rocking. The fHBP-VLP assembly was applied to a Superdex 6 Increase 10/300GLcolumn (Cytiva) on FPLC (AKTA Pure) using 20 mM Tris, 150 mM NaCl, 5% glycerol, pH 8.0 buffer. fHBP-VLP clutes at ˜10 mL and excess CompA-fHBP clutes at ˜15 mL. fHBP-VLP peak pooled (FIG. 2). fHBP displaying VLPs pre- and post-freeze/thaw cycle were analyzed on an SDS PAGE gel as described above.

### Dynamic Light Scattering (DLS)

DLS was used to measure hydrodynamic diameter (Dh) and polydispersity (% Pd) of the fHBP VLP assembly on an UNcle Nano-DSF (UNchained Laboratories). The set up included increased viscosity due to 5% glycerol in the buffer that was accounted for by the UNcle Client Software in Dh measurements. The fHBP VLP assembly was applied to quartz capillary cassettes (UNi, UNchained Laboratories) in triplicates and measured using the laser autoattenuation with 10 acquisitions per sample and 5 seconds per acquisition. Data were collected at 22° C. and the VLPs were monodisperse of the expected size.

Negative Stain Electron Microscopy (nsEM)

fHBP-VLP pre- and post-freeze samples were diluted to 75 μg/mL in 20 mM Tris, 150 mM NaCl, 5% glycerol, pH 8.0 and 3 μL of sample was applied to the carbon side of two glow-discharged (Pelco EasiGLOW) thick carbon copper 400 mesh grids (EMS, CF400-Cu-TH). Samples were incubated on the grids for ˜1 minute, then blotted away using grade 1 filter paper (Whatman). Immediately, 3 μL of 0.75% UF stain was applied to the carbon side of the grids and incubated for ˜1 minute. The stain was blotted away using filter paper and the application of stain and blotting was repeated 2 more times. The grids were allowed to air dry for 5 minutes prior to imaging on a Talos L120C electron microscope at 57K magnification (Gatan camera).

### Immunogenicity Study

An immunogenicity study was undertaken in naïve CD-1 mice to evaluate the immune responses to soluble fHBP antigen, fHBP fused to CompA, or VLPs displaying fHBP. A vehicle administered negative control group and a group immunized with a commercial MenB-FHbp Meningococcal Group B vaccine, Trumenba, was included in the study. Trumenba is a bivalent vaccine composed of equal amounts of peptide 45 and peptide 55 lipidated fHBP antigens. Mice were immunized on days 0 and 35 at the dosages and formulations indicated in Table 6. The soluble fHBP, CompA-fHBP and Tremenba were administered at equivalent antigen content. The fHPB VLPs were administered at the equivalent antigen content (1 μg) and 5-fold and 25-fold lower antigen content. Serum was collected on day 49 for serum bactericidal antibody assays with human complement (hSBA) and binding assays. hSBAs were performed against six MenB isolates: M15 240313, M08 240157, M17 240102, M17 240832, M18 240043, M01 240355. Isolates M15 240313 and M18 240043 express peptides 14 and 15, respectively, which have approximately 92% amino acid sequence identity with peptide 55. M17 240102 expresses peptide 13 which shares approximately 89% amino acid sequence identity with peptide 55. Peptides 1 and 4 are expressed by M08 240157 and M17 240832, respectively. Both share 87% amino acid sequence identity with peptide 55. Finally, M01 240355 expressed peptide 31 which shares roughly 68% amino acid sequence identity with peptide 55. Statistical analyses were performed using the nonparametric Mann-Whitney test using GraphPad Prism 10.1.0. Peptide 45 and peptide 55 binding assays were also performed using the Day 49 sera. Recombinant peptide 45 and peptide 55 were conjugated to MagPlex® Microspheres (Diasorin) using the xMAP® Antibody Coupling kit (Diasorin) following the manufacturer's instructions. The conguation of both peptides to the microspheres was confirmed using the mAb, JAR41. Conjugated microspheres were mixed and added to 96-well plates. Serially diluted serum and JAR41 were added to the microspheres, incubated at room temperature with 800 rpm shaking, then stored at 4° C. overnight. The next day, the 96-well plates were shaken for 30 minutes at room temperature and washed. A PE Goat anti-mouse IgG antibody was added to each well and the plates were incubated while shaking for one hour at room temperature. The plates were then washed and stored at 4° C. prior to reading on a Lumincx™ xMAP™ INTELLIFLEX (ThermoFisher). A set event collection minimum of 100 was used during data collection. Data were analyzed using GraphPad Prism. Titers were calculated as the EC50s from a 4PL curve fit of the serial dilutions. Curves were constrained at the top plateau and the background mean fluorescence intensity (MFI) for the given antigen. The lower limit was assigned as undiluted sera. Samples with an EC50 below that limit were assigned the value of 0.5.

To evaluate IgG1 and IgG2a levels, serum from each mouse was diluted 1:50,000. The peptide 55 binding assay was performed as described above using PE rat anti-mouse IgG1 and PE rat anti-mouse IgG2a (Biolegend) secondaries at a 1:100 dilution. MFI values were plotted in GraphPad Prism.

### Example 2

This example describes characterization and immunogenicity of fHBP-displaying protein nanostructure.

A fusion protein containing fHBP as an antigen was constructed with an I53-50A multimerization domain fused to the C-terminus of the fHBP antigen with a 12 residue Glycine/Serine linker. A His6 tag was included at the C-terminus of the I53-50A for purification. The fHBP soluble protein and CompA-fHBP protein expressed at high levels and were purified to >80% purity by IMAC (FIG. 2).

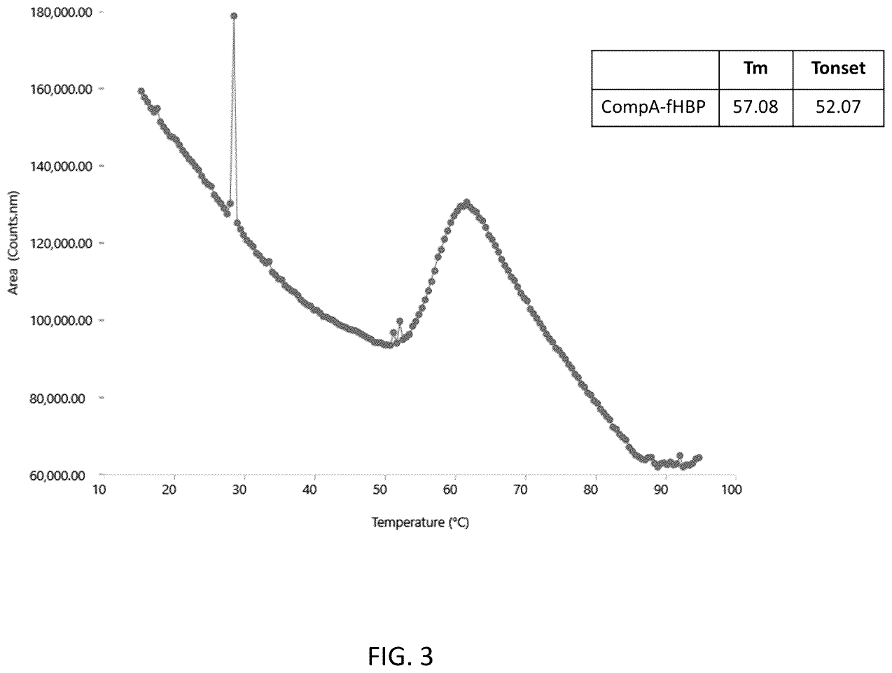

The Tonset and Tm for the CompA-fHBP were determined by nanoDSF (FIG. 3). A Tonset approximately 52° C. and a Tm of approximately 57° C. was observed.

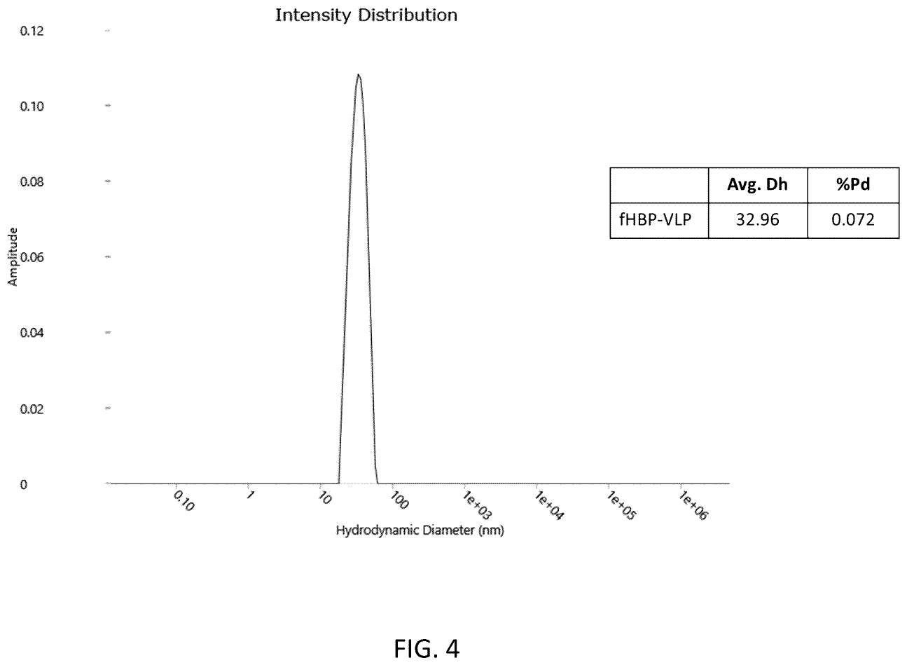

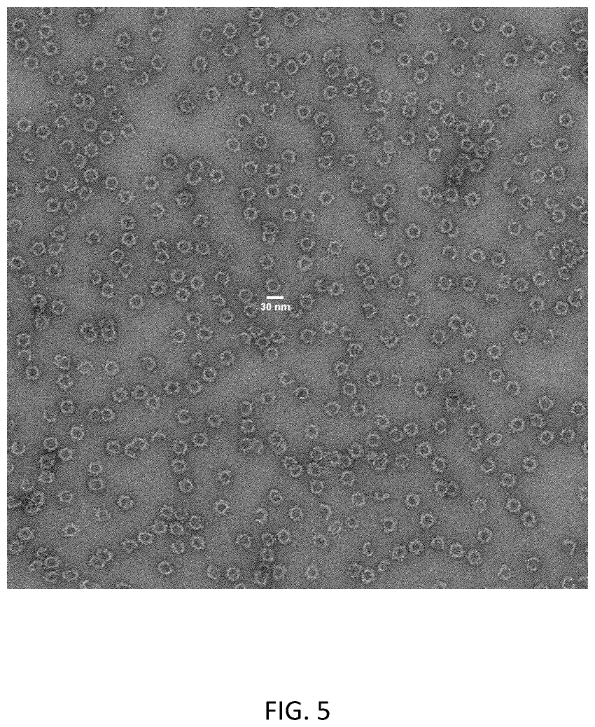

The CompA-fHBP was assembled with I53-50B to generate VLPs that were purified from unincorporated components by SEC. DLS demonstrated the VLPs were monodisperse and of the expected size (FIG. 4). The VLPs were imaged by nsEM and show monodisperse VLPs with the expected structure (FIG. 5) and VLPs run on a reduced SDS PAGE gel displayed both components (FIG. 2).

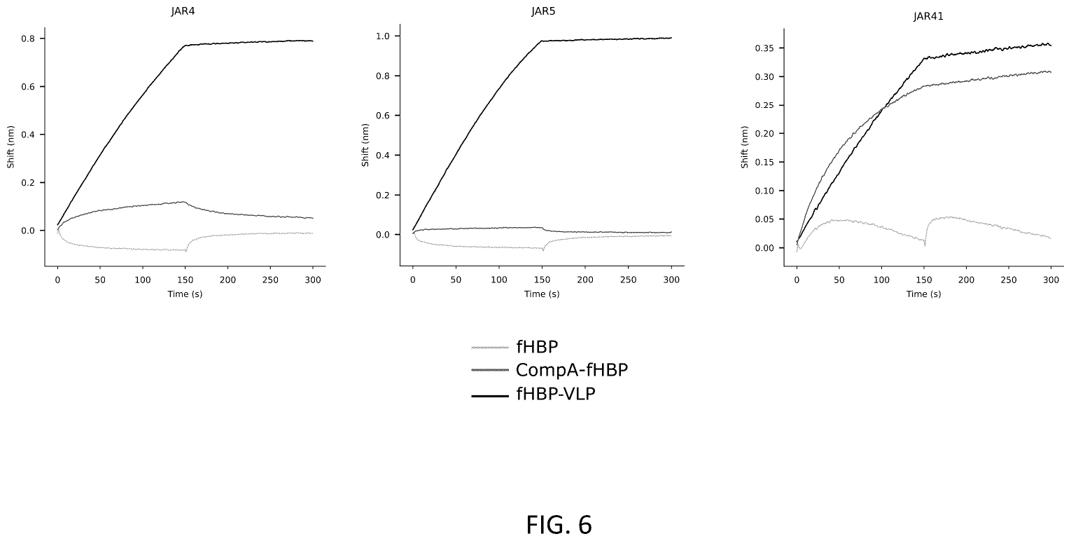

The fHBP soluble protein, CompA-fHBP, and fHBP VLPs were evaluated by BLI for binding to three commercial mAbs, JAR4, JAR5, and JAR41. JAR4 and JAR5 did not appreciably bind the soluble fHBP protein, minimal binding was observed to CompA-fHBP and significant binding was seen with the multivalent VLPs. Likely the low affinity binding of the Abs to the fHBP antigen were stabilized due to avidity with the multivalent VLPs. Weak binding of JAR41 was seen with the soluble protein and strong binding observed with the CompA-fHBP and fHBP VLPs (FIG. 6).

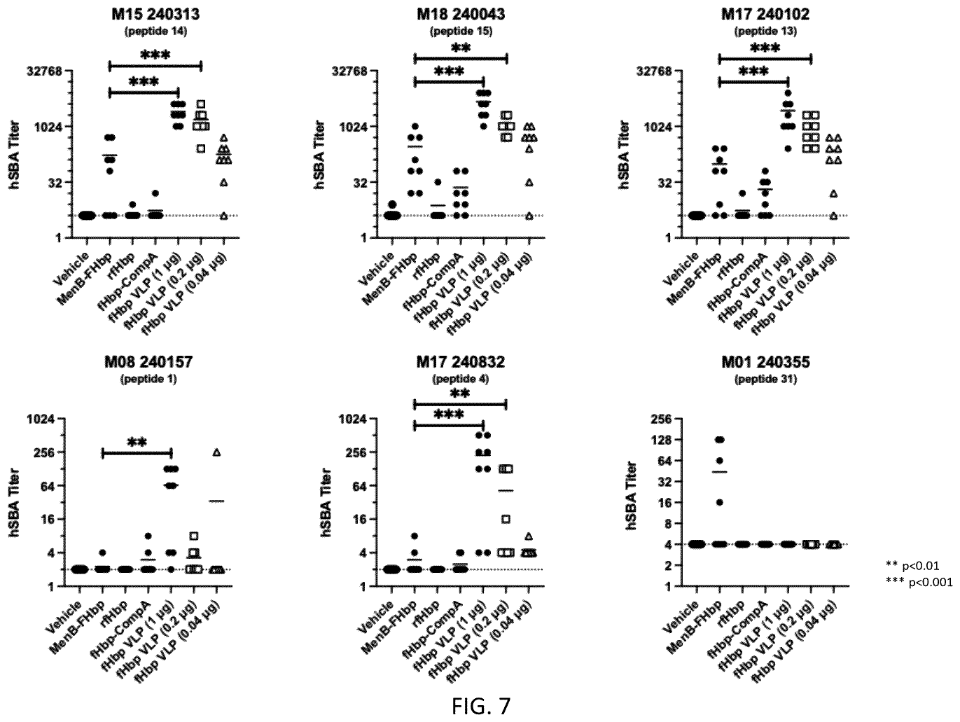

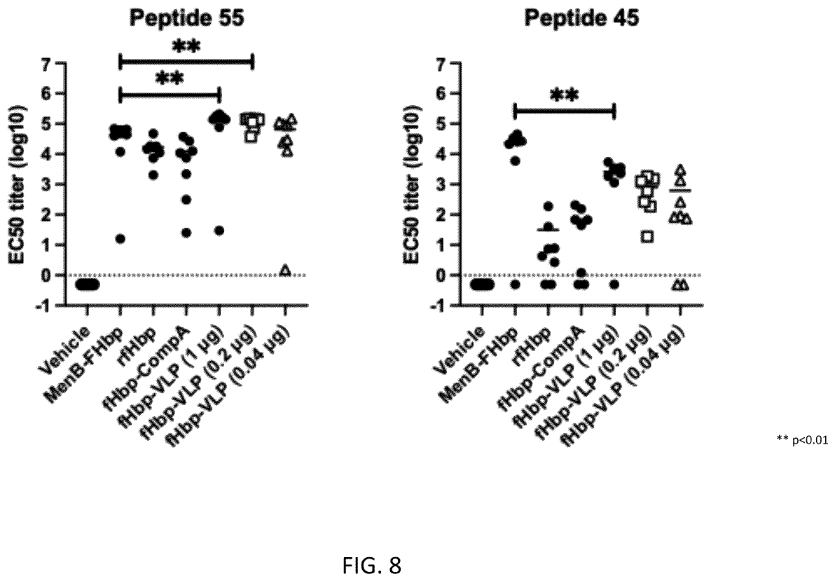

An immunogenicity study was performed to evaluate the hSBA titers induced by the fHBP antigen (peptide 55) when administered equivalent molar antigenic dosages as a soluble protein (fHBP), fused to CompA (CompA-fHBP), or displayed on VLPs (fHBP VLP). A commercial vaccine Trumenba was run as a control. All animals were immunized twice on days 0 and 35. The dosages and formulations for immunization is indicated in Table 6. Day 49 serum samples were evaluated in hSBA assays against six isolates: M15 240313, M08 240157, M17 240102, M17 240832, M18 240043, M01 240355. Isolates M15 240313 and M18 240043 express peptides 14 and 15, respectively, which have approximately 92% amino acid sequence identity with peptide 55. M17 240102 expresses peptide 13 which shares approximately 89% amino acid sequence identity with peptide 55. Peptides 1 and 4 are expressed by M08 240157 and M17 240832, respectively. Both share 87% amino acid sequence identity with peptide 55. Finally, M01 240355 expressed peptide 31 which shares roughly 68% amino acid sequence identity with peptide 55. hSBA titers induced by fHBP VLPs were significantly higher (Mann-Whitney test) than titers induced by Trumenba against all isolates except M01 240355 at equivalent antigen content (1 μg VLPs). Animals immunized with 5-fold lower (0.2 μg VLPs) antigen content also had significantly higher hSBA titers against isolates M15 240313, M18 240043, M17 240102, and M17 240832 compared to animals immunized with Trumenba (“MenB-FHbp”). The fHPB VLPs induced robust hSBA titers against isolates expressing two of the more distant fHBP peptides 1 and 4 while Trumenba did not. However, immunization with Trumenba did elicit hSBA titers against an isolate expressing peptide 31, which shares 97% amino acid sequence identify with the other fHBP component of the Trumenba vaccine, peptide 45 (FIG. 7). In addition to hSBA titers, binding titers against peptide 55 and peptide 45 were also determined using Day 49 sera. Peptide 55 binding titers induced by fHBP VLPs were significantly higher (Mann-Whitney test) than titers induced by Trumenba at equivalent antigen content (VLPs at 1 μg) and 5-fold lower (0.2 μg VLPs) antigen content. As expected, peptide 45 binding titers induced by Trumenba were significantly higher (Mann-Whitney test) than those induced by fHBP VLPs (FIG. 8).

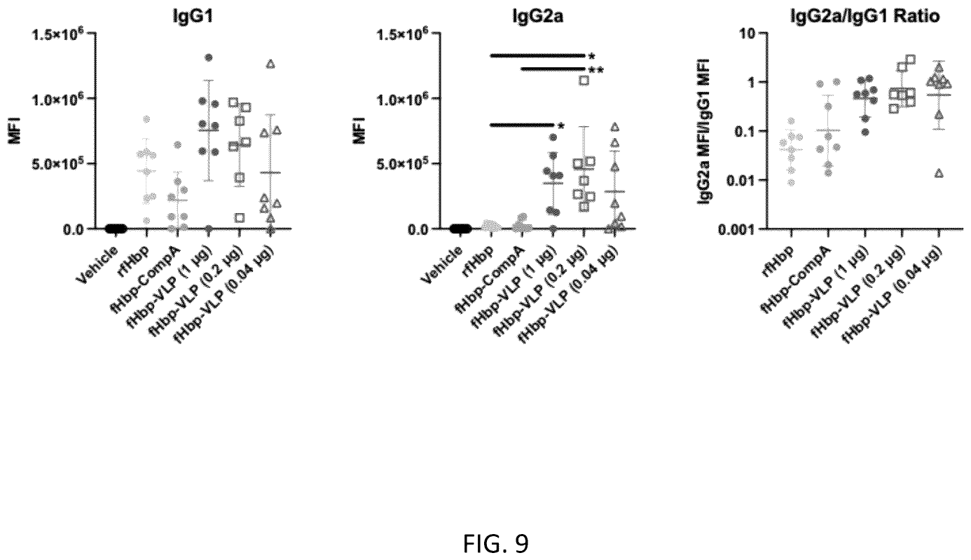

We also evaluated the anti-peptide 55 IgG1 and IgG2a levels in mice immunized with rfHbp, fHbp-CompA, and fHbp VLPs. There was a general trend towards the fHbp VLP groups having higher IgG1 levels than the rfHbp and fHbp-CompA groups. IgG2a levels were significantly higher in mice immunized with fHbp VLPs compared to mice immunized with rfHbp or fHbp-CompA. Therefore, mice immunized with rfHbp and fHbp-CompA had a bias towards IgG1. In contrast, mice immunized with fHbp VLPs had relatively comparable levels of IgG1 and IgG2a (FIG. 9).

TABLE 6

| | | Test | Protein dose per | | Immunization | Blood |
|---|---|---|---|---|---|---|
| Group | N | Article | Animal (ug) | Formulation | Schedule | Collection |
| 1 | 24 | Vehicle | N/A | Adda Vax | Day 0 and 35 | Day 49 |
| 2 | 24 | Trumenba | 0.8 | Adju-Phos | | |
| 3 | 24 | fHbp | 0.4 | Adda Vax | | |
| 4 | 24 | CompA- | 0.75 | | | |
| | | fHBP | | | | |
| 5 | 24 | fHbp VLP | 1 | | | |
| 6 | 24 | fHbp VLP | 0.2 | | | |
| 7 | 24 | fHbp VLP | 0.04 | | | |

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A protein nanostructure, comprising a first component, and optionally a second component,
wherein the first component comprises a factor H binding protein (fHBP), optionally of Neisseria meningitidis or Neisseria gonorrhoeae or any Neisseria bacterium, and a first polypeptide comprising a first assembly domain; and
wherein the second component comprises a second polypeptide comprising a second assembly domain,
wherein the first assembly domain and the second assembly domain each comprises a polypeptide sequence at least 50%, at least 60%, at least 70%, at least 75%, at least 80%, at least 85%, at least 90%, at least 95%, at least 96%, at least 97%, at least 98%, at least 99%, or 100% identical to a polypeptide sequence in Table 1.

2. The protein nanostructure of claim 1, wherein the fHBP comprises a polypeptide sequence at least 70%, at least 80%, at least 90%, at least 95%, at least 96%, at least 97%, at least 98%, at least 99%, or 100% identical to any polypeptide sequence in Table 2.

3. The protein nanostructure of claim 1, wherein the first component comprises a fusion protein comprising, in N- to C-terminal order, the fHBP, a polypeptide linker, and a I53-50A assembly domain.

4. The protein nanostructure of claim 3, wherein the polypeptide linker comprises between 6 and 18 amino acids and/or the polypeptide linker is a glycine-serine (Gly-Ser) linker and/or the polypeptide linker is any sequence in Table 3.

5.-12. (canceled)

13. The protein nanostructure of claim 3, wherein the fusion protein comprises a polypeptide sequence at least 85%, at least 90%, at least 95%, at least 96%, at least 97%, at least 98%, at least 99%, or 100% identical to any sequence in Table 4.

14. The protein nanostructure of claim 1, wherein the first component is a trimeric component comprising three copies of the first polypeptide and/or wherein the second component is a pentamer comprising five copies of the second polypeptide.

15. (canceled)

16. The protein nanostructure of claim 1, wherein the protein nanostructure comprises 20 copies of the first component and/or wherein the protein nanostructure further comprises 12 copies of the second component.

17. (canceled)

18. The protein nanostructure of claim 1, wherein the protein nanostructure is an icosahedral nanostructure.

19. The protein nanostructure of claim 3, wherein the I53-50A assembly domain comprises a polypeptide sequence at least 70%, at least 80%, at least 90%, at least 95%, at least 96%, at least 97%, at least 98%, at least 98%, at least 99%, or 100% identical to any one of SEQ ID NOs: 7, 29-31, 39, 53, 144, or 132.

20. The protein nanostructure of claim 1, wherein an I53-50B assembly domain comprises a polypeptide sequence at least 70%, at least 80%, at least 90%, at least 95%, at least 96%, at least 97%, at least 98%, at least 98%, at least 99%, or 100% identical to any one of SEQ ID NOs: 8, 32-34, or 40.

21. A polynucleotide encoding the components of the protein nanostructure of claim 1.

22.-23. (canceled)

24. The polynucleotide of claim 21, wherein the polynucleotide is any sequence in Table 5.

25. A pharmaceutical composition comprising the protein nanostructure of claim 1 and one or more pharmaceutically acceptable diluents, adjuvants, or excipients.

26.-28. (canceled)

29. A fusion protein according to claim 3.

30. A polynucleotide encoding the fusion protein of claim 29.

31. A host cell, comprising the polynucleotide of claim 30.

32.-33. (canceled)

34. A method of manufacturing a vaccine, comprising culturing the host cell of claim 31 in a culture medium so that the host cell secretes the first component into the culture medium; and optionally purifying the first component from the culture medium.

35. The method of claim 34, comprising mixing the first component with a second component, wherein the second component multimerizes with the first component to form a protein nanostructure; and optionally purifying the protein nanostructure.

36. (canceled)

37. A method of immunizing a subject against infection by meningococcal disease, the method comprising administering the protein nanostructure of claim 1.

38. A pharmaceutical composition comprising the polynucleotide of claim 21 and one or more pharmaceutically acceptable diluents, adjuvants, or excipients.

39. A method of immunizing a subject against infection by meningococcal disease, the method comprising administering the pharmaceutical composition of claim 38.
